(19) United States
(12) Patent Application Publication
(10) Pub. No.: US 2023/0192720 A1
(43) Pub. Date: Jun. 22, 2023

(54) MACROCYCLIC COMPOUNDS

(71) Applicant: ZENO MANAGEMENT, INC., San Diego, CA (US)

(72) Inventors: Junhu Zhang, San Diego, CA (US); Peter Qinhua Huang, San Diego, CA (US); Kevin Duane Bunker, Escondido, CA (US); Sobhana Babu Boga, San Diego, CA (US); Sunny Abraham, San Diego, CA (US); Brant Clayton Boren, San Diego, CA (US); Wanlong Jiang, San Diego, CA (US); Sunil Paliwal, Monroe Township, NJ (US)

(21) Appl. No.: 17/757,437

(22) PCT Filed: Aug. 6, 2020
(86) PCT No.: PCT/US2020/045255

§ 371 Date: Jun. 15, 2022

Related U.S. Application Data
(60) Provisional application No. 62/949,784, filed on Dec. 18, 2019.
(60) Provisional application No. 63/032,342, filed on May 29, 2020.

(51) Int. Cl. C07D 515/22

(52) U.S. Cl. CPC C07D 515/22

(57) ABSTRACT

The present application discloses compounds of Formula (I). Such compounds, pharmaceutically acceptable salts and compositions thereof, are inhibitors of Mcl-1 proteins and are useful in treating diseases and conditions characterized by excessive cellular proliferation such as cancer.

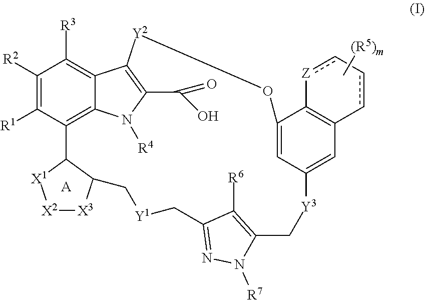

### INCORPORATION BY REFERENCE TO ANY PRIORITY APPLICATIONS

Any and all applications for which a foreign or domestic priority claim is identified, for example, in the Application Data Sheet or Request as filed with the present application, are hereby incorporated by reference under 37 CFR 1.57, and Rules 4.18 and 20.6, including U.S. Provisional Application Nos. 62/949,784, filed Dec. 18, 2019, and 63/032,342, filed May 29, 2020.

### FIELD

The present application relates to compounds that are Mcl-1 inhibitors and methods of using them to treat conditions characterized by excessive cellular proliferation, such as cancer.

### DESCRIPTION

Mcl-1 (myeloid cell leukemia-1) is a member of the Bcl-2 family of proteins. MCL-1 is widely expressed in human tissues and is primarily located in the mitochondria in cells. Upregulation of Mcl-1 occurs in different cancer types. Additionally, overexpression of Mcl-1 has been linked to drug resistance to several cancer therapies.

### SUMMARY

Some embodiments provide a compound of Formula (I), or a pharmaceutically acceptable salt thereof.

Some embodiments disclosed herein relate to a pharmaceutical composition that can include an effective amount of one or more of compounds of Formula (I), or a pharmaceutically acceptable salt thereof, and a pharmaceutically acceptable carrier, diluent, excipient or combination thereof.

Some embodiments described herein relate to a method for ameliorating and/or treating a cancer described herein that can include administering an effective amount of a compound described herein (for example, a compound of Formula (I), or a pharmaceutically acceptable salt thereof) or a pharmaceutical composition that includes an effective amount of a compound described herein (for example, a compound of Formula (I), or a pharmaceutically acceptable salt thereof) to a subject having a cancer described herein. Other embodiments described herein relate to the use of an effective amount of a compound described herein (for example, a compound of Formula (I), or a pharmaceutically acceptable salt thereof) or a pharmaceutical composition that includes an effective amount of a compound described herein (for example, a compound of Formula (I), or a pharmaceutically acceptable salt thereof) in the manufacture of a medicament for ameliorating and/or treating a cancer described herein. Still other embodiments described herein relate to an effective amount of a compound described herein (for example, a compound of Formula (I), or a pharmaceutically acceptable salt thereof) or a pharmaceutical composition that includes an effective amount of a compound described herein (for example, a compound of Formula (I), or a pharmaceutically acceptable salt thereof) for ameliorating and/or treating a cancer described herein.

Some embodiments described herein relate to a method for inhibiting replication of a malignant growth or a tumor that can include contacting the growth or the tumor with an effective amount of a compound described herein (for example, a compound of Formula (I), or a pharmaceutically acceptable salt thereof) or a pharmaceutical composition that includes an effective amount of a compound described herein (for example, a compound of Formula (I), or a pharmaceutically acceptable salt thereof), wherein the malignant growth or tumor is due to a cancer described herein. Other embodiments described herein relate to the use of an effective amount of a compound described herein (for example, a compound of Formula (I), or a pharmaceutically acceptable salt thereof) or a pharmaceutical composition that includes an effective amount of a compound described herein (for example, a compound of Formula (I), or a pharmaceutically acceptable salt thereof) in the manufacture of a medicament for inhibiting replication of a malignant growth or a tumor, wherein the malignant growth or tumor is due to a cancer described herein. Still other embodiments described herein relate to an effective amount of a compound described herein (for example, a compound of Formula (I), or a pharmaceutically acceptable salt thereof) or a pharmaceutical composition that includes an effective amount of a compound described herein (for example, a compound of Formula (I), or a pharmaceutically acceptable salt thereof) for inhibiting replication of a malignant growth or a tumor, wherein the malignant growth or tumor is due to a cancer described herein.

Some embodiments described herein relate to a method for ameliorating or treating a cancer described herein that can include contacting a malignant growth or a tumor with an effective amount of a compound described herein (for example, a compound of Formula (I), or a pharmaceutically acceptable salt thereof) or a pharmaceutical composition that includes an effective amount of a compound described herein (for example, a compound of Formula (I), or a pharmaceutically acceptable salt thereof) to a subject having a cancer described herein. Other embodiments described herein relate to the use of an effective amount of a compound described herein (for example, a compound of Formula (I), or a pharmaceutically acceptable salt thereof) or a pharmaceutical composition that includes an effective amount of a compound described herein (for example, a compound of Formula (I), or a pharmaceutically acceptable salt thereof) in the manufacture of a medicament for ameliorating or treating a cancer described herein that can include contacting a malignant growth or a tumor, wherein the malignant growth or tumor is due to a cancer described herein. Still other embodiments described herein relate to an effective amount of a compound described herein (for example, a compound of Formula (I), or a pharmaceutically acceptable salt thereof) or a pharmaceutical composition that includes an effective amount of a compound described herein (for example, a compound of Formula (I), or a pharmaceutically acceptable salt thereof) for ameliorating or treating a cancer described herein that can include contacting a malignant growth or a tumor, wherein the malignant growth or tumor is due to a cancer described herein.

Some embodiments described herein relate to a method for inhibiting the activity of Mcl-1 in a cell that can include providing an effective amount of a compound described herein (for example, a compound of Formula (I), or a pharmaceutically acceptable salt thereof) or a pharmaceutical composition that includes an effective amount of a compound described herein (for example, a compound of Formula (I), or a pharmaceutically acceptable salt thereof) to a cancer cell from a cancer described herein. Other embodiments described herein relate to the use of an effective amount of a compound described herein (for example, a compound of Formula (I), or a pharmaceutically acceptable salt thereof) or a pharmaceutical composition that includes an effective amount of a compound described herein (for example, a compound of Formula (I), or a pharmaceutically acceptable salt thereof) in the manufacture of a medicament for inhibiting the activity of Mcl-1. Still other embodiments described herein relate to an effective amount of a compound described herein (for example, a compound of Formula (I), or a pharmaceutically acceptable salt thereof) or a pharmaceutical composition that includes an effective amount of a compound described herein (for example, a compound of Formula (I), or a pharmaceutically acceptable salt thereof) for inhibiting the activity of Mcl-1.

Some embodiments described herein relate to a method for ameliorating or treating a cancer described herein that can include inhibiting the activity of Mcl-1 using an effective amount of a compound described herein (for example, a compound of Formula (I), or a pharmaceutically acceptable salt thereof) or a pharmaceutical composition that includes an effective amount of a compound described herein (for example, a compound of Formula (I), or a pharmaceutically acceptable salt thereof). Other embodiments described herein relate to the use of an effective amount of a compound described herein (for example, a compound of Formula (I), or a pharmaceutically acceptable salt thereof) or a pharmaceutical composition that includes an effective amount of a compound described herein (for example, a compound of Formula (I), or a pharmaceutically acceptable salt thereof) in the manufacture of a medicament for ameliorating or treating a cancer described herein by inhibiting the activity of Mcl-1. Still other embodiments described herein relate to an effective amount of a compound described herein (for example, a compound of Formula (I), or a pharmaceutically acceptable salt thereof) or a pharmaceutical composition that includes an effective amount of a compound described herein (for example, a compound of Formula (I), or a pharmaceutically acceptable salt thereof) for ameliorating or treating a cancer described herein by inhibiting the activity of Mcl-1.

### DETAILED DESCRIPTION

Myeloid Cell Leukemia 1 (Mcl-1) is an important anti-apoptotic member of the BCL-2 family of proteins and a master regulator of cell survival. Amplification of the MCL1 gene and/or overexpression of the Mcl-1 protein has been observed in multiple cancer types and is commonly implicated in tumor development. MCL1 is one of the most frequently amplified genes in human cancers. In many malignancies, Mcl-1 is a critical survival factor and it has been shown to mediate drug resistance to a variety of anti-cancer agents. Mcl-1 promotes cell survival by binding to pro-apoptotic proteins like Bim, Noxa, Bak, and Bax and neutralizing their death-inducing activities. Inhibition of Mcl-1 thereby releases these pro-apoptotic proteins, often leading to the induction of apoptosis in tumor cells dependent on Mcl-1 for survival. Therapeutically targeting Mcl-1 alone or in combination with other therapies, therefore, is a promising strategy to treat a multitude of malignancies and to overcome drug resistance in several human cancers.

### Definitions

Unless defined otherwise, all technical and scientific terms used herein have the same meaning as is commonly understood by one of ordinary skill in the art. All patents, applications, published applications and other publications referenced herein are incorporated by reference in their entirety unless stated otherwise. In the event that there are a plurality of definitions for a term herein, those in this section prevail unless stated otherwise.

Whenever a group is described as being “optionally substituted” that group may be unsubstituted or substituted with one or more of the indicated substituents. Likewise, when a group is described as being “unsubstituted or substituted” if substituted, the substituent(s) may be selected from one or more the indicated substituents. If no substituents are indicated, it is meant that the indicated “optionally substituted” or “substituted” group may be substituted with one or more group(s) individually and independently selected from alkyl, alkenyl, alkynyl, cycloalkyl, cycloalkenyl, aryl, heteroaryl, heterocyclyl, aryl(alkyl), cycloalkyl(alkyl), heteroaryl(alkyl), heterocyclyl(alkyl), hydroxy, alkoxy, acyl, cyano, halogen, thiocarbonyl, O-carbamyl, N-carbamyl, O-thiocarbamyl, N-thiocarbamyl, C-amido, N-amido, S-sulfonamido, N-sulfonamido, C-carboxy, O-carboxy, nitro, sulfenyl, sulfinyl, sulfonyl, haloalkyl, hydroxyalkyl, haloalkoxy, an amino, a mono-substituted amine group, a di-substituted amine group and an amine(C₁-C₆ alkyl).

As used herein, “Cₐ to C_{b}” in which “a” and “b” are integers refer to the number of carbon atoms in a group. The indicated group can contain from “a” to “b”, inclusive, carbon atoms. Thus, for example, a “C₁ to C₄ alkyl” group refers to all alkyl groups having from 1 to 4 carbons, that is, CH₃—, CH₃CH₂—, CH₃CH₂CH₂—, (CH₃)₂CH—, CH₃CH₂CH₂CH₂—, CH₃CH₂CH(CH₃)— and (CH₃)₃C—. If no “a” and “b” are designated, the broadest range described in these definitions is to be assumed.

If two “R” groups are described as being “taken together” the R groups and the atoms they are attached to can form a cycloalkyl, cycloalkenyl, aryl, heteroaryl or heterocycle. For example, without limitation, if R^{a} and R^{b} of an NR^{a}R^{b} group are indicated to be “taken together,” it means that they are covalently bonded to one another to form a ring:

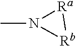

As used herein, the term “alkyl” refers to a fully saturated aliphatic hydrocarbon group. The alkyl moiety may be branched or straight chain. Examples of branched alkyl groups include, but are not limited to, iso-propyl, sec-butyl, t-butyl and the like. Examples of straight chain alkyl groups include, but are not limited to, methyl, ethyl, n-propyl, n-butyl, n-pentyl, n-hexyl, n-heptyl and the like. The alkyl group may have 1 to 30 carbon atoms (whenever it appears herein, a numerical range such as “1 to 30” refers to each integer in the given range; e.g., “1 to 30 carbon atoms” means that the alkyl group may consist of 1 carbon atom, 2 carbon atoms, 3 carbon atoms, etc., up to and including 30 carbon atoms, although the present definition also covers the occurrence of the term “alkyl” where no numerical range is designated). The alkyl group may also be a medium size alkyl having 1 to 12 carbon atoms. The alkyl group could also be a lower alkyl having 1 to 6 carbon atoms. An alkyl group may be substituted or unsubstituted.

The term “alkenyl” used herein refers to a monovalent straight or branched chain radical of from two to twenty carbon atoms containing a carbon double bond(s) including, but not limited to, 1-propenyl, 2-propenyl, 2-methyl-1-propenyl, 1-butenyl, 2-butenyl and the like. An alkenyl group may be unsubstituted or substituted.

The term “alkynyl” used herein refers to a monovalent straight or branched chain radical of from two to twenty carbon atoms containing a carbon triple bond(s) including, but not limited to, 1-propynyl, 1-butynyl, 2-butynyl and the like. An alkynyl group may be unsubstituted or substituted.

As used herein, “cycloalkyl” refers to a completely saturated (no double or triple bonds) mono- or multi-cyclic hydrocarbon ring system. When composed of two or more rings, the rings may be joined together in a fused, bridged or spiro fashion. As used herein, the term “fused” refers to two rings which have two atoms and one bond in common. As used herein, the term “bridged cycloalkyl” refers to compounds wherein the cycloalkyl contains a linkage of one or more atoms connecting non-adjacent atoms. As used herein, the term “spiro” refers to two rings which have one atom in common and the two rings are not linked by a bridge. Cycloalkyl groups can contain 3 to 30 atoms in the ring(s), 3 to 20 atoms in the ring(s), 3 to 10 atoms in the ring(s), 3 to 8 atoms in the ring(s) or 3 to 6 atoms in the ring(s). A cycloalkyl group may be unsubstituted or substituted. Examples of mono-cycloalkyl groups include, but are in no way limited to, cyclopropyl, cyclobutyl, cyclopentyl, cyclohexyl, cycloheptyl and cyclooctyl. Examples of fused cycloalkyl groups are decahydronaphthalenyl, dodecahydro-1H-phenalenyl and tetradecahydroanthracenyl; examples of bridged cycloalkyl groups are bicyclo[1.1.1]pentyl, adamantanyl and norbornanyl; and examples of spiro cycloalkyl groups include spiro[3.3]heptane and spiro[4.5]decane.

As used herein, “cycloalkenyl” refers to a mono- or multi-cyclic hydrocarbon ring system that contains one or more double bonds in at least one ring; although, if there is more than one, the double bonds cannot form a fully delocalized pi-electron system throughout all the rings (otherwise the group would be “aryl,” as defined herein). Cycloalkenyl groups can contain 3 to 10 atoms in the ring(s), 3 to 8 atoms in the ring(s) or 3 to 6 atoms in the ring(s). When composed of two or more rings, the rings may be connected together in a fused, bridged or spiro fashion. A cycloalkenyl group may be unsubstituted or substituted.

As used herein, “aryl” refers to a carbocyclic (all carbon) monocyclic or multicyclic aromatic ring system (including fused ring systems where two carbocyclic rings share a chemical bond) that has a fully delocalized pi-electron system throughout all the rings. The number of carbon atoms in an aryl group can vary. For example, the aryl group can be a C₆-C₁₄ aryl group, a C₆-C₁₀ aryl group or a C₆ aryl group. Examples of aryl groups include, but are not limited to, benzene, naphthalene and azulene. An aryl group may be substituted or unsubstituted.

As used herein, “heteroaryl” refers to a monocyclic or multicyclic aromatic ring system (a ring system with fully delocalized pi-electron system) that contain(s) one or more heteroatoms (for example, 1, 2 or 3 heteroatoms), that is, an element other than carbon, including but not limited to, nitrogen, oxygen and sulfur. The number of atoms in the ring(s) of a heteroaryl group can vary. For example, the heteroaryl group can contain 4 to 14 atoms in the ring(s), 5 to 10 atoms in the ring(s) or 5 to 6 atoms in the ring(s), such as nine carbon atoms and one heteroatom; eight carbon atoms and two heteroatoms; seven carbon atoms and three heteroatoms; eight carbon atoms and one heteroatom; seven carbon atoms and two heteroatoms; six carbon atoms and three heteroatoms; five carbon atoms and four heteroatoms; five carbon atoms and one heteroatom; four carbon atoms and two heteroatoms; three carbon atoms and three heteroatoms; four carbon atoms and one heteroatom; three carbon atoms and two heteroatoms; or two carbon atoms and three heteroatoms. Furthermore, the term “heteroaryl” includes fused ring systems where two rings, such as at least one aryl ring and at least one heteroaryl ring or at least two heteroaryl rings, share at least one chemical bond. Examples of heteroaryl rings include, but are not limited to, furan, furazan, thiophene, benzothiophene, phthalazine, pyrrole, oxazole, benzoxazole, 1,2,3-oxadiazole, 1,2,4-oxadiazole, thiazole, 1,2,3-thiadiazole, 1,2,4-thiadiazole, benzothiazole, imidazole, benzimidazole, indole, indazole, pyrazole, benzopyrazole, isoxazole, benzoisoxazole, isothiazole, triazole, benzotriazole, thiadiazole, tetrazole, pyridine, pyridazine, pyrimidine, pyrazine, purine, pteridine, quinoline, isoquinoline, quinazoline, quinoxaline, cinnoline and triazine. A heteroaryl group may be substituted or unsubstituted.

As used herein, “heterocyclyl” refers to three-, four-, five-, six-, seven-, eight-, nine-, ten-, up to 18-membered monocyclic, bicyclic and tricyclic ring system wherein carbon atoms together with from 1 to 5 heteroatoms constitute said ring system. A heterocycle may optionally contain one or more unsaturated bonds situated in such a way, however, that a fully delocalized pi-electron system does not occur throughout all the rings. The heteroatom(s) is an element other than carbon including, but not limited to, oxygen, sulfur and nitrogen. A heterocycle may further contain one or more carbonyl or thiocarbonyl functionalities, so as to make the definition include oxo-systems and thio-systems such as lactams, lactones, cyclic imides, cyclic thioimides and cyclic carbamates. When composed of two or more rings, the rings may be joined together in a fused, bridged or spiro fashion. As used herein, the term “fused” refers to two rings which have two atoms and one bond in common. As used herein, the term “bridged heterocyclyl” refers to compounds wherein the heterocyclyl contains a linkage of one or more atoms connecting non-adjacent atoms. As used herein, the term “spiro” refers to two rings which have one atom in common and the two rings are not linked by a bridge. Heterocyclyl group can contain 3 to 30 atoms in the ring(s), 3 to 20 atoms in the ring(s), 3 to 10 atoms in the ring(s), 3 to 8 atoms in the ring(s) or 3 to 6 atoms in the ring(s). For example, five carbon atoms and one heteroatom; four carbon atoms and two heteroatoms; three carbon atoms and three heteroatoms; four carbon atoms and one heteroatom; three carbon atoms and two heteroatoms; two carbon atoms and three heteroatoms; one carbon atom and four heteroatoms; three carbon atoms and one heteroatom; or two carbon atoms and one heteroatom. Additionally, any nitrogens in a heterocyclyl may be quaternized. Heterocyclyl groups may be unsubstituted or substituted. Examples of such “heterocyclyl” groups include but are not limited to, 1,3-dioxin, 1,3-dioxane, 1,4-dioxane, 1,2-dioxolane, 1,3-dioxolane, 1,4-dioxolane, 1,3-oxathiane, 1,4-oxathiin, 1,3-oxathiolane, 1,3-dithiole, 1,3-dithiolane, 1,4-oxathiane, tetrahydro-1,4-thiazine, 2H-1,2-oxazine, maleimide, succinimide, barbituric acid, thiobarbituric acid, dioxopiperazine, hydantoin, dihydrouracil, trioxane, hexahydro-1,3,5-triazine, imidazoline, imidazolidine, isoxazoline, isoxazolidine, oxazoline, oxazolidine, oxazolidinone, thiazoline, thiazolidine, morpholine, oxirane, piperidine N-Oxide, piperidine, piperazine, pyrrolidine, azepane, pyrrolidone, pyrrolidione, 4-piperidone, pyrazoline, pyrazolidine, 2-oxopyrrolidine, tetrahydropyran, 4H-pyran, tetrahydrothiopyran, thiamorpholine, thiamorpholine sulfoxide, thiamorpholine sulfone and their benzo-fused analogs (e.g., benzimidazolidinone, tetrahydroquinoline and/or 3,4-methylenedioxyphenyl). Examples of spiro heterocyclyl groups include 2-azaspiro[3.3]heptane, 2-oxaspiro[3.3]heptane, 2-oxa-6-azaspiro[3.3]heptane, 2,6-diazaspiro[3.3]heptane, 2-oxaspiro[3.4]octane and 2-azaspiro[3.4]octane.

As used herein, “cycloalkyl(alkyl)” refer to an cycloalkyl group connected, as a substituent, via a lower alkylene group. The lower alkylene and cycloalkyl group of an cycloalkyl(alkyl) may be substituted or unsubstituted. Examples include but are not limited to cyclopropyl(alkyl), cyclobutyl(alkyl), cyclopentyl(alkyl) and cyclohexyl(alkyl).

As used herein, “aryl(alkyl)” refer to an aryl group connected, as a substituent, via a lower alkylene group. The lower alkylene and aryl group of an aryl(alkyl) may be substituted or unsubstituted. Examples include but are not limited to benzyl, 2-phenylalkyl, 3-phenylalkyl and naphthylalkyl.

As used herein, “heteroaryl(alkyl)” refer to a heteroaryl group connected, as a substituent, via a lower alkylene group. The lower alkylene and heteroaryl group of heteroaryl(alkyl) may be substituted or unsubstituted. Examples include but are not limited to 2-thienylalkyl, 3-thienylalkyl, furylalkyl, thienylalkyl, pyrrolylalkyl, pyridylalkyl, isoxazolylalkyl and imidazolylalkyl and their benzo-fused analogs.

A “heterocyclyl(alkyl)” refer to a heterocyclic group connected, as a substituent, via a lower alkylene group. The lower alkylene and heterocyclyl of a heterocyclyl(alkyl) may be substituted or unsubstituted. Examples include but are not limited tetrahydro-2H-pyran-4-yl(methyl), piperidin-4-yl(ethyl), piperidin-4-yl(propyl), tetrahydro-2H-thiopyran-4-yl(methyl) and 1,3-thiazinan-4-yl(methyl).

As used herein, “lower alkylene groups” are straight-chained —CH₂— tethering groups, forming bonds to connect molecular fragments via their terminal carbon atoms. Examples include but are not limited to methylene (—CH₂—), ethylene (—CH₂CH₂—), propylene (—CH₂CH₂CH₂—) and butylene (—CH₂CH₂CH₂CH₂—). A lower alkylene group can be substituted by replacing one or more hydrogen of the lower alkylene group and/or by substituting both hydrogens on the same carbon with a cycloalkyl group

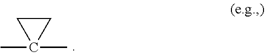

As used herein, the term “hydroxy” refers to a —OH group.

As used herein, “alkoxy” refers to the Formula —OR wherein R is an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl) is defined herein. A non-limiting list of alkoxys are methoxy, ethoxy, n-propoxy, 1-methylethoxy (iso-propoxy), n-butoxy, iso-butoxy, sec-butoxy, tert-butoxy, phenoxy and benzoxy. An alkoxy may be substituted or unsubstituted.

As used herein, “acyl” refers to a hydrogen, alkyl, alkenyl, alkynyl, aryl, heteroaryl, heterocyclyl, aryl(alkyl), heteroaryl(alkyl) and heterocyclyl(alkyl) connected, as substituents, via a carbonyl group. Examples include formyl, acetyl, propanoyl, benzoyl and acryl. An acyl may be substituted or unsubstituted.

A “cyano” group refers to a “—CN” group.

The term “halogen atom” or “halogen” as used herein, means any one of the radio-stable atoms of column 7 of the Periodic Table of the Elements, such as, fluorine, chlorine, bromine and iodine.

A “thiocarbonyl” group refers to a “—C(═S)R” group in which R can be the same as defined with respect to O-carboxy. A thiocarbonyl may be substituted or unsubstituted.

An “O-carbamyl” group refers to a “—OC(═O)N(R_{A}R_{B})” group in which R_{A} and R_{B} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An O-carbamyl may be substituted or unsubstituted.

An “N-carbamyl” group refers to an “ROC(═O)N(R_{A})—” group in which R and RA can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An N-carbamyl may be substituted or unsubstituted.

An “O-thiocarbamyl” group refers to a “—OC(═S)—N(R_{A}R_{B})” group in which R_{A} and R_{B} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An O-thiocarbamyl may be substituted or unsubstituted.

An “N-thiocarbamyl” group refers to an “ROC(═S)N(R_{A})—” group in which R and R_{A} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An N-thiocarbamyl may be substituted or unsubstituted.

A “C-amido” group refers to a “—C(═O)N(R_{A}R_{B})” group in which R_{A} and R_{B} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). A C-amido may be substituted or unsubstituted.

An “N-amido” group refers to a “RC(═O)N(R_{A})—” group in which R and R_{A} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An N-amido may be substituted or unsubstituted.

An “S-sulfonamido” group refers to a “—SO₂N(R_{A}R_{B})” group in which R_{A} and R_{B} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An S-sulfonamido may be substituted or unsubstituted.

An “N-sulfonamido” group refers to a “RSO₂N(R_{A})—” group in which R and R_{A} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An N-sulfonamido may be substituted or unsubstituted.

An “O-carboxy” group refers to a “RC(═O)O—” group in which R can be hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl), as defined herein. An O-carboxy may be substituted or unsubstituted.

The term “C-carboxy” refer to a “—C(═O)OR” group in which R can be the same as defined with respect to O-carboxy. A C-carboxy may be substituted or unsubstituted.

A “nitro” group refers to an “—NO₂” group.

A “sulfenyl” group refers to an “—SR” group in which R can be hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). A sulfenyl may be substituted or unsubstituted.

A “sulfinyl” group refers to an “—S(═O)—R” group in which R can be the same as defined with respect to sulfenyl. A sulfinyl may be substituted or unsubstituted.

A “sulfonyl” group refers to an “SO₂R” group in which R can be the same as defined with respect to sulfenyl. A sulfonyl may be substituted or unsubstituted.

As used herein, “haloalkyl” refers to an alkyl group in which one or more of the hydrogen atoms are replaced by a halogen (e.g., mono-haloalkyl, di-haloalkyl, tri-haloalkyl and polyhaloalkyl). Such groups include but are not limited to, chloromethyl, fluoromethyl, difluoromethyl, trifluoromethyl, 1-chloro-2-fluoromethyl, 2-fluoroisobutyl and pentafluoroethyl. A haloalkyl may be substituted or unsubstituted.

As used herein, “haloalkoxy” refers to an alkoxy group in which one or more of the hydrogen atoms are replaced by a halogen (e.g., mono-haloalkoxy, di-haloalkoxy and tri-haloalkoxy). Such groups include but are not limited to, chloromethoxy, fluoromethoxy, difluoromethoxy, trifluoromethoxy, 1-chloro-2-fluoromethoxy and 2-fluoroisobutoxy. A haloalkoxy may be substituted or unsubstituted.

The term “amino” as used herein refers to a —NH₂ group.

A “mono-substituted amine” group refers to a “—NHR_{A}” group in which R_{A} can be an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl), as defined herein. The RA may be substituted or unsubstituted. Examples of mono-substituted amino groups include, but are not limited to, —NH(methyl), —NH(phenyl) and the like.

A “di-substituted amine” group refers to a “—NR_{A}R_{B}” group in which R_{A} and R_{B} can be independently an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl), as defined herein. R_{A} and R_{B} can independently be substituted or unsubstituted. Examples of di-substituted amino groups include, but are not limited to, —N(methyl)₂, —N(phenyl)(methyl), —N(ethyl)(methyl) and the like.

As used herein, “amine(alkyl)” group refers to an -(alkylene)-NR′R″ radical where R′ and R″ are independently hydrogen or alkyl as defined herein. An amine(alkyl) may be substituted or unsubstituted. Examples of amine(alkyl) groups include, but are not limited to, —CH₂NH(methyl), —CH₂NH(phenyl), —CH₂CH₂NH(methyl), —CH₂CH₂NH(phenyl), —CH₂N(methyl)₂, —CH₂N(phenyl)(methyl), —NCH₂(ethyl)(methyl), —CH₂CH₂N(methyl)₂, —CH₂CH₂N(phenyl)(methyl), —NCH₂CH₂(ethyl)(methyl) and the like.

Where the number of substituents is not specified (e.g. haloalkyl), there may be one or more substituents present. For example, “haloalkyl” may include one or more of the same or different halogens. As another example, “C₁-C₃ alkoxyphenyl” may include one or more of the same or different alkoxy groups containing one, two or three atoms.

As used herein, a radical indicates species with a single, unpaired electron such that the species containing the radical can be covalently bonded to another species. Hence, in this context, a radical is not necessarily a free radical. Rather, a radical indicates a specific portion of a larger molecule. The term “radical” can be used interchangeably with the term “group.”

The term “pharmaceutically acceptable salt” refers to a salt of a compound that does not cause significant irritation to an organism to which it is administered and does not abrogate the biological activity and properties of the compound. In some embodiments, the salt is an acid addition salt of the compound. Pharmaceutical salts can be obtained by reacting a compound with inorganic acids such as hydrohalic acid (e.g., hydrochloric acid or hydrobromic acid), a sulfuric acid, a nitric acid and a phosphoric acid (such as 2,3-dihydroxypropyl dihydrogen phosphate). Pharmaceutical salts can also be obtained by reacting a compound with an organic acid such as aliphatic or aromatic carboxylic or sulfonic acids, for example formic, acetic, succinic, lactic, malic, tartaric, citric, ascorbic, nicotinic, methanesulfonic, ethanesulfonic, p-toluenesulfonic, trifluoroacetic, benzoic, salicylic, 2-oxopentanedioic or naphthalenesulfonic acid. Pharmaceutical salts can also be obtained by reacting a compound with a base to form a salt such as an ammonium salt, an alkali metal salt, such as a sodium, a potassium or a lithium salt, an alkaline earth metal salt, such as a calcium or a magnesium salt, a salt of a carbonate, a salt of a bicarbonate, a salt of organic bases such as dicyclohexylamine, N-methyl-D-glucamine, tris(hydroxymethyl)methylamine, C₁-C₇ alkylamine, cyclohexylamine, triethanolamine, ethylenediamine and salts with amino acids such as arginine and lysine. For compounds of Formula (I), those skilled in the art understand that when a salt is formed by protonation of a nitrogen-based group (for example, NH₂), the nitrogen-based group can be associated with a positive charge (for example, NH₂ can become NH₃⁺) and the positive charge can be balanced by a negatively charged counterion (such as Cl⁻).

It is understood that, in any compound described herein having one or more chiral centers, if an absolute stereochemistry is not expressly indicated, then each center may independently be of R-configuration or S-configuration or a mixture thereof. Thus, the compounds provided herein may be enantiomerically pure, enantiomerically enriched, racemic mixture, diastereomerically pure, diastereomerically enriched or a stereoisomeric mixture. In addition, it is understood that, in any compound described herein having one or more double bond(s) generating geometrical isomers that can be defined as E or Z, each double bond may independently be E or Z a mixture thereof. Likewise, it is understood that, in any compound described, all tautomeric forms are also intended to be included.

It is to be understood that where compounds disclosed herein have unfilled valencies, then the valencies are to be filled with hydrogens or isotopes thereof, e.g., hydrogen-1 (protium) and hydrogen-2 (deuterium).

It is understood that the compounds described herein can be labeled isotopically. Substitution with isotopes such as deuterium may afford certain therapeutic advantages resulting from greater metabolic stability, such as, for example, increased in vivo half-life or reduced dosage requirements. Each chemical element as represented in a compound structure may include any isotope of said element. For example, in a compound structure a hydrogen atom may be explicitly disclosed or understood to be present in the compound. At any position of the compound that a hydrogen atom may be present, the hydrogen atom can be any isotope of hydrogen, including but not limited to hydrogen-1 (protium) and hydrogen-2 (deuterium). Thus, reference herein to a compound encompasses all potential isotopic forms unless the context clearly dictates otherwise.

It is understood that the methods and combinations described herein include crystalline forms (also known as polymorphs, which include the different crystal packing arrangements of the same elemental composition of a compound), amorphous phases, salts, solvates and hydrates. In some embodiments, the compounds described herein exist in solvated forms with pharmaceutically acceptable solvents such as water, ethanol or the like. In other embodiments, the compounds described herein exist in unsolvated form. Solvates contain either stoichiometric or non-stoichiometric amounts of a solvent, and may be formed during the process of crystallization with pharmaceutically acceptable solvents such as water, ethanol or the like. Hydrates are formed when the solvent is water or alcoholates are formed when the solvent is alcohol. In addition, the compounds provided herein can exist in unsolvated as well as solvated forms. In general, the solvated forms are considered equivalent to the unsolvated forms for the purposes of the compounds and methods provided herein.

Where a range of values is provided, it is understood that the upper and lower limit, and each intervening value between the upper and lower limit of the range is encompassed within the embodiments.

Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide exemplary instances of the item in discussion, not an exhaustive or limiting list thereof; and use of terms like ‘preferably,’ ‘preferred,’ ‘desired,’ or ‘desirable,’ and words of similar meaning should not be understood as implying that certain features are critical, essential, or even important to the structure or function, but instead as merely intended to highlight alternative or additional features that may or may not be utilized in a particular embodiment. In addition, the term “comprising” is to be interpreted synonymously with the phrases “having at least” or “including at least”. When used in the context of a compound, composition or device, the term “comprising” means that the compound, composition or device includes at least the recited features or components, but may also include additional features or components.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

### Compounds

Some embodiments disclosed herein relate to a compound of Formula (I), or a pharmaceutically acceptable salt thereof, having the structure:

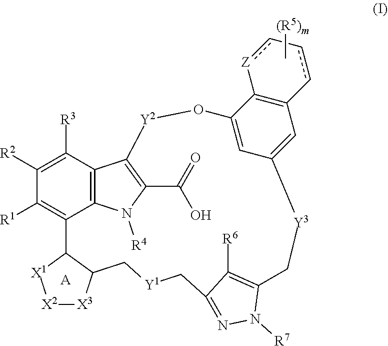

wherein: R¹, R², R³ and R⁶ can be each independently hydrogen, halogen, an unsubstituted C₁₋₄ alkyl or an unsubstituted C₁₋₄ haloalkyl; R⁴ and R⁷ can be each independently hydrogen, an optionally substituted C₁₋₄ alkyl, an optionally substituted C₃₋₆ monocyclic cycloalkyl or an unsubstituted C₁₋₄ haloalkyl; X¹, X² and X³ can be each independently NR⁸ or CR⁹; and wherein Ring A can be an aromatic ring; R⁸ and R⁹ can be each independently absent, hydrogen, halogen, cyano, an optionally substituted C₁₋₄ alkyl, an optionally substituted C₁₋₄ alkoxy, an optionally substituted C₃₋₆ monocyclic cycloalkyl, an optionally substituted C₃₋₆ bicyclic cycloalkyl, a mono-substituted amine or a di-substituted amine; or the substituent attached to X¹ and the substituted attached to X² can be taken together to form Ring B fused to Ring A; and X³ can be NR⁸ or CR⁹, and wherein Ring A and Ring B can form an optionally substituted heteroaryl or an optionally substituted heterocyclyl; or the substituent attached to X² and the substituted attached to X³ can be taken together to form Ring C fused to Ring A; and X¹ can be NR⁸ or CR⁹, and wherein Ring A and Ring C can form an optionally substituted heteroaryl or an optionally substituted heterocyclyl; Y¹ can be O (oxygen), S (sulfur), SO, SO₂, CH₂, CF₂ or NR^{10A}; Y² can be an optionally substituted C₁₋₄ alkylene, and when Y² can be substituted, each substituent can be independently halogen or an unsubstituted C₁₋₄ alkyl; Y³ can be O (oxygen), S (sulfur), SO, SO₂, CH₂, CF₂ or NR^{10B}; R^{10A} and R^{10B} can be independently hydrogen or an optionally substituted C₁₋₄ alkyl; Z can be NH or NCH₃; each [symbol] can be single bond; m can be 0, 1 or 2; and each R⁵ can be independently halogen or an optionally substituted C₁₋₄ alkyl.

The phenyl ring of the indole of Formula (I) can be unsubstituted or substituted. In some embodiments, R¹, R² and R³ can each be hydrogen. When the phenyl ring of the indole ring is substituted, the phenyl ring can be mono-, di- or tri-substituted. In some embodiments, R¹ can be halogen (such as fluoro or chloro). In other embodiments, R¹ can be an unsubstituted C₁₋₄ alkyl. Examples of unsubstituted C₁₋₄ alkyls include methyl, ethyl, n-propyl, isopropyl, n-butyl, isobutyl and tert-butyl. In still other embodiments, R¹ can be an unsubstituted C₁₋₄ haloalkyl, such as CF₃ and CHF₂. In some embodiments, R² can be hydrogen. In other embodiments, R² can be halogen, including those described herein. In still other embodiments, R² can be an unsubstituted C₁₋₄ alkyl, such as those described herein. In yet still other embodiments, R² can be an unsubstituted C₁₋₄ haloalkyl. In some embodiments, R³ can be hydrogen. In other embodiments, R³ can be halogen, such as F or Cl. In still other embodiments, R³ can be an unsubstituted C₁₋₄ alkyl (for example, methyl, ethyl, n-propyl, isopropyl, n-butyl, isobutyl and tert-butyl). In yet still other embodiments, R³ can be an unsubstituted C₁₋₄ haloalkyl. In some embodiments, R¹ can be halogen, an unsubstituted C₁₋₄ alkyl or an unsubstituted C₁₋₄ haloalkyl; and R² and R³ can be each hydrogen. In other embodiments, R¹ and R³ can be independently halogen, an unsubstituted C₁₋₄ alkyl or an unsubstituted C₁₋₄ haloalkyl; and R² can be hydrogen.

The 5-membered ring of the indole can be unsubstituted or substituted. In some embodiments, R⁴ can be hydrogen. In other embodiments, R⁴ can be an unsubstituted C₁₋₄ alkyl. In still other embodiments, R⁴ can be a substituted C₁₋₄ alkyl. Suitable C₁₋₄ alkyls are described herein and include methyl, ethyl, n-propyl, isopropyl, n-butyl, isobutyl and tert-butyl. In some embodiments, R⁴ can be an unsubstituted C₃₋₆ monocyclic cycloalkyl. In other embodiments, R⁴ can be a substituted C₃₋₆ monocyclic cycloalkyl. Examples of C₃₋₆ monocyclic cycloalkyls include cyclopropyl, cyclobutyl, cyclopentyl and cyclohexyl. In still other embodiments, R⁴ can be an unsubstituted C₁₋₄ haloalkyl, such as CHF₂ and CF₃.

The pyrazole of Formula (I),

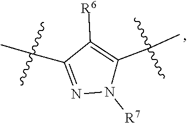

can be unsubstituted or substituted. When the pyrazole is unsubstituted, R⁶ and R⁷ can be each hydrogen. In some embodiments, the pyrazole can be substituted, wherein at least one of R⁶ and R⁷ is a non-hydrogen substituent. In some embodiments, R⁶ can be hydrogen. In other embodiments, R⁶ can be halogen. In still other embodiments, R⁶ can be an unsubstituted C₁₋₄ alkyl. In yet still other embodiments, R⁶ can be an unsubstituted C₁₋₄ haloalkyl. In some embodiments, R⁷ can be hydrogen. In other embodiments, R⁷ can be an unsubstituted C₁₋₄ alkyl. In still other embodiments, R⁷ can be a substituted C₁₋₄ alkyl. In yet still other embodiments, R⁷ can be an unsubstituted C₃₋₆ monocyclic cycloalkyl. In some embodiments, R⁷ can be a substituted C₃₋₆ monocyclic cycloalkyl. In other embodiments, R⁷ can be an unsubstituted C₁₋₄ haloalkyl. Examples of C₁₋₄ alkyl, C₃₋₆ monocyclic cycloalkyl and C₁₋₄ haloalkyls are described herein. Several examples of

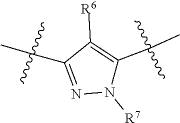

include the following:

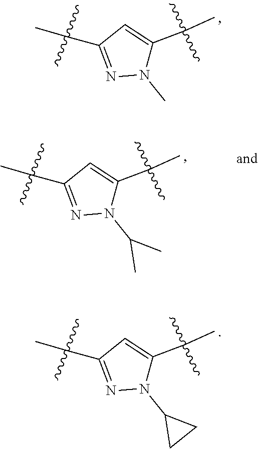

As described herein, Ring A can be a monocyclic aromatic ring, or when taken together with a second ring (such as Ring B or Ring C), Ring A together with the second ring can be an optionally substituted heteroaryl or an optionally substituted heterocyclyl. In some embodiments, X¹, X² and X³ can be each independently NR⁸ or CR⁹; and Ring A can be an aromatic ring, wherein R⁸ and R⁹ can be each independently absent, hydrogen, halogen, cyano, an optionally substituted C₁₋₄ alkyl, an optionally substituted C₁₋₄ alkoxy, an optionally substituted C₃₋₆ monocyclic cycloalkyl, an optionally substituted C₃₋₆ bicyclic cycloalkyl, a mono-substituted amine or a di-substituted amine. In some embodiments, at least one of X¹, X² and X³ is NR⁸. In some embodiments, X¹ can be CR⁹; and X² and X³ can be each NR⁸. In other embodiments, X¹ and X³ can be each CR⁹; and X² can be NR⁸. In still other embodiments, X¹ and X³ can be each NR⁸; and X² can be CR⁹. In yet still other embodiments, X¹ and X² can be each NR⁸; and X³ can be CR⁹. Various examples of Ring A being a monocyclic aromatic ring include the following:

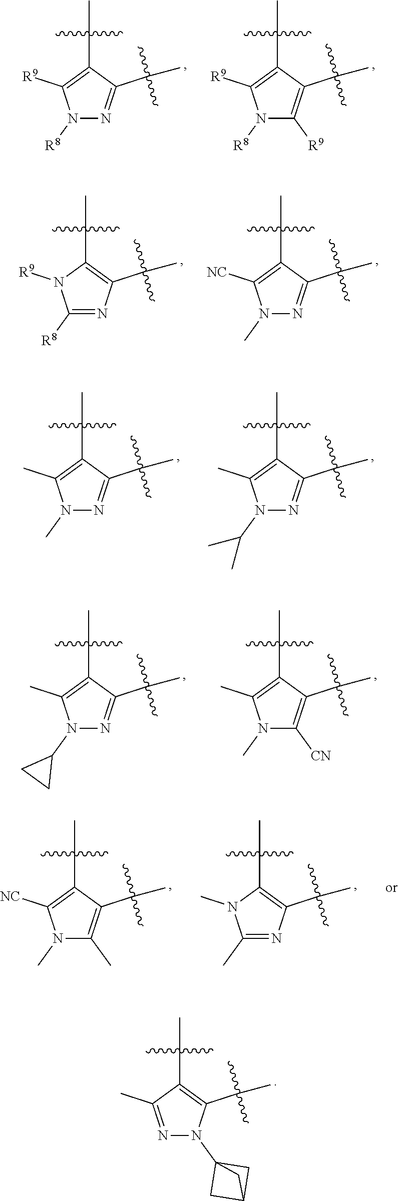

In other embodiments, X¹ and X² can be each independently NR⁸ or CR⁹; the substituent attached to X¹ and the substituted attached to X² can be taken together to form Ring B fused to Ring A; X³ can be NR⁸ or CR⁹; Ring A and Ring B can form an optionally substituted heteroaryl or an optionally substituted heterocyclyl; and R⁸ and R⁹ can be each independently absent, hydrogen, halogen, cyano, an optionally substituted C₁₋₄ alkyl, an optionally substituted C₁₋₄ alkoxy, an optionally substituted C₃₋₆ monocyclic cycloalkyl, an optionally substituted C₃₋₆ bicyclic cycloalkyl, a mono-substituted amine or a di-substituted amine. In some embodiments, X¹ and X² can be each independently NR⁸ or CR⁹; X³ can be NR⁸; and Ring A and Ring B can form an optionally substituted heteroaryl. In other embodiments, X¹ and X² can be each independently NR⁸ or CR⁹; X³ can be NR⁸; and Ring A and Ring B can form an optionally substituted heterocyclyl. In still other embodiments, X¹ and X² can be each independently NR⁸ or CR⁹; X³ can be CR⁹; and Ring A and Ring B can form an optionally substituted heteroaryl. In yet still other embodiments, X¹ and X² can be each independently NR⁸ or CR⁹; X³ can be CR⁹; and Ring A and Ring B can form an optionally substituted heterocyclyl. In some embodiments, X¹ can be CR⁹; X² can be NR⁸; X³ can be NR⁸; and Ring A and Ring B can form an optionally substituted heteroaryl. In other embodiments, X¹ can be CR⁹; X² can be NR⁸; X³ can be NR⁸; and Ring A and Ring B can form an optionally substituted heterocyclyl. Ring B can be a 5- to 6-membered ring.

Examples of the rings of this paragraph are:

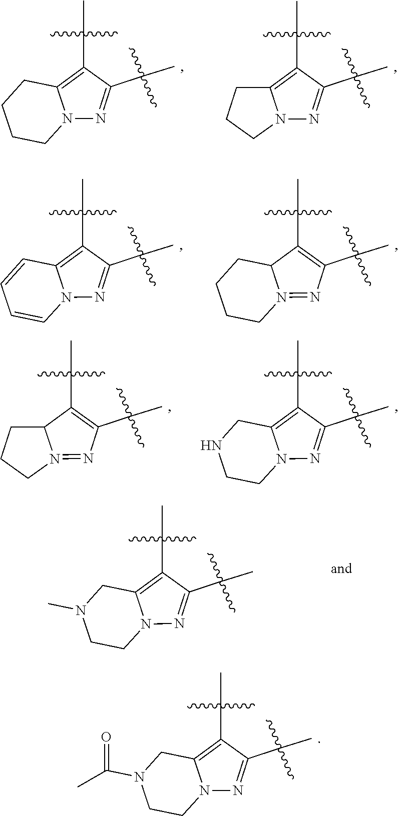

The aforementioned rings can be further substituted with substituents such as those described for “optionally substituted.”

In other embodiments, X² and X³ can be each independently NR⁸ or CR⁹; the substituent attached to X² and the substituted attached to X³ can be taken together to form Ring C fused to Ring A; X¹ can be NR⁸ or CR⁹; Ring A and Ring C can form an optionally substituted heteroaryl or an optionally substituted heterocyclyl; and R⁸ and R⁹ can be each independently absent, hydrogen, halogen, cyano, an optionally substituted C₁₋₄ alkyl, an optionally substituted C₁₋₄ alkoxy, an optionally substituted C₃₋₆ monocyclic cycloalkyl, an optionally substituted C₃₋₆ bicyclic cycloalkyl, a mono-substituted amine or a di-substituted amine. In some embodiments, X² and X³ can be each independently NR⁸ or CR⁹; X¹ can be NR⁸; and Ring A and Ring C can form an optionally substituted heteroaryl. In other embodiments, X² and X³ can be each independently NR⁸ or CR⁹; X¹ can be NR⁸; and Ring A and Ring C can form an optionally substituted heterocyclyl. In still other embodiments, X² and X³ can be each independently NR⁸ or CR⁹; X¹ can be CR⁹; and Ring A and Ring C can form an optionally substituted heteroaryl. In yet still other embodiments, X² and X³ can be each independently NR⁸ or CR⁹; X¹ can be CR⁹; and Ring A and Ring C can form an optionally substituted heterocyclyl. In some embodiments, X¹ can be CR⁹; X² can be NR⁸; X³ can be NR⁸; and Ring A and Ring C can form an optionally substituted heteroaryl. In other embodiments, X¹ can be CR⁹; X² can be NR⁸; X³ can be NR⁸; and Ring A and Ring C can form an optionally substituted heterocyclyl. Examples of the rings of this paragraph are:

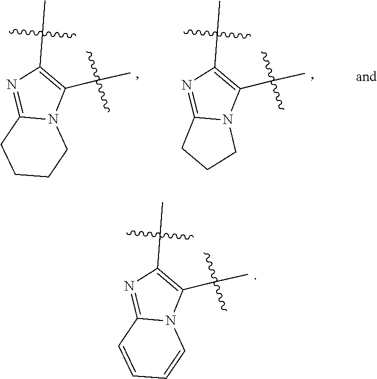 [SMILES: *c1nc2n(c1*)CCCC2 ; *c1nc2n(c1*)CCC2 ; *c1nc2ccccn2c1*]

These examples of rings can be further substituted with substituents such as those described for “optionally substituted.”

In some embodiments, each R⁸ and/or each R⁹ can be independently absent. In other embodiments, each R⁸ and/or each R⁹ can be independently hydrogen. In other embodiments, each R⁸ and/or each R⁹ can be independently cyano. In still other embodiments, each R⁸ and/or each R⁹ can be independently an unsubstituted C₁₋₄ alkyl (such as methyl, ethyl, n-propyl, isopropyl, n-butyl, isobutyl, sec-butyl or tert-butyl). In yet still other embodiments, each R⁸ and/or each R⁹ can be independently an unsubstituted C₁₋₄ alkoxy (such as methoxy, ethoxy, n-propoxy, isopropoxy, n-butoxy, isobutoxy, sec-butoxy or tert-butoxy). In some embodiments, each R⁸ and/or each R⁹ can be independently an unsubstituted C₃₋₆ monocyclic cycloalkyl, including cyclopropyl, cyclobutyl, cyclopentyl and cyclohexyl. In other embodiments, each R⁸ and/or each R⁹ can be independently an unsubstituted C₃₋₆ bicyclic cycloalkyl, for example, bicyclo[1.1.1]pentyl. In still other embodiments, each R⁸ and/or each R⁹ can be independently a mono-substituted amine. In yet still other embodiments, each R⁸ and/or each R⁹ can be independently a di-substituted amine. In some embodiments, each R⁸ and/or each R⁹ can be independently a substituted C₁₋₄ alkyl, a substituted C₁₋₄ alkoxy, a substituted C₃₋₆ monocyclic cycloalkyl, a substituted C₃₋₆ bicyclic cycloalkyl, a mono-substituted amine or a di-substituted amine. In some embodiments, each R⁸ can be independently hydrogen, an unsubstituted C₁₋₄ alkyl, an unsubstituted or a substituted C₃₋₆ monocyclic cycloalkyl or an unsubstituted or a substituted C₃₋₆ bicyclic cycloalkyl. In some embodiments, each R⁹ can be independently hydrogen, cyano, an unsubstituted C₁₋₄ alkyl.

In some embodiments, Z can be NH; and each [symbol] can be a single bond. In other embodiments, Z can be NCH₃; and each [symbol] can be a single bond. An examples of

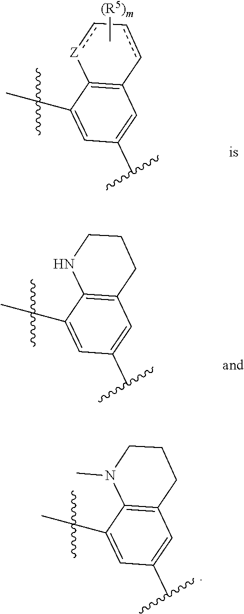

These examples of rings can be further substituted with substituents such as those described for “optionally substituted.”

In some embodiments, m can be 0, such that upper ring is unsubstituted. In other embodiments, m can be 1, wherein R⁵ can be halogen or an optionally substituted C₁₋₄ alkyl. In still other embodiments, m can be 2, wherein each R⁵ can be independently halogen or an optionally substituted C₁₋₄ alkyl. Suitable halogens (including fluoro and chloro) and an optionally substituted C₁₋₄ alkyls (optionally substituted versions of methyl, ethyl, n-propyl, isopropyl, n-butyl, isobutyl and tert-butyl). In some embodiments, each R⁵ can be independently an unsubstituted C₁₋₄ alkyl. In other embodiments, each R⁵ can be independently a substituted C₁₋₄ alkyl.

In some embodiments, Y¹ can be O (oxygen). In other embodiments, Y¹ can be S (sulfur). In still other embodiments, Y¹ can be SO. In yet still other embodiments, Y¹ can be SO₂. In some embodiments, Y¹ can be CH₂. In other embodiments, Y¹ can be CF₂. In other embodiments, Y¹ can be NR^{10A}, wherein R^{10A} can be hydrogen. In still other embodiments, Y¹ can be NR^{10A}, wherein R^{10A} can be an unsubstituted C₁₋₄ alkyl. In yet still other embodiments, Y¹ can be NR^{10A}, wherein R^{10A} can be a substituted C₁₋₄ alkyl. Examples of optionally substituted C₁₋₄ alkyls include substituted versions of methyl, ethyl, n-propyl, isopropyl, n-butyl, isobutyl or tert-butyl.

In some embodiments, Y² can be an unsubstituted C₁₋₄ alkylene. In other embodiments, Y² can be a substituted C₁₋₄ alkylene, wherein when Y² can be substituted, each substituent can be independently halogen or an unsubstituted C₁₋₄ alkyl. Exemplary optionally substituted C₁₋₄ alkylenes for Y² include: —CH₂—, —CH₂CH₂—, —CH₂CH₂CH₂—, —CH₂CH₂CH₂CH₂—, —CH(CH₃)CH₂CH₂—, —CHFCH₂CH₂— and —CH₂CF₂CH₂—.

In some embodiments, Y³ can be O (oxygen). In other embodiments, Y³ can be S (sulfur). In still other embodiments, Y³ can be SO. In yet still other embodiments, Y³ can be SO₂. In some embodiments, Y³ can be CH₂. In other embodiments, Y³ can be CF₂. In other embodiments, Y¹ can be NH. In still other embodiments, Y³ can be NR^{10B}, wherein R^{10B} can be an unsubstituted C₁₋₄ alkyl. In yet still other embodiments, Y³ can be NR^{10B}, wherein R^{10B} can be a substituted C₁₋₄ alkyl. Suitable optionally substituted C₁₋₄ alkyls include substituted versions of methyl, ethyl, n-propyl, isopropyl, n-butyl, isobutyl or tert-butyl.

In some embodiments, when Y¹, Y² and Y³ are: (1) Y¹ and Y³ are each S and Y² is —(CH₂)₃—; (2) Y¹ is S, Y² is —(CH₂)₃— and Y³ is —(CH₂)—; (3) Y¹ is NR^{10A}, Y² is —(CH₂)₃— and Y³ is S; or (4) Y¹ is NR^{10A}, Y² is —(CH₂)₃— and Y³ is —(CH₂)—; R¹ is chloro; R², R³ and R⁶ are each hydrogen; R⁴ and R⁷ are each methyl; Z is NH; each [symbol] is a single bond; and m is 0; then X¹, X² and X³ are not (1) X¹ is CR⁸, wherein R⁸ is an optionally substituted C₁₋₄ alkyl, X² is N and X³ is N(CH₃); and (2) X¹ is CR⁸, wherein R⁸ is an optionally substituted C₁₋₄ alkyl, X² is N(CH₃) and X³ is N.

In some embodiments, when Y¹ and Y³ are each S and Y² is —(CH₂)₃—; R¹ is chloro; R², R³ and R⁶ are each hydrogen; R⁴ and R⁷ are each methyl; Z is NH; each [symbol] is a single bond; and m is 0; then X¹, X² and X³ are not the following: X¹ is CR⁸, wherein R⁸ is an optionally substituted C₁₋₄ alkyl, X² is N and X³ is N(CH₃). In other embodiments, when Y¹ and Y³ are each S and Y² is —(CH₂)₃—; R¹ is chloro; R², R³ and R⁶ are each hydrogen; R⁴ and R⁷ are each methyl; Z is NH; each [symbol] is a single bond; and m is 0; then X¹, X² and X³ are not the following: X¹ is CR⁸, wherein R⁸ is an optionally substituted C₁₋₄ alkyl, X² is N(CH₃) and X³ is N.

In some embodiments, when Y¹ is S, Y² is —(CH₂)₃— and Y³ is —(CH₂)—; R¹ is chloro; R², R³ and R⁶ are each hydrogen; R⁴ and R⁷ are each methyl; Z is NH; each [symbol] is a single bond; and m is 0; then X¹, X² and X³ are not the following: X¹ is CR⁸, wherein R⁸ is an optionally substituted C₁₋₄ alkyl, X² is N and X³ is N(CH₃). In other embodiments, when Y¹ is S, Y² is —(CH₂)₃— and Y³ is —(CH₂)—; R¹ is chloro; R², R³ and R⁶ are each hydrogen; R⁴ and R⁷ are each methyl; Z is NH; each [symbol] is a single bond; and m is 0; then X¹, X² and X³ are not the following: X¹ is CR⁸, wherein R⁸ is an optionally substituted C₁₋₄ alkyl, X² is N(CH₃) and X³ is N.

In some embodiments, when Y¹ is NR^{10A}, Y² is —(CH₂)₃— and Y³ is S; R¹ is chloro; R², R³ and R⁶ are each hydrogen; R⁴ and R⁷ are each methyl; Z is NH; each [symbol] is a single bond; and m is 0; then X¹, X² and X³ are not the following: X¹ is CR⁸, wherein R⁸ is an optionally substituted C₁₋₄ alkyl, X² is N and X³ is N(CH₃). In other embodiments, when Y¹ is NR^{10A}, Y² is —(CH₂)₃— and Y³ is S; R¹ is chloro; R², R³ and R⁶ are each ydrogen; R⁴ and R⁷ are each methyl; Z is NH; each [symbol] is a single bond; and m is 0; then X¹, X² and X³ are not the following: X¹ is CR⁸, wherein R⁸ is an optionally substituted C₁₋₄ alkyl, X² is N(CH₃) and X³ is N.

In some embodiments, when Y¹ is NR^{10A}, Y² is —(CH₂)₃— and Y³ is —(CH₂)—; R¹ is chloro; R², R³ and R⁶ are each hydrogen; R⁴ and R⁷ are each methyl; Z is NH; each [symbol] is a single bond; and m is 0; then X¹, X² and X³ are not the following: X¹ is CR⁸, wherein R⁸ is an optionally substituted C₁₋₄ alkyl, X² is N and X³ is N(CH₃). In other embodiments, when Y¹ is NR^{10A}, Y² is —(CH₂)₃— and Y³ is —(CH₂)—; R¹ is chloro; R², R³ and R⁶ are each hydrogen; R⁴ and R⁷ are each methyl; Z is NH; each [symbol] is a single bond; and m is 0; then X¹, X² and X³ are not the following: X¹ is CR⁸, wherein R⁸ is an optionally substituted C₁₋₄ alkyl, X² is N(CH₃) and X³ is N.

In some embodiments, the indole of a compound of Formula (I), or a pharmaceutically acceptable salt thereof, cannot be

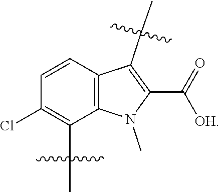 [SMILES: *c1c(C(=O)O)n(C)c2c(*)c(Cl)ccc12]

In some embodiments, Y² cannot be —(CH₂)₃—. In some embodiments, when Y¹ and Y³ are each S, then Y² cannot be —(CH₂)₃—. In other embodiments, when Y¹ is S and Y³ is —(CH₂)—, then Y² cannot be —(CH₂)₃—. In still other embodiments, when Y¹ is Y¹ is NR^{10A} and Y³ is —(CH₂)—, then Y² cannot be —(CH₂)₃—. In some embodiments, m cannot be 0. In some embodiments, when X¹ is CR⁸, wherein R⁸ is an optionally substituted C₁₋₄ alkyl, X² is N, then X³ cannot be N(CH₃). In some embodiments, when X¹ is CR⁸, wherein R⁸ is an optionally substituted C₁₋₄ alkyl, X² is N(CH₃), then X³ cannot be N (nitrogen). In some embodiments, the pyrazole of a compound of Formula (I), or a pharmaceutically acceptable salt thereof, cannot be

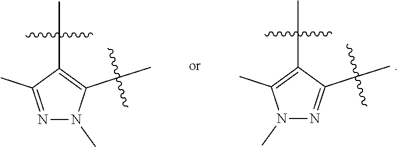

In some embodiments,

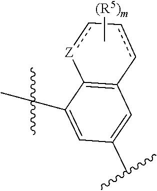

cannot be

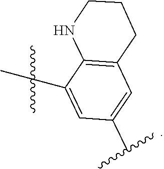 [SMILES: *c1cc(*)c2c(c1)CCCN2]

In some embodiments,

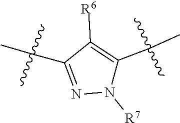

cannot be

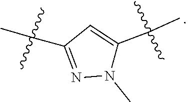 [SMILES: *c1cc(*)n(C)n1]

In some embodiments, R¹, R², R³ and R⁶ can be each independently hydrogen, halogen, an unsubstituted C₁₋₄ alkyl or an unsubstituted C₁₋₄ haloalkyl; R⁴ and R⁷ can be each independently hydrogen, an optionally substituted C₁₋₄ alkyl, an optionally substituted C₃₋₆ monocyclic cycloalkyl or an unsubstituted C₁₋₄ haloalkyl; X¹, X² and X³ can be each independently NR⁸ or CR⁹; and wherein Ring A can be an aromatic ring; R⁸ and R⁹ can be each independently absent, hydrogen, halogen, cyano, an optionally substituted C₁₋₄ alkyl, an optionally substituted C₁₋₄ alkoxy, an optionally substituted C₃₋₆ monocyclic cycloalkyl, an optionally substituted C₃₋₆ bicyclic cycloalkyl, a mono-substituted amine or a di-substituted amine; or the substituent attached to X¹ and the substituted attached to X² can be taken together to form Ring B fused to Ring A; and X³ can be NR⁸ or CR⁹, and wherein Ring A and Ring B can form an optionally substituted heteroaryl or an optionally substituted heterocyclyl; or the substituent attached to X² and the substituted attached to X³ can be taken together to form Ring C fused to Ring A; and X¹ can be NR⁸ or CR⁹, and wherein Ring A and Ring C can form an optionally substituted heteroaryl or an optionally substituted heterocyclyl; Y¹ can be O (oxygen), S (sulfur), SO, SO₂, CH₂, CF₂ or NR^{10A}; Y² can be an optionally substituted C₁₋₄ alkylene, and when Y² can be substituted, each substituent can be independently halogen or an unsubstituted C₁₋₄ alkyl; Y³ can be O (oxygen), S (sulfur), SO, SO₂, CH₂, CF₂ or NR^{10B}; R^{10A} and R^{10B} can be independently hydrogen or an optionally substituted C₁₋₄ alkyl; Z can be NH; each [symbol] can be single bond; m can be 0, 1 or 2; and each R⁵ can be independently halogen or an optionally substituted C₁₋₄ alkyl.

In some embodiments, a compound of Formula (I), or a pharmaceutically acceptable salt thereof, cannot a compound disclosed in WO 2018/178226 that would be encompassed by a compound of Formula (I), or a pharmaceutically acceptable salt thereof. In some embodiments, a compound of Formula (I), or a pharmaceutically acceptable salt thereof, cannot a compound disclosed in WO 2017/181625 that would be encompassed by a compound of Formula (I), or a pharmaceutically acceptable salt thereof.

Examples of compounds of Formula (I), and pharmaceutically acceptable salts thereof, include the following:

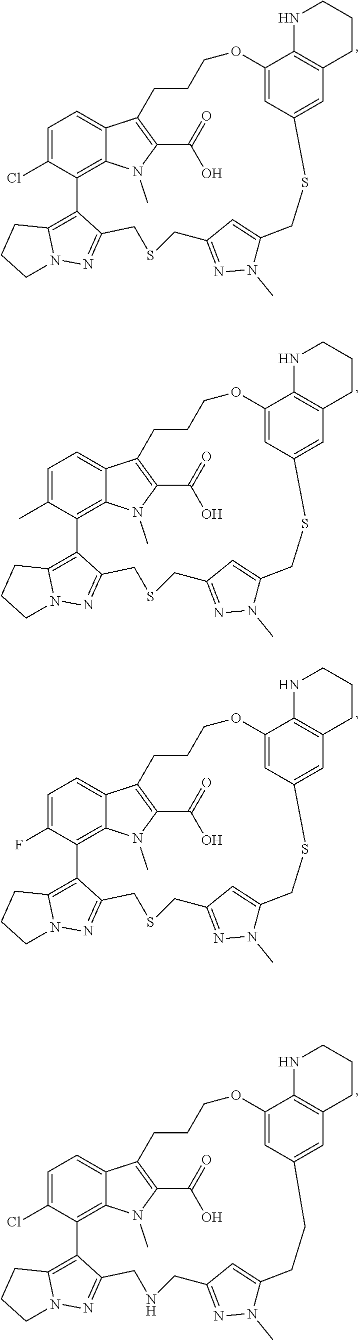 [SMILES: Cn1nc2cc1CSc1cc3c(c(c1)OCCCc1c(C(=O)O)n(C)c4c(c(Cl)ccc14)-c1c(nn4c1CCC4)CSC2)NCCC3 ; Cc1ccc2c3c(C(=O)O)n(C)c2c1-c1c(nn2c1CCC2)CSCc1cc(n(C)n1)CSc1cc2c(c(c1)OCCC3)NCCC2 ; Cn1nc2cc1CSc1cc3c(c(c1)OCCCc1c(C(=O)O)n(C)c4c(c(F)ccc14)-c1c(nn4c1CCC4)CSC2)NCCC3 ; Cn1nc2cc1CCc1cc3c(c(c1)OCCCc1c(C(=O)O)n(C)c4c(c(Cl)ccc14)-c1c(nn4c1CCC4)CNC2)NCCC3]

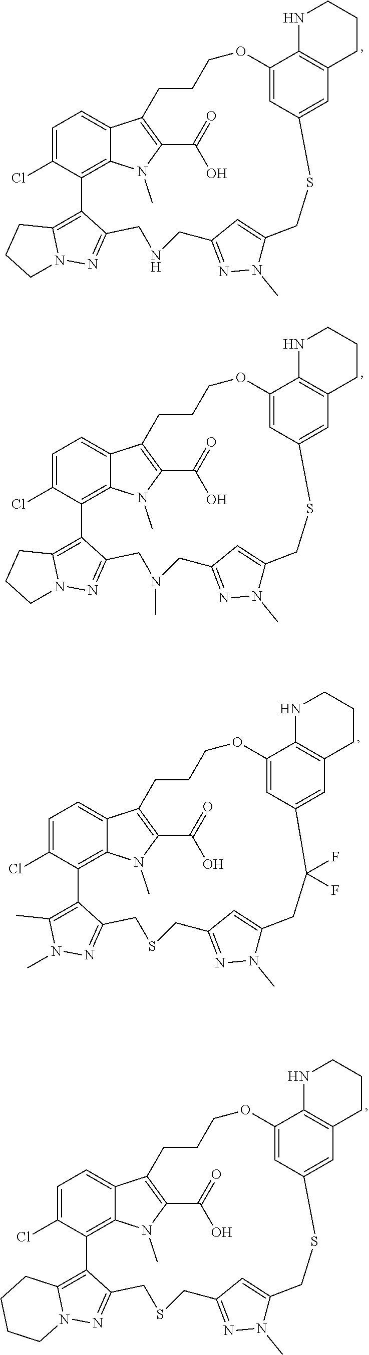 [SMILES: Cn1nc2cc1CSc1cc3c(c(c1)OCCCc1c(C(=O)O)n(C)c4c(c(Cl)ccc14)-c1c(nn4c1CCC4)CNC2)NCCC3 ; CN1Cc2cc(n(C)n2)CSc2cc3c(c(c2)OCCCc2c(C(=O)O)n(C)c4c(c(Cl)ccc24)-c2c(nn4c2CCC4)C1)NCCC3 ; Cc1c2c(nn1C)CSCc1cc(n(C)n1)CC(F)(F)c1cc3c(c(c1)OCCCc1c(C(=O)O)n(C)c4c-2c(Cl)ccc14)NCCC3 ; Cn1nc2cc1CSc1cc3c(c(c1)OCCCc1c(C(=O)O)n(C)c4c(c(Cl)ccc14)-c1c(nn4c1CCCC4)CSC2)NCCC3]

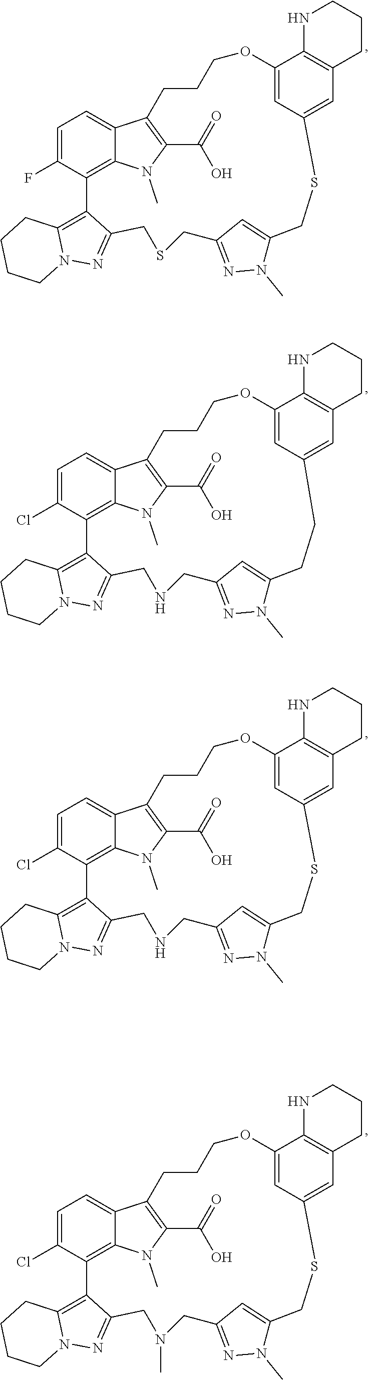 [SMILES: Cn1nc2cc1CSc1cc3c(c(c1)OCCCc1c(C(=O)O)n(C)c4c(c(F)ccc14)-c1c(nn4c1CCCC4)CSC2)NCCC3 ; Cn1nc2cc1CCc1cc3c(c(c1)OCCCc1c(C(=O)O)n(C)c4c(c(Cl)ccc14)-c1c(nn4c1CCCC4)CNC2)NCCC3 ; Cn1nc2cc1CSc1cc3c(c(c1)OCCCc1c(C(=O)O)n(C)c4c(c(Cl)ccc14)-c1c(nn4c1CCCC4)CNC2)NCCC3 ; CN1Cc2cc(n(C)n2)CSc2cc3c(c(c2)OCCCc2c(C(=O)O)n(C)c4c(c(Cl)ccc24)-c2c(nn4c2CCCC4)C1)NCCC3]

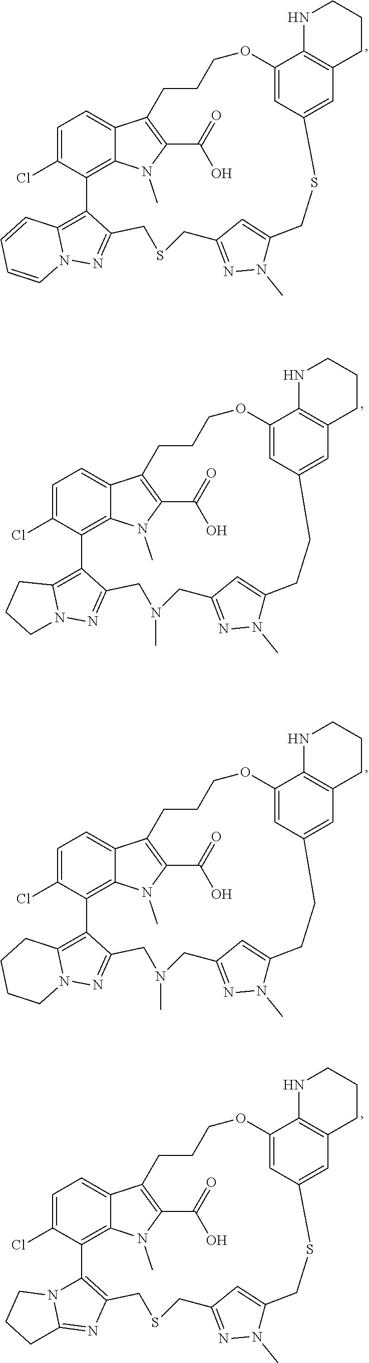 [SMILES: Cn1nc2cc1CSc1cc3c(c(c1)OCCCc1c(C(=O)O)n(C)c4c(c(Cl)ccc14)-c1c(nn4ccccc14)CSC2)NCCC3 ; CN1Cc2cc(n(C)n2)CCc2cc3c(c(c2)OCCCc2c(C(=O)O)n(C)c4c(c(Cl)ccc24)-c2c(nn4c2CCC4)C1)NCCC3 ; CN1Cc2cc(n(C)n2)CCc2cc3c(c(c2)OCCCc2c(C(=O)O)n(C)c4c(c(Cl)ccc24)-c2c(nn4c2CCCC4)C1)NCCC3 ; Cn1nc2cc1CSc1cc3c(c(c1)OCCCc1c(C(=O)O)n(C)c4c(c(Cl)ccc14)-c1c(nc4n1CCC4)CSC2)NCCC3]

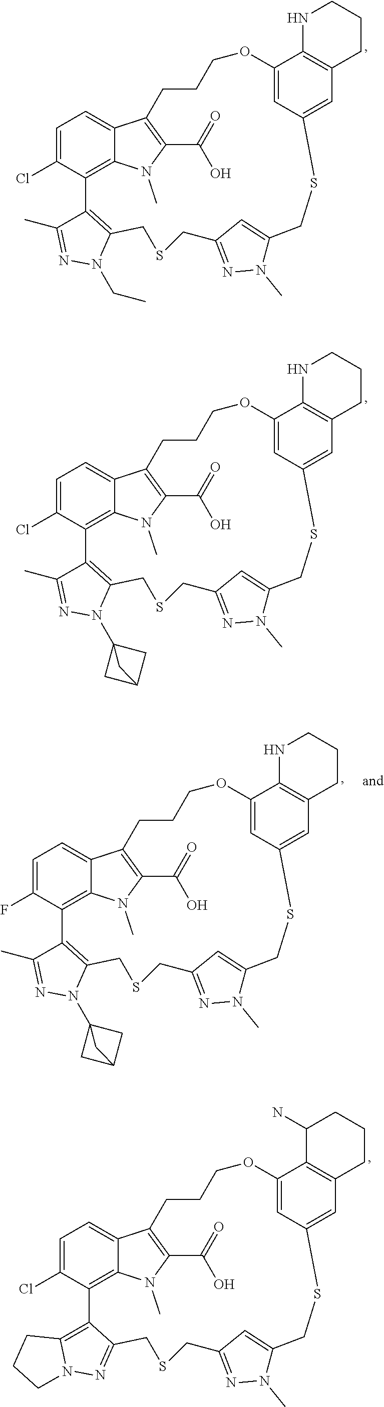 [SMILES: CCn1nc(C)c2c1CSCc1cc(n(C)n1)CSc1cc3c(c(c1)OCCCc1c(C(=O)O)n(C)c4c-2c(Cl)ccc14)NCCC3 ; Cc1nn(C23CC(C2)C3)c2c1-c1c(Cl)ccc3c(c(C(=O)O)n(C)c13)CCCOc1cc(cc3c1NCCC3)SCc1cc(nn1C)CSC2 ; Cc1nn(C23CC(C2)C3)c2c1-c1c(F)ccc3c(c(C(=O)O)n(C)c13)CCCOc1cc(cc3c1NCCC3)SCc1cc(nn1C)CSC2 ; Cn1nc2cc1CSc1cc3c(c(c1)OCCCc1c(C(=O)O)n(C)c4c(c(Cl)ccc14)-c1c(nn4c1CCC4)CSC2)C(N)CCC3]

or a pharmaceutically acceptable salt of any of the foregoing.

Additional examples of compounds of Formula (I), and pharmaceutically acceptable salts thereof, include the following:

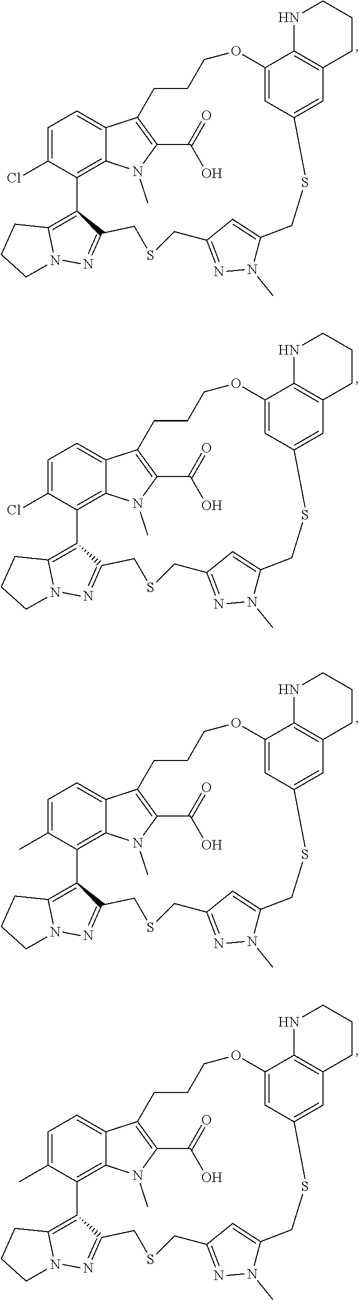

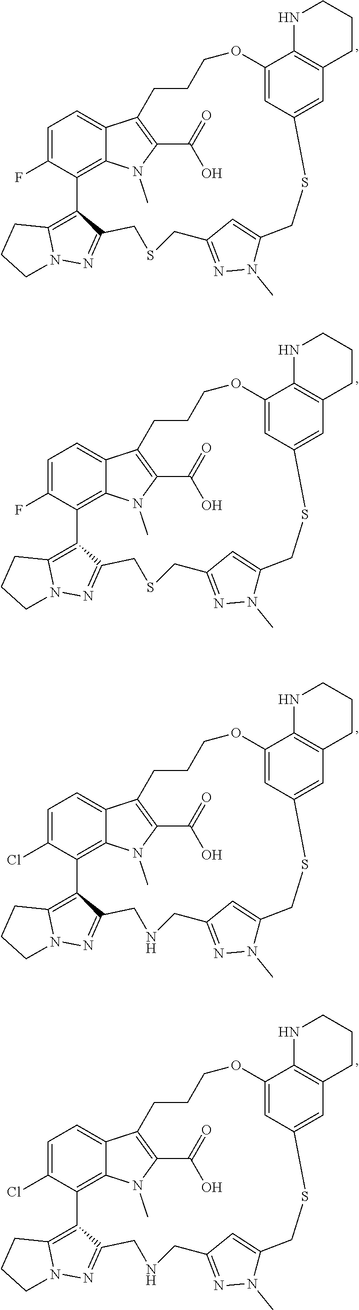

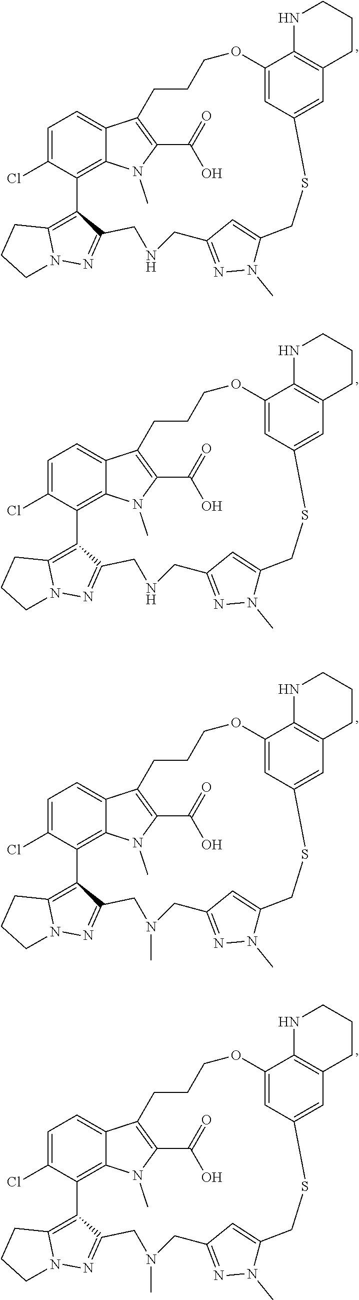

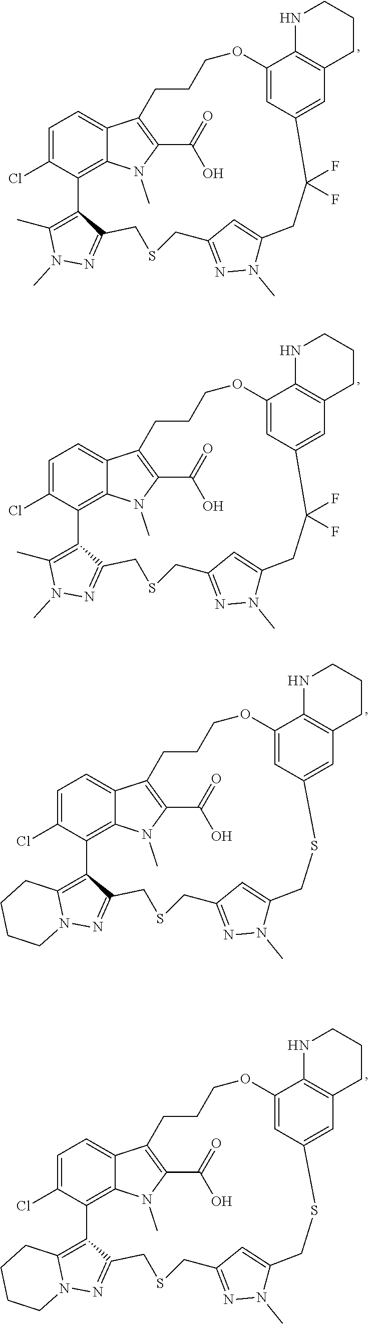

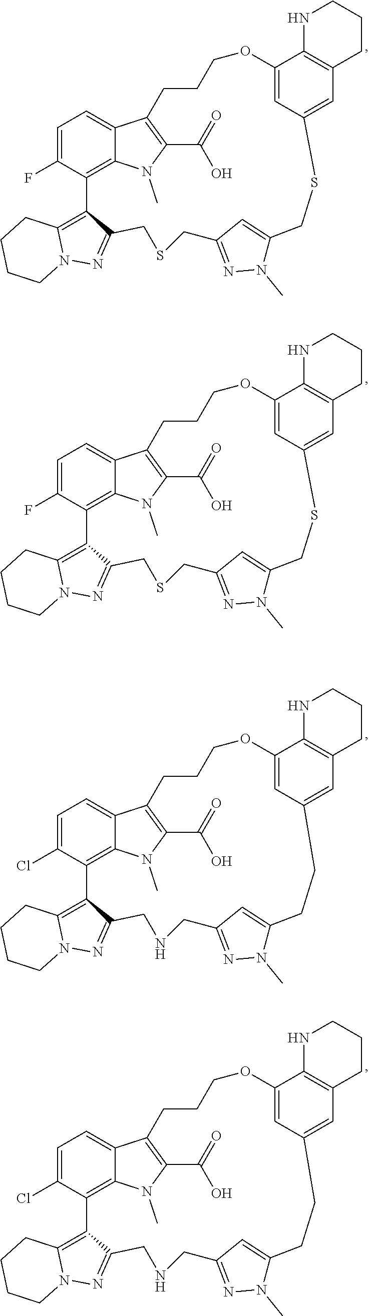

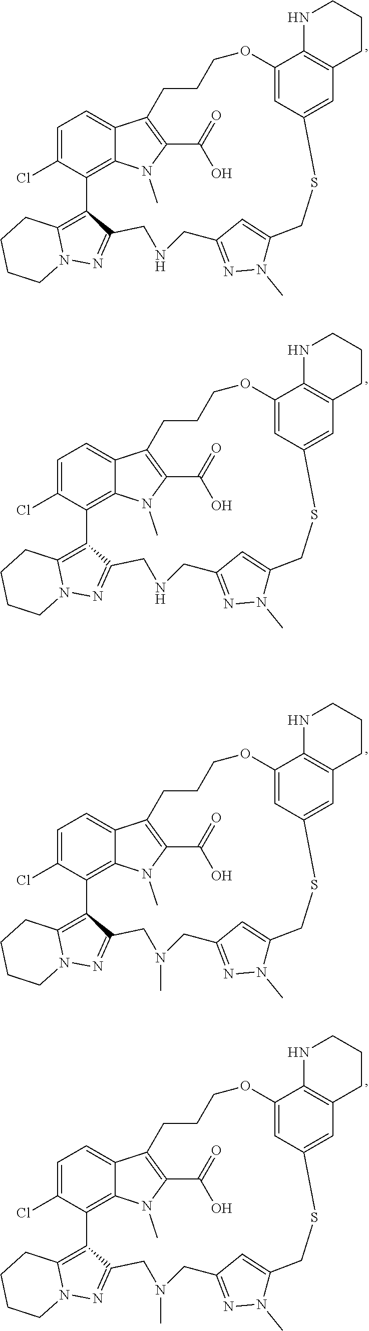

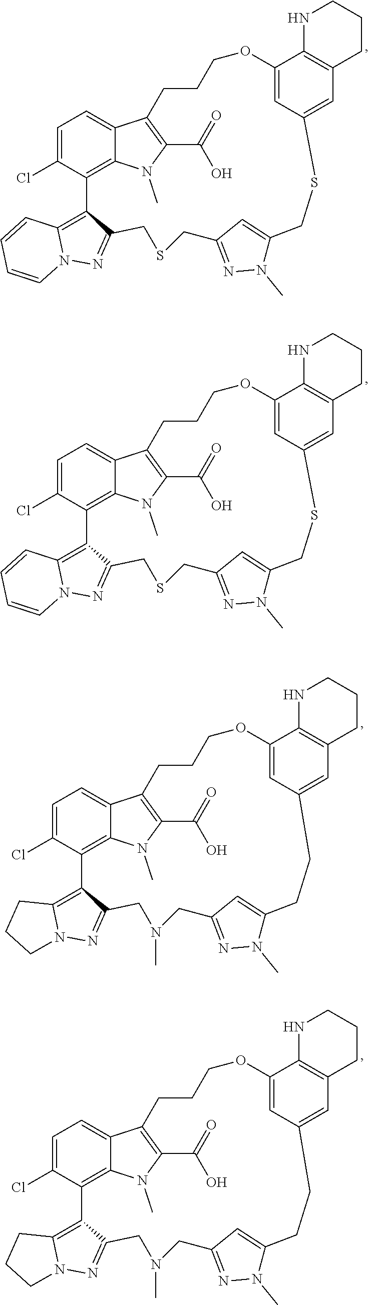

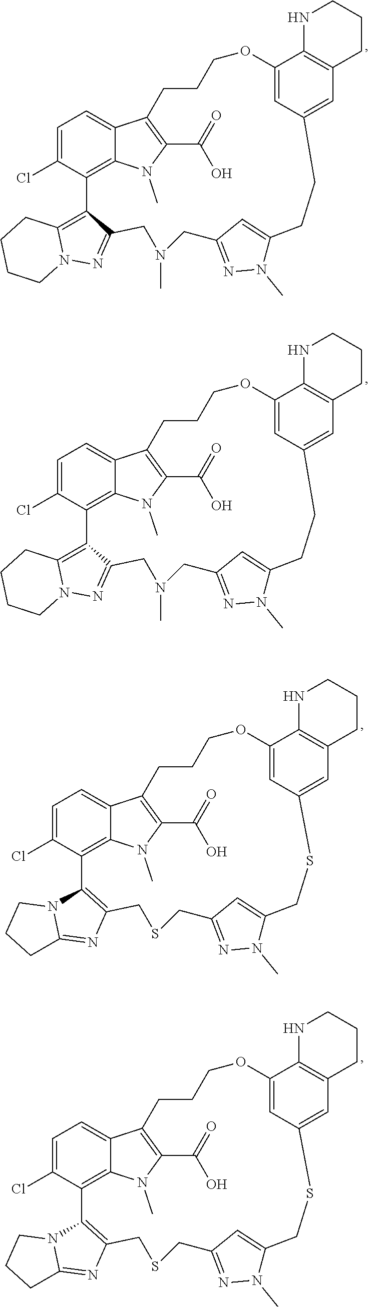

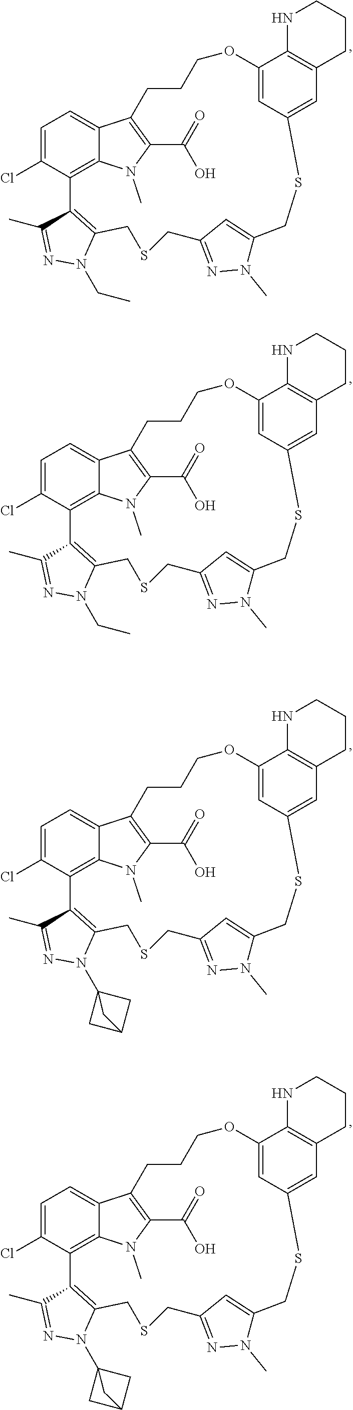

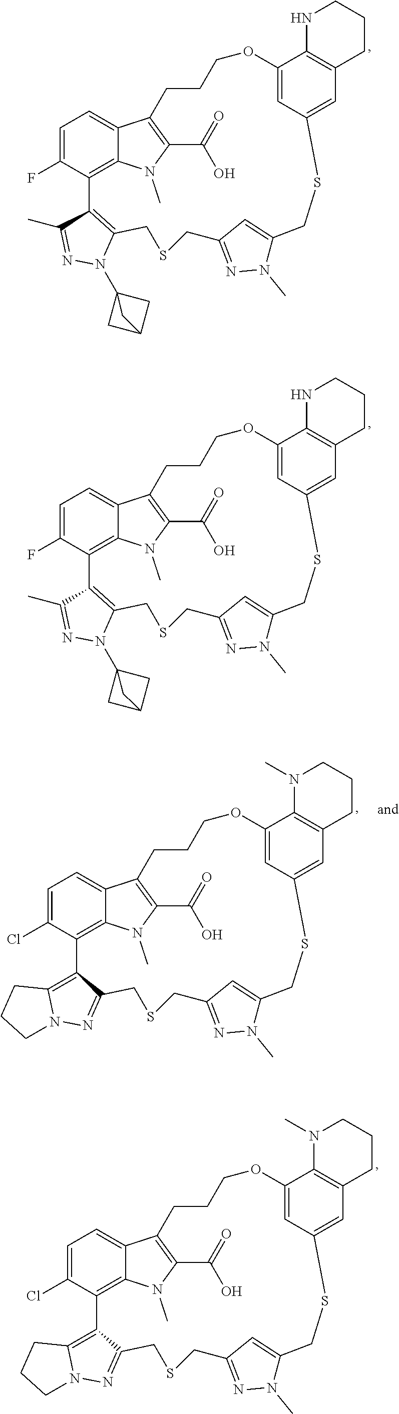

or a pharmaceutically acceptable salt of any of the foregoing.

### Synthesis

Compounds of the Formula (I), or pharmaceutically acceptable salts thereof, can be made in various ways by those skilled using known techniques as guided by the detailed teachings provided herein. For example, in an embodiment, compounds of the Formula (I) are prepared in accordance with General Scheme 1 as shown herein.

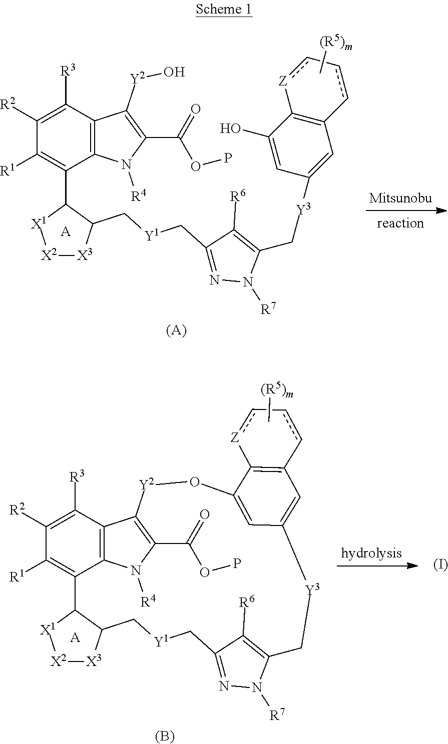

Compounds of Formula (I), and pharmaceutically acceptable salts thereof, can be prepared according to the preparation shown in Scheme 1. Compound A can undergo a Mitsunobu reaction and close the ring to form the macrocyclic Compound B. In Scheme 1, P represents a suitable protecting group. Removal of the protecting group via a hydrolysis reaction provides a compound of Formula (I), or a pharmaceutically acceptable salt thereof.

### Pharmaceutical Compositions

Some embodiments described herein relate to a pharmaceutical composition, that can include an effective amount of one or more compounds described herein (for example, a compound of Formula (I), or a pharmaceutically acceptable salt thereof) and a pharmaceutically acceptable carrier, diluent, excipient or combination thereof.

The term “pharmaceutical composition” refers to a mixture of one or more compounds and/or salts disclosed herein with other chemical components, such as diluents or carriers. The pharmaceutical composition facilitates administration of the compound to an organism. Pharmaceutical compositions can also be obtained by reacting compounds with inorganic or organic acids such as hydrochloric acid, hydrobromic acid, sulfuric acid, nitric acid, phosphoric acid, methanesulfonic acid, ethanesulfonic acid, p-toluenesulfonic acid, and salicylic acid. Pharmaceutical compositions will generally be tailored to the specific intended route of administration.

The term “physiologically acceptable” defines a carrier, diluent or excipient that does not abrogate the biological activity and properties of the compound nor cause appreciable damage or injury to an animal to which delivery of the composition is intended.

As used herein, a “carrier” refers to a compound that facilitates the incorporation of a compound into cells or tissues. For example, without limitation, dimethyl sulfoxide (DMSO) is a commonly utilized carrier that facilitates the uptake of many organic compounds into cells or tissues of a subject.

As used herein, a “diluent” refers to an ingredient in a pharmaceutical composition that lacks appreciable pharmacological activity but may be pharmaceutically necessary or desirable. For example, a diluent may be used to increase the bulk of a potent drug whose mass is too small for manufacture and/or administration. It may also be a liquid for the dissolution of a drug to be administered by injection, ingestion or inhalation. A common form of diluent in the art is a buffered aqueous solution such as, without limitation, phosphate buffered saline that mimics the pH and isotonicity of human blood.

As used herein, an “excipient” refers to an essentially inert substance that is added to a pharmaceutical composition to provide, without limitation, bulk, consistency, stability, binding ability, lubrication, disintegrating ability etc., to the composition. For example, stabilizers such as anti-oxidants and metal-chelating agents are excipients. In an embodiment, the pharmaceutical composition comprises an anti-oxidant and/or a metal-chelating agent. A “diluent” is a type of excipient.

The pharmaceutical compositions described herein can be administered to a human patient per se, or in pharmaceutical compositions where they are mixed with other active ingredients, as in combination therapy, or carriers, diluents, excipients or combinations thereof. Proper formulation is dependent upon the route of administration chosen. Techniques for formulation and administration of the compounds described herein are known to those skilled in the art.

The pharmaceutical compositions disclosed herein may be manufactured in a manner that is itself known, e.g., by means of conventional mixing, dissolving, granulating, dragee-making, levigating, emulsifying, encapsulating, entrapping or tableting processes. Additionally, the active ingredients are contained in an amount effective to achieve its intended purpose. Many of the compounds used in the pharmaceutical combinations disclosed herein may be provided as salts with pharmaceutically compatible counterions.

Multiple techniques of administering a compound, salt and/or composition exist in the art including, but not limited to, oral, rectal, pulmonary, topical, aerosol, injection, infusion and parenteral delivery, including intramuscular, subcutaneous, intravenous, intramedullary injections, intrathecal, direct intraventricular, intraperitoneal, intranasal and intraocular injections. In some embodiments, a compound of Formula (I), or a pharmaceutically acceptable salt thereof, can be administered orally.

One may also administer the compound, salt and/or composition in a local rather than systemic manner, for example, via injection or implantation of the compound directly into the affected area, often in a depot or sustained release formulation. Furthermore, one may administer the compound in a targeted drug delivery system, for example, in a liposome coated with a tissue-specific antibody. The liposomes will be targeted to and taken up selectively by the organ. For example, intranasal or pulmonary delivery to target a respiratory disease or condition may be desirable.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions that can include a compound and/or salt described herein formulated in a compatible pharmaceutical carrier may also be prepared, placed in an appropriate container, and labeled for treatment of an indicated condition.

### Uses and Methods of Treatment

Some embodiments described herein relate to a method for ameliorating and/or treating a cancer described herein that can include administering an effective amount of a compound described herein (for example, a compound of Formula (I), or a pharmaceutically acceptable salt thereof) or a pharmaceutical composition that includes an effective amount of a compound described herein (for example, a compound of Formula (I), or a pharmaceutically acceptable salt thereof) to a subject having a cancer described herein. Other embodiments described herein relate to the use of an effective amount of a compound described herein (for example, a compound of Formula (I), or a pharmaceutically acceptable salt thereof) or a pharmaceutical composition that includes an effective amount of a compound described herein (for example, a compound of Formula (I), or a pharmaceutically acceptable salt thereof) in the manufacture of a medicament for ameliorating and/or treating a cancer described herein. Still other embodiments described herein relate to an effective amount of a compound described herein (for example, a compound of Formula (I), or a pharmaceutically acceptable salt thereof) or a pharmaceutical composition that includes an effective amount of a compound described herein (for example, a compound of Formula (I), or a pharmaceutically acceptable salt thereof) for ameliorating and/or treating a cancer described herein.

Some embodiments described herein relate to a method for inhibiting replication of a malignant growth or a tumor that can include contacting the growth or the tumor with an effective amount of a compound described herein (for example, a compound of Formula (I), or a pharmaceutically acceptable salt thereof) or a pharmaceutical composition that includes an effective amount of a compound described herein (for example, a compound of Formula (I), or a pharmaceutically acceptable salt thereof), wherein the malignant growth or tumor is due to a cancer described herein. Other embodiments described herein relate to the use of an effective amount of a compound described herein (for example, a compound of Formula (I), or a pharmaceutically acceptable salt thereof) or a pharmaceutical composition that includes an effective amount of a compound described herein (for example, a compound of Formula (I), or a pharmaceutically acceptable salt thereof) in the manufacture of a medicament for inhibiting replication of a malignant growth or a tumor, wherein the malignant growth or tumor is due to a cancer described herein. Still other embodiments described herein relate to an effective amount of a compound described herein (for example, a compound of Formula (I), or a pharmaceutically acceptable salt thereof) or a pharmaceutical composition that includes an effective amount of a compound described herein (for example, a compound of Formula (I), or a pharmaceutically acceptable salt thereof) for inhibiting replication of a malignant growth or a tumor, wherein the malignant growth or tumor is due to a cancer described herein.

Some embodiments described herein relate to a method for ameliorating or treating a cancer described herein that can include contacting a malignant growth or a tumor with an effective amount of a compound described herein (for example, a compound of Formula (I), or a pharmaceutically acceptable salt thereof) or a pharmaceutical composition that includes an effective amount of a compound described herein (for example, a compound of Formula (I), or a pharmaceutically acceptable salt thereof) to a subject having a cancer described herein. Other embodiments described herein relate to the use of an effective amount of a compound described herein (for example, a compound of Formula (I), or a pharmaceutically acceptable salt thereof) or a pharmaceutical composition that includes an effective amount of a compound described herein (for example, a compound of Formula (I), or a pharmaceutically acceptable salt thereof) in the manufacture of a medicament for ameliorating or treating a cancer that can include contacting a malignant growth or a tumor, wherein the malignant growth or tumor is due to a cancer described herein. Still other embodiments described herein relate to an effective amount of a compound described herein (for example, a compound of Formula (I), or a pharmaceutically acceptable salt thereof) or a pharmaceutical composition that includes an effective amount of a compound described herein (for example, a compound of Formula (I), or a pharmaceutically acceptable salt thereof) for ameliorating or treating a cancer that can include contacting a malignant growth or a tumor, wherein the malignant growth or tumor is due to a cancer described herein.

Some embodiments described herein relate to a method for inhibiting the activity of Mcl-1 that can include providing an effective amount of a compound described herein (for example, a compound of Formula (I), or a pharmaceutically acceptable salt thereof) or a pharmaceutical composition that includes an effective amount of a compound described herein (for example, a compound of Formula (I), or a pharmaceutically acceptable salt thereof) to a cancer cell from a cancer described herein. Other embodiments described herein relate to the use of an effective amount of a compound described herein (for example, a compound of Formula (I), or a pharmaceutically acceptable salt thereof) or a pharmaceutical composition that includes an effective amount of a compound described herein (for example, a compound of Formula (I), or a pharmaceutically acceptable salt thereof) in the manufacture of a medicament for inhibiting the activity of Mcl-1. Still other embodiments described herein relate to an effective amount of a compound described herein (for example, a compound of Formula (I), or a pharmaceutically acceptable salt thereof) or a pharmaceutical composition that includes an effective amount of a compound described herein (for example, a compound of Formula (I), or a pharmaceutically acceptable salt thereof) for inhibiting the activity of Mcl-1. Some embodiments described herein relate to a method for inhibiting the activity of Mcl-1 that can include providing an effective amount of a compound described herein (for example, a compound of Formula (I), or a pharmaceutically acceptable salt thereof) or a pharmaceutical composition that includes an effective amount of a compound described herein (for example, a compound of Formula (I), or a pharmaceutically acceptable salt thereof) to a cancer cell from a cancer described herein. Other embodiments described herein relate to a method for inhibiting the activity of Mcl-1 that can include contacting a cancer cell from a cancer described herein with an effective amount of a compound described herein (for example, a compound of Formula (I), or a pharmaceutically acceptable salt thereof) or a pharmaceutical composition that includes an effective amount of a compound described herein (for example, a compound of Formula (I), or a pharmaceutically acceptable salt thereof), and thereby inhibiting the activity of Mcl-1.

Some embodiments described herein relate to a method for ameliorating or treating a cancer described herein that can include inhibiting the activity of Mcl-1 using an effective amount of a compound described herein (for example, a compound of Formula (I), or a pharmaceutically acceptable salt thereof) or a pharmaceutical composition that includes an effective amount of a compound described herein (for example, a compound of Formula (I), or a pharmaceutically acceptable salt thereof). Other embodiments described herein relate to the use of an effective amount of a compound described herein (for example, a compound of Formula (I), or a pharmaceutically acceptable salt thereof) or a pharmaceutical composition that includes an effective amount of a compound described herein (for example, a compound of Formula (I), or a pharmaceutically acceptable salt thereof) in the manufacture of a medicament for ameliorating or treating a cancer described herein by inhibiting the activity of Mcl-1. Still other embodiments described herein relate to an effective amount of a compound described herein (for example, a compound of Formula (I), or a pharmaceutically acceptable salt thereof) or a pharmaceutical composition that includes an effective amount of a compound described herein (for example, a compound of Formula (I), or a pharmaceutically acceptable salt thereof) for ameliorating or treating a cancer described herein by inhibiting the activity of Mcl-1. Some embodiments described herein relate to a method for ameliorating or treating a cancer described herein that can include contacting a cancer cell with an effective amount of a compound described herein (for example, a compound of Formula (I), or a pharmaceutically acceptable salt thereof) or a pharmaceutical composition that includes an effective amount of a compound described herein (for example, a compound of Formula (I), or a pharmaceutically acceptable salt thereof), wherein the compound inhibits the activity of Mcl-1.

Some embodiments disclosed herein relate to a method for inhibiting the activity of Mcl-1 that can include providing an effective amount of a compound described herein (for example, a compound of Formula (I), or a pharmaceutically acceptable salt thereof) or a pharmaceutical composition that includes an effective amount of a compound described herein (for example, a compound of Formula (I), or a pharmaceutically acceptable salt thereof) to a subject having a cancer described herein or a cancer cell from a cancer described herein. Other embodiments disclosed herein relate to the use of an effective amount of a compound described herein (for example, a compound of Formula (I), or a pharmaceutically acceptable salt thereof) or a pharmaceutical composition that includes an effective amount of a compound described herein (for example, a compound of Formula (I), or a pharmaceutically acceptable salt thereof) in the manufacture of a medicament for inhibiting the activity of Mcl-1. Still other embodiments disclosed herein relate to a compound described herein (for example, a compound of Formula (I), or a pharmaceutically acceptable salt thereof) or a pharmaceutical composition that includes an effective amount of a compound described herein (for example, a compound of Formula (I), or a pharmaceutically acceptable salt thereof) for inhibiting the activity of Mcl-1.

Examples of suitable cancers include, but are not limited to: hematological malignancies (such as acute myeloid leukemia, multiple myeloma, mantle cell lymphoma, chronic lymphocytic leukemia, diffuse large B cell lymphoma, Burkitt's lymphoma, follicular lymphoma) and solid tumors, for example, non-small cell lung cancer (NSCLC), small cell lung cancer (SCLC), breast cancer, neuroblastoma, prostate cancer, melanoma, pancreatic cancer, uterine, endometrial, colon, oesophagus and liver cancers, osteosarcoma, Hodgkin lymphoma, mesothelioma, meningioma, glioma and tumors of upper aerodigestive, ovarian, thyroid, stomach and urinary tract.

As described herein, a cancer can become resistant to one or more anti-cancer agents. In some embodiments, a compound described herein (for example, a compound of Formula (I), or a pharmaceutically acceptable salt thereof) or a pharmaceutical composition that includes an effective amount of a compound described herein (for example, a compound of Formula (I), or a pharmaceutically acceptable salt thereof) can be used to treat and/or ameliorate a cancer that has become resistant to one or more anti-cancer agents (such as one or more Mcl-1 inhibitors). Examples of anti-cancer agents that a subject may have developed resistance to include, but are not limited to, Mcl-1 inhibitors (such as AT101, gambogic acid, TW-37, AZD5991, Sabutoclax (BI-97C1), Maritoclax, UMI-77, A4210477. S63845, MIK665/S64315, (−)BI97D6 and/or AMG176). In some embodiments, the cancer that has become resistant to one or more anti-cancer agents can be a cancer described herein.

Several known Mcl-1 inhibitors can cause one or more undesirable side effects in the subject being treated. Examples of undesirable side effects include, but are not limited to, thrombocytopenia, neutropenia, anemia, diarrhea, vomiting, nausea, abdominal pain, and constipation. In some embodiments, a compound described herein (for example, a compound of Formula (I), or a pharmaceutically acceptable salt thereof) can decrease the number and/or severity of one or more side effects associated with a known Mcl-1 inhibitor. In some embodiments, a compound of Formula (I), or a pharmaceutically acceptable salt thereof, can result in a severity of a side effect (such as one of those described herein) that is 25% less than compared to the severity of the same side effect experienced by a subject receiving a known Mcl-1 inhibitor (such as AT101, gambogic acid, TW-37, AZD5991, Sabutoclax (BI-97C1), Maritoclax, UMI-77, A-1210477, S63845, M1K665/S64315, (−)B197D6 and/or AMG176). In some embodiments, a compound of Formula (I), or a pharmaceutically acceptable salt thereof, results in a number of side effects that is 25% less than compared to the number of side effects experienced by a subject receiving a known Mcl-1. In some embodiments, a compound of Formula (I), or a pharmaceutically acceptable salt thereof, results in a severity of a side effect (such as one of those described herein) that is less in the range of about 10% to about 30% compared to the severity of the same side effect experienced by a subject receiving a known Mcl-1. In some embodiments, a compound of Formula (I), or a pharmaceutically acceptable salt thereof, results in a number of side effects that is in the range of about 10% to about 30% less than compared to the number of side effects experienced by a subject receiving a known Mcl-1.

The one or more compounds of Formula (I), or a pharmaceutically acceptable salt thereof, that can be used to treat, ameliorate and/or inhibit the growth of a cancer wherein inhibiting the activity of Mcl-1 is beneficial is provided in any of the embodiments described in paragraphs [0064]-[0084], under the heading titled “Compounds.”

As used herein, a “subject” refers to an animal that is the object of treatment, observation or experiment. “Animal” includes cold- and warm-blooded vertebrates and invertebrates such as fish, shellfish, reptiles and, in particular, mammals. “Mammal” includes, without limitation, mice, rats, rabbits, guinea pigs, dogs, cats, sheep, goats, cows, horses, primates, such as monkeys, chimpanzees, and apes, and, in particular, humans. In some embodiments, the subject can be human. In some embodiments, the subject can be a child and/or an infant, for example, a child or infant with a fever. In other embodiments, the subject can be an adult.

As used herein, the terms “treat,” “treating,” “treatment,” “therapeutic,” and “therapy” do not necessarily mean total cure or abolition of the disease or condition. Any alleviation of any undesired signs or symptoms of the disease or condition, to any extent can be considered treatment and/or therapy. Furthermore, treatment may include acts that may worsen the subject's overall feeling of well-being or appearance.

The terms “therapeutically effective amount” and “effective amount” are used to indicate an amount of an active compound, or pharmaceutical agent, that elicits the biological or medicinal response indicated. For example, a therapeutically effective amount of compound, salt or composition can be the amount needed to prevent, alleviate or ameliorate symptoms of the disease or condition, or prolong the survival of the subject being treated. This response may occur in a tissue, system, animal or human and includes alleviation of the signs or symptoms of the disease or condition being treated. Determination of an effective amount is well within the capability of those skilled in the art, in view of the disclosure provided herein. The therapeutically effective amount of the compounds disclosed herein required as a dose will depend on the route of administration, the type of animal, including human, being treated and the physical characteristics of the specific animal under consideration. The dose can be tailored to achieve a desired effect, but will depend on such factors as weight, diet, concurrent medication and other factors which those skilled in the medical arts will recognize.

For example, an effective amount of a compound, or radiation, is the amount that results in: (a) the reduction, alleviation or disappearance of one or more symptoms caused by the cancer, (b) the reduction of tumor size, (c) the elimination of the tumor, and/or (d) long-term disease stabilization (growth arrest) of the tumor. In the treatment of lung cancer (such as non-small cell lung cancer) a therapeutically effective amount is that amount that alleviates or eliminates cough, shortness of breath and/or pain. As another example, an effective amount, or a therapeutically effective amount of a Mcl-1 inhibitor is the amount which results in the reduction in Mcl-1 activity and/or phosphorylation (such as phosphorylation of CDC₂). The reduction in Mcl-1 activity is known to those skilled in the art and can be determined by the analysis of Mcl-1 intrinsic kinase activity and downstream substrate phosphorylation.

The amount of the compound of Formula (I), or a pharmaceutically acceptable salt thereof, required for use in treatment will vary not only with the particular compound or salt selected but also with the route of administration, the nature and/or symptoms of the disease or condition being treated and the age and condition of the patient and will be ultimately at the discretion of the attendant physician or clinician. In cases of administration of a pharmaceutically acceptable salt, dosages may be calculated as the free base. As will be understood by those of skill in the art, in certain situations it may be necessary to administer the compounds disclosed herein in amounts that exceed, or even far exceed, the dosage ranges described herein in order to effectively and aggressively treat particularly aggressive diseases or conditions.

In general, however, a suitable dose will often be in the range of from about 0.05 mg/kg to about 10 mg/kg. For example, a suitable dose may be in the range from about 0.10 mg/kg to about 7.5 mg/kg of body weight per day, such as about 0.15 mg/kg to about 5.0 mg/kg of body weight of the recipient per day, about 0.2 mg/kg to 4.0 mg/kg of body weight of the recipient per day, or any amount in between. The compound may be administered in unit dosage form; for example, containing 1 to 500 mg, 10 to 100 mg, 5 to 50 mg or any amount in between, of active ingredient per unit dosage form.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations.

As will be readily apparent to one skilled in the art, the useful in vivo dosage to be administered and the particular mode of administration will vary depending upon the age, weight, the severity of the affliction, the mammalian species treated, the particular compounds employed and the specific use for which these compounds are employed. The determination of effective dosage levels, that is the dosage levels necessary to achieve the desired result, can be accomplished by one skilled in the art using routine methods, for example, human clinical trials, in vivo studies and in vitro studies. For example, useful dosages of a compound of Formula (I), or pharmaceutically acceptable salts thereof, can be determined by comparing their in vitro activity, and in vivo activity in animal models. Such comparison can be done by comparison against an established drug, such as cisplatin and/or gemcitabine)

Dosage amount and interval may be adjusted individually to provide plasma levels of the active moiety which are sufficient to maintain the modulating effects, or minimal effective concentration (MEC). The MEC will vary for each compound but can be estimated from in vivo and/or in vitro data. Dosages necessary to achieve the MEC will depend on individual characteristics and route of administration. However, HPLC assays or bioassays can be used to determine plasma concentrations. Dosage intervals can also be determined using MEC value. Compositions should be administered using a regimen which maintains plasma levels above the MEC for 10-90% of the time, preferably between 30-90% and most preferably between 50-90%. In cases of local administration or selective uptake, the effective local concentration of the drug may not be related to plasma concentration.

It should be noted that the attending physician would know how to and when to terminate, interrupt or adjust administration due to toxicity or organ dysfunctions. Conversely, the attending physician would also know to adjust treatment to higher levels if the clinical response were not adequate (precluding toxicity). The magnitude of an administrated dose in the management of the disorder of interest will vary with the severity of the disease or condition to be treated and to the route of administration. The severity of the disease or condition may, for example, be evaluated, in part, by standard prognostic evaluation methods. Further, the dose and perhaps dose frequency, will also vary according to the age, body weight and response of the individual patient. A program comparable to that discussed above may be used in veterinary medicine.

Compounds, salts and compositions disclosed herein can be evaluated for efficacy and toxicity using known methods. For example, the toxicology of a particular compound, or of a subset of the compounds, sharing certain chemical moieties, may be established by determining in vitro toxicity towards a cell line, such as a mammalian, and preferably human, cell line. The results of such studies are often predictive of toxicity in animals, such as mammals, or more specifically, humans. Alternatively, the toxicity of particular compounds in an animal model, such as mice, rats, rabbits, dogs or monkeys, may be determined using known methods. The efficacy of a particular compound may be established using several recognized methods, such as in vitro methods, animal models, or human clinical trials. When selecting a model to determine efficacy, the skilled artisan can be guided by the state of the art to choose an appropriate model, dose, route of administration and/or regime.

### EXAMPLES

Additional embodiments are disclosed in further detail in the following examples, which are not in any way intended to limit the scope of the claims.

### Intermediate 1

### Methyl 7-bromo-6-chloro-3-(3-methoxy-3-oxopropyl)-1H-indole-2-carboxylate

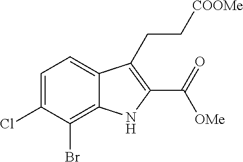 [SMILES: COC(=O)CCc1c(C(=O)OC)[nH]c2c(Br)c(Cl)ccc12]

To a stirred, 0° C. solution of 2-bromo-3-chloroaniline (25.0 g, 121 mmol) in conc. HCl (62.5 mL) and water (62.5 mL) was added a solution of NaNO₂ (8.79 g, 127 mmol) in water (30 mL). The ice bath was removed, and the reaction was stirred at rt for 1.5 h. A solution of KOAc (167 g, 1.70 mol) in water (250 mL) was added and the reaction was cooled to 0° C. Methyl 2-oxocyclopentane-1-carboxylate (17.29 g, 121.3 mmol) was added dropwise and the reaction was stirred at 0-5° C. for 30 min. The ice bath was removed, and the reaction was stirred at rt for 2 h. The solution was extracted with DCM (3×400 mL). The combined organic layers were washed with brine (200 mL), dried (Na₂SO₄), filtered and the solvent evaporated to afford methyl 1-((2-bromo-3-chlorophenyl)diazenyl)-2-oxocyclopentane-1-carboxylate (42 g, 96%) as a red solid. MS (LCMS) 361.1 [M+H]+.

To a stirred solution of methyl 1-((2-bromo-3-chlorophenyl)diazenyl)-2-oxocyclopentane-1-carboxylate (42.0 g, 117 mmol) in MeOH (420 mL) was added conc. H₂SO₄ (30.0 mL, 567 mol) at 0° C. The reaction was then stirred at 80° C. for 2 h. The reaction mixture was cooled to rt and the solids were filtered and washed with MeOH to afford dimethyl (E/Z)-2-(2-(2-bromo-3-chlorophenyl)hydrazineylidene)hexanedioate (28 g, 61%) as a yellow solid. MS (LCMS) 393.2 [M+H]⁺.

To a stirred solution of dimethyl (E/Z)-2-(2-(2-bromo-3-chlorophenyl)hydrazineylidene)-hexanedioate (29.0 g, 74.1 mmol) in MeOH (290 mL) was added conc. H₂SO₄ (50.0 mL, 938 mmol) at 0° C. The reaction was stirred at 80° C. for 4 days. The reaction was cooled to rt and the solid was filtered and washed with MeOH. The precipitate was dried under high vacuum to give Intermediate 1 (14 g, 50% yield) as an off-white solid. ¹H NMR (400 MHz, CDCl₃) δ 8.82 (br s, 1H), 7.61 (d, J=8.8 Hz, 1H), 7.23 (d, J=8.8 Hz, 1H), 3.98 (s, 3H), 3.63 (s, 3H), 3.37 (t, J=8.0 Hz, 2H), 2.68 (t, J=8.0 Hz, 2H); MS (LCMS) 375.9 [M+H]+.

### Intermediate 2

### Methyl 7-bromo-6-chloro-3-(3-methoxy-3-oxopropyl)-1-methyl-1H-indole-2-carboxylate

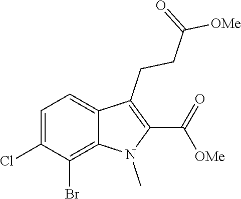 [SMILES: COC(=O)CCc1c(C(=O)OC)n(C)c2c(Br)c(Cl)ccc12]

To a stirred solution of Intermediate 1 (125 g, 373 mmol) in DMF (1.2 L) was added Cs₂CO₃ (65.3 g, 502 mmol) followed by MeI (95.14 g, 670.0 mmol) at 0° C. The reaction was stirred at rt for 3 h. After completion, the reaction was quenched with ice water (1 L) and allowed to stir for 30 min where a solid precipitated. The solid was filtered, washed with n-pentane and dried under high vacuum to afford Intermediate 2 (90 g, 70%) as a brown solid. MS (LCMS) 388.0 [M+H]⁺.

### Intermediate 3

### Methyl 7-bromo-6-chloro-3-(3-hydroxypropyl)-1-methyl-1H-indole-2-carboxylate

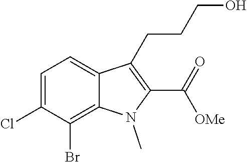 [SMILES: COC(=O)c1c(CCCO)c2ccc(Cl)c(Br)c2n1C]

To a stirred, 0° C. solution of Intermediate 2 (125 g, 322 mmol) in THF (1.2 L) was added 1 M BH₃THF in THF (1.77 L) over 30 min. The ice bath was removed, and the reaction was stirred at rt for 4 h. Upon completion by TLC, the reaction was cooled to 0° C. and quenched with methanol (1770 mL) and 6 N HCl (1770 mL). The mixture was extracted with EtOAc (2×1 L). The combined organic layers were washed with brine (1 L), dried (Na₂SO₄) and the solvent removed under reduced pressure to afford Intermediate 3 (130 g) as a brown solid. MS (LCMS) 362.0 [M+H]⁺.

### Intermediate 4

### Methyl 3-(3-acetoxypropyl)-7-bromo-6-chloro-1-methyl-1H-indole-2-carboxylate

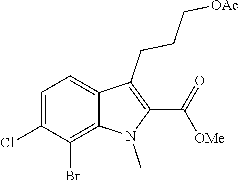 [SMILES: COC(=O)c1c(CCCOC(C)=O)c2ccc(Cl)c(Br)c2n1C]

To a stirred, 0° C. solution of Intermediate 3 (125 g, 322 mmol) in DCM (1.2 L) was added Et₃N (70.66 g, 698.0 mmol) and DMAP (3 g) followed by Ac₂O (53.4 g, 524). The ice bath was removed, and the reaction was stirred at rt for 1 h. Upon completion, the reaction was diluted with water (1 L) at 0° C. and extracted with DCM (2×1 L). The combined organic layers were washed with brine (1 L) and dried (Na₂SO₄). The solvent was evaporated, and the residue was purified by flash chromatography (SiO₂, EtOAc) to afford Intermediate 4 (96.3 g, 74%) as a white solid. ¹H NMR (400 MHz, CDCl₃) δ 7.50-7.47 (d, J=12.0 Hz, 1H), 7.24-7.21 (d, J=11.2 Hz, 1H), 4.32 (s, 3H), 4.08 (t, J=8.8 Hz 2H), 3.95 (s, 3H), 3.04 (t, J=10.4 Hz, 2H), 2.07 (s, 3H), 1.96 (m, 2H); MS (LCMS) 404.3 [M+H]⁺.

### Intermediate 5

### Ethyl (Z)-5-((tert-butyldiphenylsilyl)oxy)-2-hydroxy-4-oxopent-2-enoate

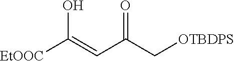

t-BuOK (3.60 kg, 32.1 mol) was added to THF (21 L) and the solution was cooled to 0° C. Diethyl oxalate (4.69 kg, 32.1 mol) was added slowly, maintaining the temperature below 0° C. The solution was stirred for 30 min at 0° C. 1-((tert-Butyldiphenylsilyl)oxy)propan-2-one (8.50 kg, 27.2 mol) was added slowly, maintaining the temperature below 0° C. The reaction mixture was stirred at 0° C. for 1 hr. Upon completion by TLC, the reaction was diluted with EtOAc (5 L). The resulting mixture was acidified with 1 N HCI to pH˜2 to 3. The phases were separated, and the aqueous phase was extracted with EtOAc (8 L, 3 L). The combined organic phases were washed with brine, dried (Na₂SO₄), filtered and concentrated to afford Intermediate 5 (12.4 kg, crude) as an oil.

### Intermediate 6

### Ethyl 5-(((tert-butyldiphenylsilyl)oxy)methyl)-1-methyl-1H-pyrazole-3-carboxylate

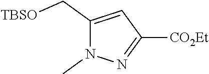 [SMILES: CCOC(=O)c1cc(CO[Si](C)(C)C(C)(C)C)n(C)n1]

Intermediate 5 (7.20 kg, 17.5 mol) was dissolved in 1,1,1,3,3,3-hexafluoropropan-2-ol (3.60 L) and trifluoroethanol (3.60 L). Two reactions of equal size were run simultaneously. The solution was cooled to 0° C. Methylhydrazine (2.01 kg, 17.5 mol) was added dropwise at 0° C. The ice bath was removed, and the mixture was stirred at rt for 2 h. Upon completion by TLC, the reactions were combined and concentrated. Water (7 L) was added and the mixture was extracted with EtOAc (5 L, 3 L, 2 L). The organic layers were washed with brine (3 L), dried (Na₂SO₄), filtered and the solvent removed. The residue was purified by flash chromatography (SiO₂, EtOAc/Pet. Ether) to afford Intermediate 6 (3.50 kg, 24%) as an oil. ¹H NMR (400 MHz, CDCl₃) δ 7.64-7.67 (m, 4H), 7.39-7.49 (m, 6H), 6.56 (s, 1H), 4.68 (s, 2H), 4.38-4.43 (m, 2H), 3.95 (s, 3H), 1.41 (t, J=7.0 Hz, 3H), 1.05 (s, 9H).

### Intermediate 7

### Ethyl 5-((acetylthio)methyl)-1-methyl-1H-pyrazole-3-carboxylate

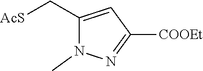

Intermediate 6 (3.50 kg, 8.28 mol) was dissolved in THF (7 L) at rt. 1 M TBAF (8.28 L) was added and the reaction was stirred at rt for 1 h. Upon completion by TLC, the solvent was removed under reduced pressure. Brine (10 L) was added to the residue. The mixture was extracted with EtOAc (10 L, 1 L×10). The combined organic phases were dried (Na₂SO₄), filtered and concentrated. The residue was purified by flash chromatography (SiO₂, EtOAc/Pet. ether) to afford ethyl 5-(hydroxymethyl)-1-methyl-1H-pyrazole-3-carboxylate (1.30 kg, 82%) as an oil.

A solution of compound ethyl 5-(hydroxymethyl)-1-methyl-1H-pyrazole-3-carboxylate (1.30 kg) in DCM (7.80 L) was cooled to 0° C. SOCl₂ (924 g, 7.76 mol) was added. The ice bath was removed, and the reaction was stirred at rt for 1 h. Upon completion by TLC, the mixture was concentrated to dryness. EtOAc (1.5 L) was added to the residue. The solution was washed with sat. NaHCO₃ (500 mL×2), dried (Na₂SO₄), filtered and concentrated to afford ethyl 5-(chloromethyl)-1-methyl-1H-pyrazole-3-carboxylate (1.28 kg, 89.5%) as an oil.

To a mixture of compound ethyl 5-(chloromethyl)-1-methyl-1H-pyrazole-3-carboxylate (1.28 kg, 6.32 mol) in CH₃CN (7.20 L) was added KI (1.05 kg, 6.32 mol) in one portion at rt under N₂. The mixture was stirred at rt for 15 min, then AcSK (1.08 kg, 9.48 mol) was added. The reaction was then stirred at 60° C. for 1 h where the reaction was determined to be complete by TLC. The mixture was concentrated to dryness. Water (5 L) and EtOAc (4 L) were added to the residue. The layers were separated. The organic phase was dried (Na2SO4) and concentrated. The residue was purified by flash chromatography (SiO₂, EtOAc) to afford Intermediate 7 (1.36 kg, 85%) as a brown solid. MS (LCMS) 243.0 [M+H]⁺.

### Intermediate 8

### 6-Bromo-8-((4-methoxybenzyl)oxy)quinoline

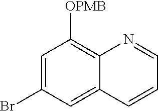

4-Bromo-2-methoxyaniline (2.40 kg, 11.9 mol) was added to a mixture of sodium 3-nitrobenzenesulfonate (4.01 kg, 17.8 mol) and propane-1,2,3-triol (5.14 kg, 55.8 mol) in H₂SO₄ (4.80 L) and H₂O (3.60 L) at rt under N₂. The reaction was stirred at 120° C. for 18 h. Upon completion by TLC, the reaction was cooled to rt and quenched slowly with 2 M NaOH to pH-10. The mixture was extracted with EtOAc (5 L×3). The combined organic phases were washed with brine (10 L), dried (Na₂SO₄), filtered and concentrated. The solid was dried under high vacuum to afford 6-bromo-8-methoxyquinoline (2.88 kg) as a brown oil. MS (LCMS) 238.0 [M+H]⁺.

6-Bromo-8-methoxyquinoline (2.88 kg, 12.1 mol) was added to 40% HBr (34.3 kg, 169 mol) at 25° C. under N₂. The reaction was stirred at 120° C. for 48 h where the reaction was determined to be complete by LCMS. The reaction was cooled to rt and quenched slowly with 4 M NaOH to pH-7. The mixture was extracted with EtOAc (7.5 L×2). The combined organic layers were washed with brine (5 L), dried (Na₂SO₄), filtered and concentrated. The crude material was triturated Pet. ether:EtOAc (˜5 L, 10:1) and dried under high vacuum to give 6-bromoquinolin-8-ol (1.80 kg, 62.7%) as light brown solid. MS (LCMS) 223.9 [M+H]⁺.

K₂CO₃ (2.22 kg, 16.1 mol) was added in portions to a mixture of PMB-Cl (1.51 kg, 9.64 mol) and 6-bromoquinolin-8-ol (1.80 kg, 8.03 mol) in DMF (10.8 L) under N₂ at rt. The reaction was stirred at rt for 12 h. Upon completion by TLC, the reaction was poured into water (20 L). A solid formed and the mixture was stirred for 15 min. The solid was collected by filtration and the filter cake was dissolved in DCM (10 L). The organic phase was washed with brine (5 L), dried (Na₂SO₄), filtered and concentrated. The residue was triturated with MTBE (5 L), filtered and dried under high vacuum to afford Intermediate 8 (1.70 kg, 60.0%) as a light brown solid. MS (LCMS) 344.0 [M+H]⁺.

### Intermediate 9

### Ethyl 5-(((8-((4-methoxybenzyl)oxy)quinolin-6-yl)thio)methyl)-1-methyl-1H-pyrazole-3-carboxylate

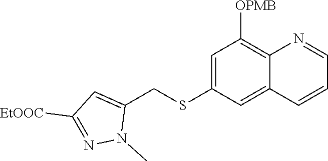

Pd₂(dba)₃ (19.9 g, 21.8 mmol) was added to a mixture of Intermediate 7 (151 g, 654 mmol), Intermediate 8 (150 g, 436 mmol), XPhos (19.9 g, 41.9 mmol), and K₂CO₃ (63.0 g, 457 mmol) in 1,4-dioxane (3 L) and H₂O (750 mL) under argon. The reaction was stirred at 100° C. for 12 h under argon. Nine reactions of equal scale were carried out simultaneously. Upon completion by TLC, the reactions were cooled to rt, combined and the solvent removed under reduced pressure. Water (2 L) and EtOAc (2 L) were added. The layers were separated, and the water layer was extracted with EtOAc (500 mL×3). The combined organic phases were dried (Na₂SO₄), filtered and concentrated. The crude product was purified by recrystallization with MTBE (5 L) to afford Intermediate 9 (1.30 kg, 64%) as a yellow solid. ¹H NMR (400 MHz, DMSO-d₆) δ 8.77-8.78 (m, 1H), 8.20-8.22 (m, 1H), 7.50-7.54 (m, 1H), 7.48-7.50 (m, 1H), 7.45-7.47 (m, 2H), 7.22 (s, 1H), 6.98 (d, J=8.4 Hz, 2H), 6.58 (s, 1H), 5.20 (s, 2H), 4.51 (s, 2H), 4.15-4.20 (m, 2H), 3.92 (s, 3H), 3.77 (s, 3H), 1.21 (t, J=7.2 Hz, 3H); MS (LCMS) 464.2 [M+H]⁺.

### Intermediate 10

### 6-(((3-(Chloromethyl)-1-methyl-1H-pyrazol-5-yl)methyl)thio)-8-((4-ethoxybenzyl)oxy)quinoline

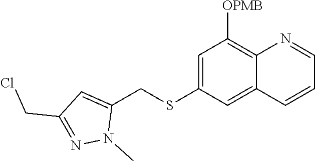

A solution of Intermediate 9 (1.30 kg, 2.80 mol) and CaCl₂ (623 g, 5.61 mol) in EtOH (6.5 L) and THF (1.3 L) was cooled to 0° C. NaBH₄ (318 g, 8.41 mol) was added at 0-10° C. The reaction was stirred at 50° C. for 3 h. Upon completion by TLC, the heating bath was removed, and the reaction was cooled in an ice bath. Sat. NH₄Cl (6.5 L) was added to the mixture slowly at 0-20° C. The mixture was extracted with EtOAc (2.5 L×3). The combined organic layers were dried (Na₂SO₄), filtered and concentrated. The crude residue was triturated EtOH:H₂O (1:1, 10L) and filtered. The filter cake was dried under nitrogen to afford (5-(((8-((4-methoxybenzyl)oxy)quinolin-6-yl)thio)methyl)-1-methyl-1H-pyrazol-3-yl)methanol (990 g) as light brown solid.

A mixture of (5-(((8-((4-methoxybenzyl)oxy)quinolin-6-yl)thio)methyl)-1-methyl-1H-pyrazol-3-yl)methanol (990 g, 2.35 mol), 2,6-lutidine (1.01 kg, 9.39 mol) and LiCl (498 g, 11.7 mol) in DMF (4.50 L) was cooled to 0° C. MsCl (543 g, 4.74 mol) was added dropwise at 0-10° C. The ice bath was removed, and the reaction was stirred at rt for 2 h. Upon completion by TLC, water (5 L) and EtOAc (3 L) were added. The layers were separated, and the water layer was extracted with EtOAc (2×500 mL). The combined organic phases were washed with brine (1 L×3), dried (Na₂SO₄), filtered and concentrated. The residue was purified by flash chromatography (SiO₂, EtOAc/Pet. ether) to afford Intermediate 10 (0.79 kg, 74.2%) as a light brown solid.

### Intermediate 11

### S-((5-(((8-((4-Methoxybenzyl)oxy)quinolin-6-yl)thio)methyl)-1-methyl-1H-pyrazol-3-yl)methyl) ethanethioate

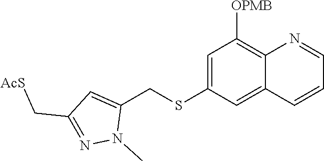

KI (298 g, 1.80 mol) and AcSK (410 g, 3.59 mol) were added to a solution of Intermediate 10 (790 g, 1.80 mol) in CH₃CN (4.7 L) at rt. The reaction was stirred at rt for 6 h. Upon completion by TLC, water (5 L) and EtOAc (3 L) were added. The layers were separated, and the water layer was extracted with EtOAc (500 mL). The combined organic phases were washed with brine (1 L×3), dried (Na₂SO₄), filtered and concentrated. The residue was purified by flash chromatography (SiO₂, EtOAc/Pet. ether) to afford Intermediate 11 (502 g, 56%) as a yellow solid. ¹H NMR (400 MHz, MeOH-d₄) δ 8.81-8.82 (m, 1H), 8.23-8.25 (m, 1H), 7.57-7.61 (m, 1H), 7.54 (d, J=8.0 Hz, 2H), 7.44 (d, 1H), 7.17 (d, 1H), 7.00 (d, J=8.4 Hz, 2H), 5.80 (s, 1H), 5.34 (s, 2H), 4.24 (s, 2H), 3.98 (s, 2H), 3.85 (s, 3H), 3.81 (s, 3H), 2.25 (s, 3H); MS (LCMS) 480.2 [M+H]⁺.

### Intermediate 12

### Methyl 5,6-dihydro-4H-pyrrolo[1,2-b]pyrazole-2-carboxylate

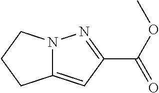

To a stirred solution of 5,6-dihydro-4H-pyrrolo[1,2-b]pyrazole-2-carboxylic acid (10.0 g, 65.7 mmol) in MeOH (100 mL) was added SOCl₂ (15.64 g, 131.4 mmol) at 0° C. The reaction was stirred at reflux for 6 h. Upon completion by TLC, the solvent was evaporated and co-distilled with MeOH. The solid was dried under high vacuum to afford Intermediate 12 (10 g, 92%) as an off-white solid. MS (LCMS) 167.1 [M+H]⁺.

### Intermediate 13

### (5,6-Dihydro-4H-pyrrolo[1,2-b]pyrazol-2-yl)methanol

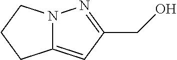

To a stirred solution of Intermediate 12 (10.0 g, 60.2 mmol) in THF (100 ml) was added 2M LiAlH₄ in THF (60.2 mL, 120 mmol) at 0° C. The reaction was stirred at rt for 2 h. Upon completion by TLC, the reaction was quenched with sat. NH₄Cl (100 mL) and extracted EtOAc (4×200 mL). The organic layers were combined, dried (Na₂SO₄), and filtered. The solvent was evaporated, and the residue was purified by flash chromatography (SiO₂, 20% EtOAc/Pet. ether) to afford Intermediate 13 (6.5 g, 78%) as an off-white solid. MS (LCMS) 139.1 [M+H]⁺.

### Intermediate 14

### (3-Bromo-5,6-dihydro-4H-pyrrolo[1,2-b]pyrazol-2-yl)methanol

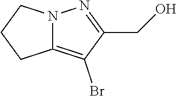 [SMILES: OCc1nn2c(c1Br)CCC2]

To a stirred solution of Intermediate 13 (4.00 g, 28.9 mmol) in DCM (50 mL) was added NBS (5.18 g, 28.9 mmol) at 0° C. The reaction was stirred at rt for 2 h. Upon completion by TLC, the reaction mixture was diluted with sat. NaHCO₃ (50 mL) and extracted with DCM (3×50 mL). The organic layers were combined, dried (Na₂SO₄), and filtered. The solvent was evaporated and the residue was triturated with pentane:ether (1:1) (3×20 mL) to provide Intermediate 14 (5.0 g, 79%) as a yellow solid. MS (LCMS) 217.0 [M+H]⁺.

### Intermediate 15

### 3-Bromo-2-(((4-methoxybenzyl)oxy)methyl)-5,6-dihydro-4H-pyrrolo[1,2-b]pyrazole

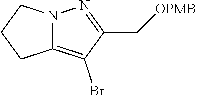

To a stirred solution of Intermediate 14 (4.00 g, 18.4 mmol) in DMF (40 mL) was added NaH (60%) (1.1 g, 27.64 mmol) at 0° C. The reaction was stirred at rt for 30 min. 1-(Chloromethyl)-4-methoxybenzene (4.04 g, 25.8 mmol) and KI (300 mg, 1.81 mmol) were added. The reaction was stirred at rt for 18 h. Upon completion, the reaction was quenched with sat. NH₄Cl (50 ml). The mixture was extracted with EtOAc (4×50 mL). The combined organic layers were washed with water (2×50 mL), brine (50 mL), and dried (Na₂SO₄). The solvent was evaporated, and the residue was purified by flash chromatography (SiO₂, 20% EtOAc/Pet. ether) to afford Intermediate 15 (3.4 g, 54%) as an off-white solid. MS (LCMS) 336.9 [M+H]⁺.

### Intermediate 16

### 2-(((4-Methoxybenzyl)oxy)methyl)-3-(4,4,5,5-tetramethyl-1,3,2-dioxaborolan-2-yl)-5,6-dihydro-4H-pyrrolo[1,2-b]pyrazole

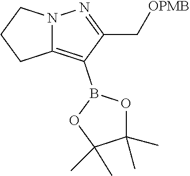

To a stirred solution of Intermediate 15 (10.0 g, 29.8 mmol) in THF (200 mL) was added 1.6 M n-BuLi in hexanes (27.9 mL, 44.6 mmol) at −78° C. The reaction was stirred at −78° C. for 50 min. 2-Isopropoxy-4,4,5,5-tetramethyl-1,3,2-dioxaborolane (22.14 mL, 119.04 mmol) was added at −78° C. and the reaction was stirred at −78° C. for 1 h. Upon completion, the reaction temperature was gradually increased to rt. The solvent was removed by evaporation under reduced pressure and the reaction was diluted with EtOAc (200 mL). The mixture was filtered through a Celite pad and washed with EtOAC (2×50 mL). The solvent was evaporated, and the residue was purified by flash chromatography (SiO₂, 30-50% EtOAc/Pet. ether) to afford Intermediate 16 (8.8 g, 88%) as a white solid. MS (LCMS) 385.4 [M+H]⁺.

### Intermediate 17

### Methyl 3-(3-acetoxypropyl)-6-chloro-7-(2-(((4-methoxybenzyl)oxy)methyl)-5,6-dihydro-4H-pyrrolo[1,2-b]pyrazol-3-yl)-1-methyl-1H-indole-2-carboxylate

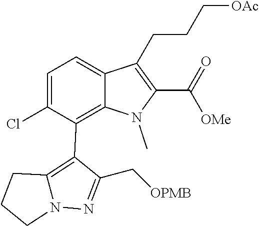

To a stirred solution of Intermediate 16 (11.4 g, 30.0 mmol) in 1,4-dioxane (110 mL) were added Intermediate 4 (6.03 g, 15.0 mmol) and Cs₂CO₃ (19.5 g, 60.0 mmol). The resulting solution was degassed with Argon for 10 min. Pd(dtbpf)Cl₂ (1.17 g, 1.80 mmol) was added and the reaction was degassed for 10 min. The reaction was heated at 100° C. for 16 h. Upon completion, the reaction was cooled to rt and the solvent was evaporated under reduced pressure. The residue was diluted with EtOAc (150 mL) and passed through Celite pad and washed with EtOAc (50 mL). The solvent was evaporated, and the residue was purified by flash chromatography (SiO₂, 50-70% EtOAc/Pet. ether) to afford Intermediate 17 (4.2 g, 30%) as a yellow oil. MS (LCMS) 580.4 [M+H]⁺

### Intermediate 18

### Methyl 3-(3-acetoxypropyl)-6-chloro-7-(2-(hydroxymethyl)-5,6-dihydro-4H-pyrrolo[1,2-b]pyrazol-3-yl)-1-methyl-1H-indole-2-carboxylate

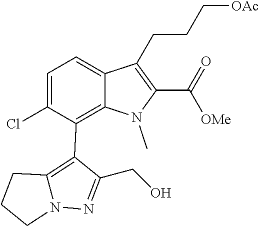 [SMILES: COC(=O)c1c(CCCOC(C)=O)c2ccc(Cl)c(-c3c(CO)nn4c3CCC4)c2n1C]

To a stirred, 0° C. solution of Intermediate 17 (9.0 g, 15.5 mmol) in DCM (90 mL) was added TFA (17.6 mL, 155 mmol). The ice bath was removed, and the reaction was stirred at rt for 1.5 h. The reaction was quenched with sat. NaHCO₃ (100 mL) at 0° C. The solid was recovered by filtration and washed with water (100 mL). The solid was dissolved in DCM (500 mL) and dried (Na₂SO₄). The solvent was evaporated, and the residue was purified by flash chromatography (SiO₂, 70% EtOAc/Pet. ether) to afford Intermediate 18 (4.9 g, 68%) as an oil. MS (LCMS) 460.2 [M+H]⁺.

### Intermediate 19

### Methyl 3-(3-acetoxypropyl)-6-chloro-7-(2-(chloromethyl)-5,6-dihydro-4H-pyrrolo[1,2-b]pyrazol-3-yl)-1-methyl-1H-indole-2-carboxylate

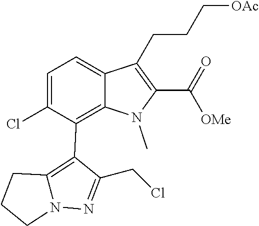 [SMILES: COC(=O)c1c(CCCOC(C)=O)c2ccc(Cl)c(-c3c(CCl)nn4c3CCC4)c2n1C]

To a stirred solution of Intermediate 18 (7.00 g, 15.2 mmol) in DCM (70 mL) under argon was added SOCl₂ (1.32 mL, 18.3 mmol) at 0° C. The reaction was stirred at rt for 1 h. The reaction was concentrated and partitioned between DCM (250 mL) and sat. NaHCO₃ (100 mL). The organic layer was separated, dried (Na₂SO₄), and the solvent evaporated to afford Intermediate 19 (7.0 g, 96%) as a semi solid. MS (LCMS) 478.3 [M+H]⁺.

### Intermediate 20

### Methyl 3-(3-acetoxypropyl)-6-chloro-7-(2-(iodomethyl)-5,6-dihydro-4H-pyrrolo[1,2-b]pyrazol-3-yl)-1-methyl-1H-indole-2-carboxylate

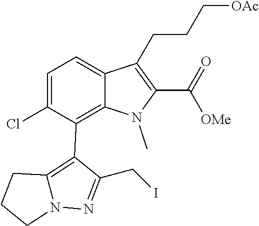 [SMILES: COC(=O)c1c(CCCOC(C)=O)c2ccc(Cl)c(-c3c(CI)nn4c3CCC4)c2n1C]

To a stirred solution of Intermediate 19 (7.00 g, 14.7 mmol) in dry MeCN (70 mL) was added NaI (3.93 g, 26.4 mmol) at rt. The reaction was heated to 80° C. for 1 h. Upon completion, the solvent was evaporated, and the mixture was diluted with water (250 mL). The mixture was extracted with EtOAc (3×200 mL). The combined organic layers were dried (Na₂SO₄), filtered and evaporated to afford Intermediate 20 (8 g) as a semi solid. MS (LCMS) 570.3 [M+H]⁺.

### Intermediate 21

### Methyl 6-chloro-3-(3-hydroxypropyl)-7-(2-((((5-(((8-((4-methoxybenzyl)oxy)quinolin-6-yl)thio)methyl)-1-methyl-1H-pyrazol-3-yl)methyl)thio)methyl)-5,6-dihydro-4H-pyrrolo [1,2-b]pyrazol-3-yl)-1-methyl-1H-indole-2-carboxylate

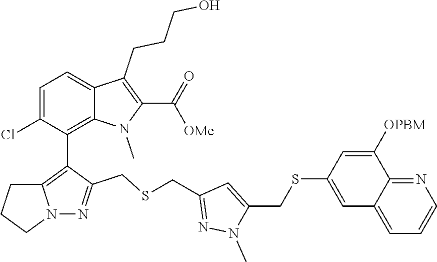

To a stirred solution of Intermediate 20 (4.00 g, 7.01 mmol) in MeOH (40 mL) and THF (10 mL) was added K₂CO₃ (0.968 g, 7.02 mmol). The mixture was degassed with Argon for 10 min. In another flask, Intermediate 11 (3.30 g, 7.02 mmol) in methanol (15 mL) was degassed with Argon for 10 min and then added to the reaction mixture dropwise over 30 min. The reaction was stirred at rt for 16 h. The solvent was evaporated, and the reaction mixture was diluted with water (150 mL). The mixture was extracted with EtOAc (3×500 mL). The combined organic layers were dried (Na₂SO₄), filtered and the solvent was evaporated. The residue was purified by flash chromatography (SiO₂, 100% EtOAc/) to afford Intermediate 21 (6.2 g, 45% 2 steps) as a solid. MS (LCMS) 837.5 [M+H]⁺.

### Intermediate 22

### Methyl 6-chloro-3-(3-hydroxypropyl)-7-(2-((((5-(((8-hydroxyquinolin-6-yl)thio)methyl)-1-methyl-1H-pyrazol-3-yl)methyl)thio)methyl)-5,6-dihydro-4H-pyrrolo[1,2-b]pyrazol-3-yl)-1-methyl-1H-indole-2-carboxylate

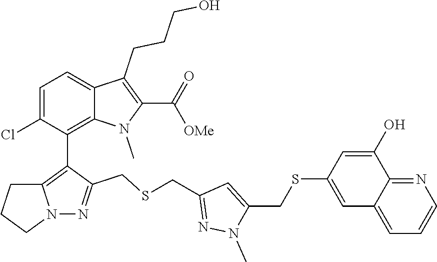 [SMILES: COC(=O)c1c(CCCO)c2ccc(Cl)c(-c3c(CSCc4cc(CSc5cc(O)c6ncccc6c5)n(C)n4)nn4c3CCC4)c2n1C]

To a stirred solution of Intermediate 21 (6.20 g, 7.40 mmol) in DCM (90 mL) was added TFA (5.70 mL, 74.0 mmol) at 0° C. The reaction was stirred at rt for 1.5 h. The reaction was concentrated and partitioned between DCM (200 mL) and sat. NaHCO₃ (200 mL). The organic layer was separated, dried (Na₂SO₄), filtered and the solvent was evaporated. The residue was purified by flash chromatography (SiO₂, 100% EtOAc) to afford Intermediate 22 (3.2 g, 60%) as a solid. MS (LCMS) 717.6 [M+H]⁺.

### Intermediate 23

### Methyl (Z)-1⁶-chloro-1¹,6¹-dimethyl-2⁵,26-dihydro-1¹H,2⁴H,6¹H-10-oxa-4,8-dithia-9(6,8)-quinolina-1(7,3)-indola-2(3,2)-pyrrolo[1,2-b]pyrazola-6(3,5)-pyrazolacyclotridecaphane-1²-carboxylate

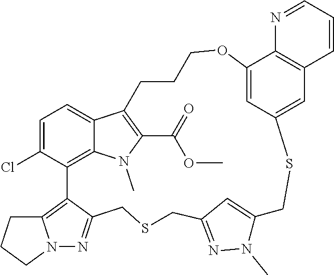 [SMILES: COC(=O)c1c2c3ccc(Cl)c(c3n1C)-c1c(nn3c1CCC3)CSCc1cc(n(C)n1)CSc1cc(c3ncccc3c1)OCCC2]

To a stirred solution of TPP (730 mg, 2.79 mmol) in toluene (5 mL) was added a solution of di-tert-butyl diazo-1,2-dicarboxylate (641 mg, 2.79 mmol) and Intermediate 22 (1.00 g, 1.39 mmol) in THF (5 mL). The reaction was stirred at 90° C. for 1 h. The reaction was diluted with EtOAc (50 mL) and washed with water (50 mL), sat. NaHCO₃ (50 mL) and brine (50mL). The organic layer was dried (Na₂SO₄), filtered and the solvent evaporated. The residue was purified by flash chromatography (SiO₂, 100% EtOAc) to afford Intermediate 23 (650 mg, 70%) as an off-white solid. MS (LCMS) 699.5 [M+H]⁺.

### Intermediate 24A

### (Rₐ)-(+)-Methyl (Z)-1⁶-chloro-1¹,6¹-dimethyl-2⁵,2⁶,9¹,9²,9³,9⁴-hexahydro-1¹H,2⁴H,6¹H-10-oxa-4,8-dithia-9(6,8)-quinolina-1(7,3)-indola-2(3,2)-pyrrolo[1,2-b]pyrazola-6(3,5)-pyrazolacyclotridecaphane-1²-carboxylate

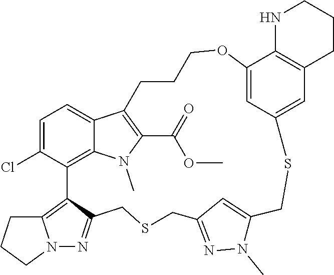

### Intermediate 24B

### (Sₐ)-(−)-Methyl (Z)-1⁶-chloro-1¹,6¹-dimethyl-2⁵,2⁶,9¹,9²,9³,9⁴-hexahydro-1¹H,2⁴H,6¹H-10-oxa-4,8-dithia-9(6,8)-quinolina-1(7,3)-indola-2(3,2)-pyrrolo[1,2-b]pyrazola-6(3,5)-pyrazolacyclotridecaphane-1²-carboxylate

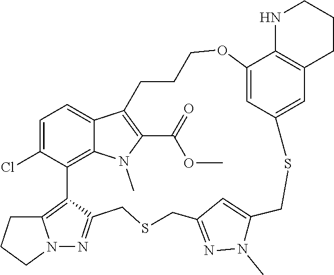

To a stirred solution of Intermediate 23 (600 mg, 0.858 mmol) in MeOH (6 mL) and AcOH (6 mL) was added NaCNBH₃ (532 mg, 8.58 mmol) at rt. The reaction was stirred at 70° C. for 2 h. Upon completion, the reaction was concentrated and partitioned between DCM (50 mL) and sat. NaHCO₃ (20 mL). The organic layer was separated, dried (Na₂SO₄), filtered and the solvent evaporated. The residue was purified by flash chromatography (SiO₂, 100% EtOAc) to afford racemic methyl (Z)-1⁶-chloro-1¹,6¹-dimethyl-2⁵,2⁶,9¹,9²,9³,9⁴-hexahydro-1¹H,2⁴H,6¹H-10-oxa-4,8-dithia-9(6,8)-quinolina-1(7,3)-indola-2(3 ,2)-pyrrolo [1,2-b]pyrazola-6(3,5)pyrazolacyclo-tridecaphane-1²-carboxylate (150 mg, 24%) as an off-white solid. MS (LCMS) 703.3 [M+H]⁺. The atropisomers were separated chiral SFC chromatography (Chiralcel OJ-3 (30×250 mm) column, 30% MeOH) to give peak 1 (Intermediate 24A, 54 mg) and peak 2 (Intermediate 24B, 54 mg). Intermediate 24A: off-white solid; 99.9% chiral purity; MS (LCMS) 703.7 [M+H]⁺. Intermediate 24B: off-white solid; 99.3% chiral purity; MS (LCMS) 703.9 [M+H]⁺. The absolute stereochemistry of Intermediate 24A and Intermediate 24B was arbitrarily assigned.

### Example 1A

### (Rₐ)-(+)-(Z)-1⁶-Chloro-1¹,6¹-dimethyl-2⁵,2⁶,9¹,9²,9³,9⁴-hexahydro-1¹H,2⁴H,6¹H-10-oxa-4,8-dithia-9(6,8)-quinolina-1(7,3)-indola-2(3,2)-pyrrolo[1,2-b]pyrazola-6(3,5)-pyrazolacyclotridecaphane-1²-carboxylic acid

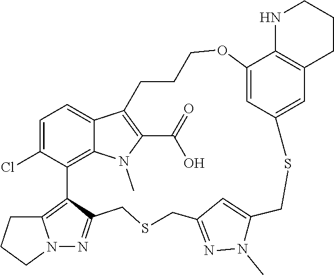

To a stirred solution of Intermediate 24A (40 mg, 0.057 mmol) in MeOH/THF/H₂O (1:1:1, 2.5 mL) was added LiOH·H₂O (36 mg, 0.85 mmol) at rt. The reaction was stirred at 70° C. for 3 h. Upon completion, the solvent was evaporated. The aqueous layer was acidified to pH 2 using 2 N aq. HCl. The solid was filtered and washed with water (5 mL). The solid was collected and dried under vacuum to afford Example 1A (25 mg, 64%) as an off-white solid. 99.4% chiral purity; NMR (400 MHz, DMSO-d₆) δ 13.20 (brs, 1H), 7.74 (d, J=8.8 Hz, 1H), 7.12 (d, J=8.4 Hz, 1H), 6.58 (s, 1H), 6.11 (s, 1H), 5.20 (s, 1H), 4.79 (s, 1H), 4.13-4.09 (m, 2H), 3.94 (d, J=15.2 Hz, 1H), 3.82 (d, J=14.8 Hz, 1H), 3.59-3.18 (m, 8H), 3.49-3.48 (m, 1H) 3.30-3.20 (m, 5H), 3.11-3.09 (m, 1H), 2.70-2.50 (m, 7H), 2.20 (brs, 1H), 1.98 (brs, 1H), 1.78-1.75 (m, 2H); MS (LCMS) 689.3 [M+H]⁺.

### Example 1B

### (Sₐ)-(−)-(Z)-1⁶-Chloro-1¹,6¹-dimethyl-2⁵,2⁶,9¹,9²,9³,9⁴-hexahydro-1¹H,2⁴H,6¹H-10-oxa-4,8-dithia-9(6,8)-quinolina-1(7,3)-indola-2(3 ,2)-pyrrolo[1,2-b]pyrazola-6(3,5)-pyrazolacyclotridecaphane-1²-carboxylic acid

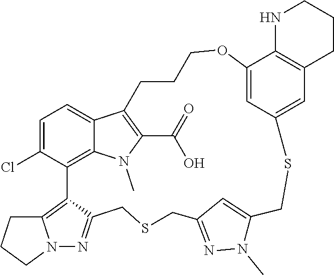

To a stirred solution of Intermediate 24B (50 mg, 0.071 mmol) in MeOH/THF/H₂O (1:1:1, 2.5 mL) was added LiOH·H₂O (44 mg, 1.07 mmol) at rt. The reaction was stirred at 70° C. for 3 h. Upon completion, the solvent was evaporated. The aqueous layer was acidified to pH 2 using 2 N aq. HCl. The solid was filtered and washed with water (5 mL). The solid was collected and dried under vacuum to afford Example 1B (35 mg, 72%) as an off-white solid. 99.8% chiral purity; ¹H NMR (400 MHz, DMSO-d₆) δ 13.20 (brs, 1H), 7.71 (d, J=8.8 Hz, 1H), 7.09 (d, J=8.4 Hz, 1H), 6.55 (s, 1H), 6.05 (s, 1H), 5.20 (s, 1H), 4.79 (s, 1H), 4.13-4.10 (m, 3H), 3.76 (d, J=14.8 Hz, 1H), 3.70-3.60 (m, 5H), 3.50 (s, 3H), 3.44-3.40 (m, 1H), 3.30-3.20 (m, 4H), 3.00 (d, J=16 Hz, 1H), 2.70-2.50 (m, 8H), 2.10-2.10 (m, 2H), 1.78-1.75 (m, 2H); MS (LCMS) 689.3 [M+H]⁺. The absolute stereochemistry of Example 1A and Example 1B was arbitrarily assigned.

### Intermediate 25

### Methyl 4,5,6,7-tetrahydropyrazolo[1,5-a]pyridine-2-carboxylate

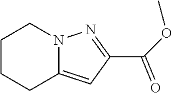

Intermediate 25 was synthesized from 4,5,6,7-tetrahydropyrazolo[1,5-a]pyridine-2-carboxylic acid following a procedure from the preparation of Intermediate 12. MS (LCMS) 181.1 [M+H]⁺.

### Intermediate 26

### (4,5,6,7-Tetrahydropyrazolo[1,5-a]pyridin-2-yl)methanol

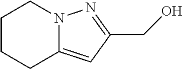 [SMILES: OCc1cc2n(n1)CCCC2]

Intermediate 26 was synthesized from Intermediate 25 following a procedure for the preparation of Intermediate 13. MS (LCMS) 153.1 [M+H]⁺.

### Intermediate 27

### (3-Bromo-4,5,6,7-tetrahydropyrazolo[1,5-a]pyridin-2-yl)methanol

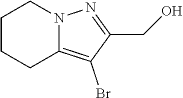 [SMILES: OCc1nn2c(c1Br)CCCC2]

Intermediate 27 was synthesized from Intermediate 26 following a procedure for the preparation of Intermediate 14. MS (LCMS) 231.0 [M+H]⁺.

### Intermediate 28

### 3-Bromo-2-(((4-methoxybenzyl)oxy)methyl)-4,5,6,7-tetrahydropyrazolo[1,5-a]pyridine

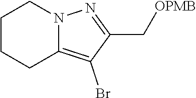

Intermediate 28 was synthesized from Intermediate 27 following a procedure for the preparation of the Intermediate 15. MS (LCMS) 351.0 [M+H]⁺.

### Intermediate 29

### 2-(((4-Methoxybenzyl)oxy)methyl)-3-(4,4,5,5-tetramethyl-1,3,2-dioxaborolan-2-yl)-4,5,6,7-tetrahydropyrazolo[1,5-a]pyridine

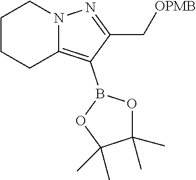

Intermediate 29 was synthesized from Intermediate 28 following a procedure for the preparation of Intermediate 16. MS (LCMS) 399.4 [M+H]⁺.

### Intermediate 30

### Methyl 3-(3-acetoxypropyl)-6-chloro-7-(2-(((4-methoxybenzyl)oxy)methyl)-4,5,6,7-tetrahydropyrazolo[1,5-a]pyridin-3-yl)-1-methyl-1H-indole-2-carboxylate

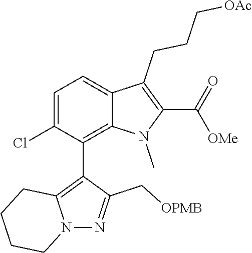

Intermediate 30 was synthesized from Intermediate 29 and Intermediate 4 following a procedure from the preparation of Intermediate 17. MS (LCMS) 594.4 [M+H]⁺.

### Intermediate 31

### Methyl 3-(3-acetoxypropyl)-6-chloro-7-(2-(hydroxymethyl)-4,5,6,7-tetrahydropyrazolo[1,5-a]pyridin-3-yl)-1-methyl-1H-indole-2-carboxylate

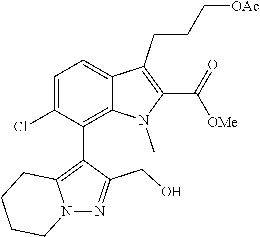 [SMILES: COC(=O)c1c(CCCOC(C)=O)c2ccc(Cl)c(-c3c(CO)nn4c3CCCC4)c2n1C]

Intermediate 31 was synthesized from Intermediate 30 following a procedure for the preparation of Intermediate 18. MS (LCMS) 474.4 [M+H]⁺.

### Intermediate 32

### Methyl 3-(3-acetoxypropyl)-6-chloro-7-(2-(chloromethyl)-4,5,6,7-tetrahydropyrazolo[1,5-a]pyridin-3-yl)-1-methyl-1H-indole-2-carboxylate

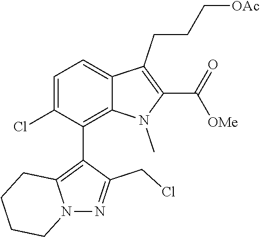 [SMILES: COC(=O)c1c(CCCOC(C)=O)c2ccc(Cl)c(-c3c(CCl)nn4c3CCCC4)c2n1C]

Intermediate 32 was synthesized from Intermediate 31 following a procedure for the preparation of Intermediate 19. MS (LCMS) 492.4 [M+H]⁺.

### Intermediate 33

### Methyl 3-(3-acetoxypropyl)-6-chloro-7-(2-(iodomethyl)-4,5,6,7-tetrahydropyrazolo[1,5-a]pyridin-3-yl)-1-methyl-1H-indole-2-carboxylate

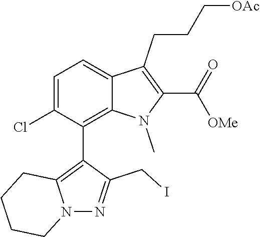 [SMILES: COC(=O)c1c(CCCOC(C)=O)c2ccc(Cl)c(-c3c(CI)nn4c3CCCC4)c2n1C]

Intermediate 33 was synthesized from Intermediate 32 following a procedure for the preparation of Intermediate 20. MS (LCMS) 584.2 [M+H]⁺.

### Intermediate 34

### Methyl 6-chloro-3-(3-hydroxypropyl)-7-(2-((((5-(((8-((4-methoxybenzyl)oxy)quinolin-6-yl)thio)methyl)-1-methyl-1H-pyrazol-3-yl)methyl)thio)methyl)-4,5,6,7-tetrahydropyrazolo[1,5-a]pyridin-3-yl)-1-methyl-1H-indole-2-carboxylate

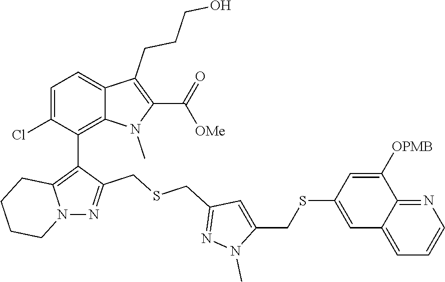

Intermediate 34 was synthesized from Intermediate 33 and Intermediate 11 following a procedure for the preparation of Intermediate 21. MS (LCMS) 851.5 [M+H]⁺.

### Intermediate 35

### Methyl 6-chloro-3-(3-hydroxypropyl)-7-(2-((((5-(((8-hydroxyquinolin-6-yl)thio)methyl)-1-methyl-1H-pyrazol-3-yl)methyl)thio)methyl)-4,5,6,7-tetrahydropyrazolo[1,5-a]pyridin-3-yl)-1-methyl-1H-indole-2-carboxylate

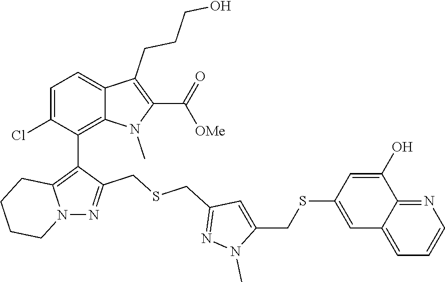 [SMILES: COC(=O)c1c(CCCO)c2ccc(Cl)c(-c3c(CSCc4cc(CSc5cc(O)c6ncccc6c5)n(C)n4)nn4c3CCCC4)c2n1C]

Intermediate 35 was synthesized from Intermediate 34 following a procedure for the preparation of Intermediate 22. MS (LCMS) 731.5 [M+H]⁺

### Intermediate 36

### Methyl (Z)-1⁶-chloro-1¹,6¹-dimethyl-2⁴,2⁵,2⁶,2⁷-tetrahydro-1¹H,6¹H-10-oxa-4,8-dithia-9(6,8)-quinolina-2(3 ,2)-pyrazolo[1,5-a]pyridina-1(7,3)-indola-6(3 ,5)-pyrazolacyclotridecaphane carboxylate

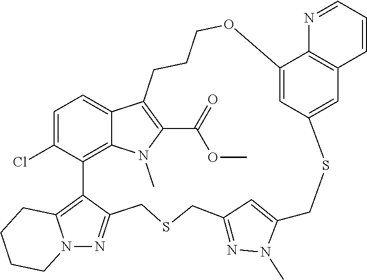 [SMILES: COC(=O)c1c2c3ccc(Cl)c(c3n1C)-c1c(nn3c1CCCC3)CSCc1cc(n(C)n1)CSc1cc(c3ncccc3c1)OCCC2]

Intermediate 36 was synthesized from Intermediate 35 following a procedure for the preparation of Intermediate 23. MS (LCMS) 712.9 [M+H]⁺.

### Intermediate 37A

### (Rₐ)-(+)-Methyl (Z)-1⁶-chloro-1¹,6¹-dimethyl-2⁴,2⁵,2⁶,2⁷,9¹,9²,9³,9⁴-octahydro-1¹H,6¹H-10-oxa-4,8-dithia-9(6,8)-quinolina-2(3,2)-pyrazolo[1,5-a]pyridina-1(7,3)-indola-6(3,5)-pyrazolacyclotridecaphane-1²-carboxylate

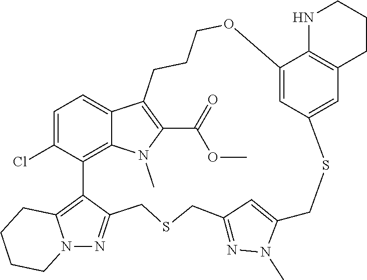 [SMILES: COC(=O)c1c2c3ccc(Cl)c(c3n1C)-c1c(nn3c1CCCC3)CSCc1cc(n(C)n1)CSc1cc3c(c(c1)OCCC2)NCCC3]

### Intermediate 37B

### (Sₐ)-(−)-Methyl (Z)-1⁶-chloro-1¹,6¹-dimethyl-2⁴,2⁵,2⁶,2⁷,9¹,9²,9³,9⁴-octahydro-1¹H,6¹H-10-oxa-4,8-dithia-9(6,8)-quinolina-2(3,2)-pyrazolo[1,5-a]pyridina-1(7,3)-indola-6(3,5)-pyrazolacyclotridecaphane-1²-carboxylate

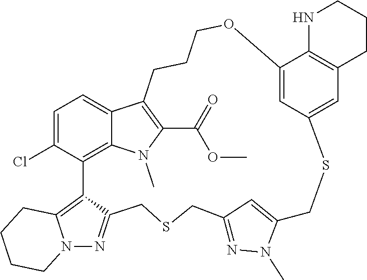

Intermediates 37A and 37B were synthesized from Intermediate 36 following a procedure for the preparation Intermediates 24A and 24B to give racemic methyl (Z)-1⁶-chloro-1¹,6¹-dimethyl-2⁴,2⁵,2⁶,2⁷,9¹,9²,9³,9⁴-octahydro-1¹H,6¹H-10-oxa-4,8-dithia-9(6,8)-quinolina-2(3,2)-pyrazolo[1,5-a]pyridina-1(7,3)-indola-6(3,5)-pyrazolacyclotridecaphane-1²-carboxylate (700 mg). The atropisomers were separated by chiral SFC chromatography (Chiralcel-OJ-H (30×250 mm) column, 30% MeOH) to give peak 1 (Intermediate 37A, 300 mg) and peak 2 (Intermediate 37B, 310 mg). Intermediate 37A: off-white solid; 99.9% chiral purity; MS (LCMS) 717.5 [M+H]⁺. Intermediate 37B: off-white solid; 99.7% chiral purity; MS (LCMS) 717.5 [M+H]⁺. The absolute stereochemistry of Intermediate 37A and Intermediate 37B was arbitrarily assigned.

### Example 2A

### (Rₐ)-(+)-(Z)-1⁶-chloro-1¹,6¹-dimethyl-2⁴,2⁵,2⁶,2⁷,9¹,9²,9³,9⁴-octahydro-1¹H,6¹H-10-oxa-4,8- dithia-9(6,8)-quinolina-2(3 ,2)-pyrazolo[1,5-a]pyridina-1(7 ,3)-indola-6(3,5)-pyrazolacyclotridecaphane-1²-carboxylic acid

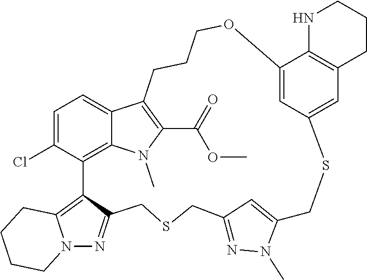

Example 2A was synthesized from Intermediate 37A following a procedure for the preparation of Example 1. Example 2A: (265 mg, 90%), yellow solid; 99.4% chiral purity; ¹H NMR (400 MHz, DMSO-d₆) δ 13.24 (s, 1H), 7.73 (d, J=8.8 Hz, 1H), 7.11 (d, J=8.4 Hz, 1H), 6.56 (s, 1H), 6.05 (s, 1H), 5.23 (bs, 1H), 4.75 (s, 1H), 4.10-4.05 (m, 2H), 3.91 (d, J=14.8 Hz, 1H), 3.77 (d, J=15.2 Hz 1H), 3.58-3.57 (m, 7H), 3.45-3.40 (m, 1H), 3.24-3.08 (m, 4H), 3.05-2.95 (m, 2H), 2.66-2.63 (m, 3H), 2.40-2.33 (m, 3H), 2.17-2.16 (m, 1H), 1.97 (s, 3H), 1.76 (bs, 4H); MS (LCMS) 703.4 [M+H]⁺.

### Example 2B

### (Sₐ)-(−)-(Z)-1⁶-Chloro-1¹,6¹-dimethyl-2⁴,2⁵,2⁶,2⁷,9¹,9²,9³,9⁴-octahydro-1¹H,6¹H-10-oxa-4,8- dithia-9(6,8)-quinolina-2(3,2)-pyrazolo[1,5-a]pyridina-1(7,3)-indola-6(3,5)-pyrazolacyclotridecaphane-1²-carboxylic acid

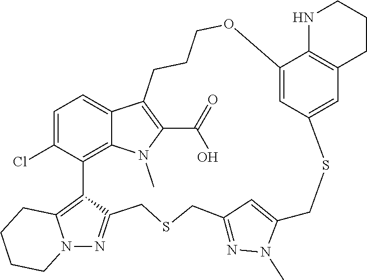

Example 2B was synthesized from Intermediate 37B following a procedure for the preparation of Example 1. Example 2B: (275 mg, 93%), yellow solid; 97.6% chiral purity; ¹H NMR (400 MHz, DMSO-d₆) δ 13.2 (s, 1H), 7.75 (d, J=8.4 Hz, 1H), 6.93 (d, J=8.8 Hz, 1H), 6.46 (s, 1H), 6.20 (s, 1H), 5.20 (s, 1H), 4.83 (s, 1H), 4.09-4.03 (m, 2H), 3.90 (d, J=15.2 Hz, 1H), 3.75 (d, J=15.2 Hz, 1H), 3.65-3.48 (m, 9H), 3.33-3.30 (m, 6H), 2.70-2.60 (m, 3H), 2.49-2.30 (m, 2H), 2.2-2.10 (m, 1H), 2.05-1.90 (m, 3H), 1.85-1.70 (m, 4H); MS (LCMS) 703.4 [M+H]+. The absolute stereochemistry of Example 2A and Example 2B was arbitrarily assigned.

### Intermediate 38

### Methyl 3-(3-acetoxypropyl)-6-chloro-7-(2-formyl-5,6-dihydro-4H-pyrrolo[1,2-b]pyrazol-3-yl)-1-methyl-1H-indole-2-carboxylate

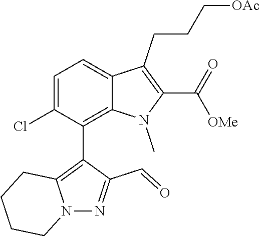 [SMILES: COC(=O)c1c(CCCOC(C)=O)c2ccc(Cl)c(-c3c(C=O)nn4c3CCCC4)c2n1C]

To a stirred solution of Intermediate 18 (6.60 g, 14.4 mmol) in CH₂Cl₂ (70 mL) was added Dess-Martin periodinane (6.70 g, 15.8 mmol) and NaHCO₃ (5.43 g, 64.7 mmol) at 0° C. The ice bath was removed, and the reaction was stirred at rt for 1 h. Upon completion, the reaction was diluted with DCM (200 mL). The mixture was washed with water (100 mL) and brine (100 mL). The organic layer was dried (Na₂SO₄), filtered and concentrated under reduced pressure. The residue was purified by flash chromatography (SiO₂, EtOAc/Pet. ether) to afford Intermediate 38 (4.5 g, 68%) as an off-white solid. MS (ESI) 458.3 [M+H]⁺.

### Intermediate 39

### Methyl 6-chloro-3-(3-hydroxypropyl)-1-methyl-7-(2-((methylamino)methyl)-5,6-dihydro-4H-pyrrolo[1,2-b]pyrazol-3-yl)-1H-indole-2-carboxylate

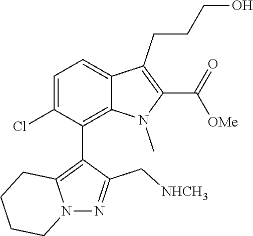 [SMILES: CNCc1nn2c(c1-c1c(Cl)ccc3c(CCCO)c(C(=O)OC)n(C)c13)CCCC2]

To a stirred solution of Intermediate 38 (3.50 g, 7.66 mmol) in MeOH (35 mL) was added TEA (2.12 mL, 15.3 mmol). The reaction was stirred at rt for 10 min and then cooled to 0° C. 2M methylamine in THF (7.65 mL, 15.3 mmol) was added at 0° C. The ice bath was removed, and the reaction was stirred at rt for 12 h. NaBH₄ (0.565 g, 14.9 mmol) was added portionwise at 0° C. The ice bath was removed, and the reaction was stirred at rt for 4 h. Upon completion by TLC, the reaction was evaporated to dryness and diluted with cold water (100 mL). The mixture was extracted with EtOAc (2×200 mL), washed with brine (100 mL), dried (Na₂SO₄), filtered and concentrated under reduced pressure. The residue was purified by flash chromatography (SiO₂, MeOH/DCM) to afford Intermediate 39 (1.8 g, 56%) as an off-white solid. MS (ESI) 431.8 [M+H]⁺.

### Intermediate 40

### Methyl 6-chloro-3-(3-hydroxypropyl)-7-(2-((((5-(((8-((4-methoxybenzyl)oxy)quinolin-6-yl)thio)methyl)-1-methyl-1H-pyrazol-3-yl)methyl)(methyl)amino)methyl)-5,6-dihydro-4H-pyrrolo[1,2-b]pyrazol-3-yl)-1-methyl-1H-indole-2-carboxylate

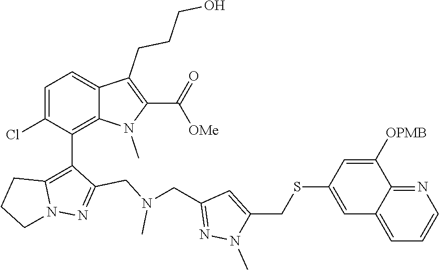

To a stirred solution of Intermediate 39 (900 mg, 2.09 mmol) in DMF (10 mL) was added K₂CO₃ (578 mg, 4.19 mmol) at 0° C. The reaction was stirred for 15 min and Intermediate 10 (1.19 g, 2.72 mmol) was added at 0° C. and then stirred for 16 h at rt. Two reactions of equal scale were run simultaneously. Upon completion by TLC, the reactions were quenched with cold water (50 mL) and extracted with EtOAc (100 mL). The combined organic layers were washed with brine (100 mL), dried (Na₂SO₄), filtered and concentrated under reduced pressure. The residue was purified by flash chromatography (SiO₂, MeOH/DCM) to afford Intermediate 40 (1.3 g, 38%) as an off-white solid. MS (ESI) 834.6 [M+H]⁺.

### Intermediate 41

### Methyl 6-chloro-3-(3-hydroxypropyl)-7-(2-((((5-(((8-hydroxyquinolin-6-yl)thio)methyl)-1-methyl-1H-pyrazol-3-yl)methyl)(methyl)amino)methyl)-5,6-dihydro-4H-pyrrolo [1,2-b]pyrazol-3-yl)-1-methyl-1H-indole-2-carboxylate

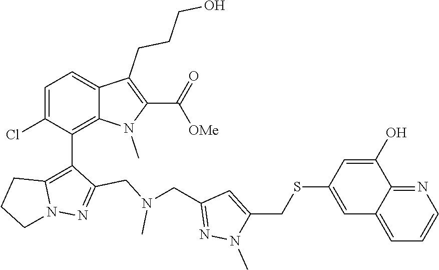 [SMILES: COC(=O)c1c(CCCO)c2ccc(Cl)c(-c3c(CN(C)Cc4cc(CSc5cc(O)c6ncccc6c5)n(C)n4)nn4c3CCC4)c2n1C]

Intermediate 41 was synthesized from Intermediate 40 following a procedure for the preparation of Intermediate 22. MS (LCMS) 714.5 [M+H]⁺.

### Intermediate 42

### Methyl (Z)-1⁶-chloro-1¹,6¹,4-trimethyl-2⁵,2⁶-dihydro-1¹H,2⁴H,6¹H-10-oxa-8-thia-4-aza-9(6,8)-quinolina-1(7,3)-indola-2(3,2)-pyrrolo[1,2-b]pyrazola-6(3,5)-pyrazolacyclotridecaphane-1²-carboxylate

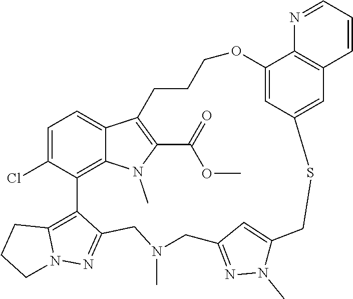 [SMILES: COC(=O)c1c2c3ccc(Cl)c(c3n1C)-c1c(nn3c1CCC3)CN(C)Cc1cc(n(C)n1)CSc1cc(c3ncccc3c1)OCCC2]

Intermediate 42 was synthesized from Intermediate 41 following a procedure for the preparation of Intermediate 23. MS (LCMS) 696.5 [M+H]⁺.

### Intermediate 43A

### (Rₐ)-(+)-Methyl (Z)-1⁶-chloro-1¹,6¹,4-trimethyl-2⁵,2⁶,9¹,9²,9³,94-hexahydro-1¹H,2⁴H,6¹H-10-oxa-8-thia-4-aza-9(6,8)-quinolina-1(7,3)-indola-2(3 ,2)-pyrrolo[1,2-b]pyrazola-6(3,5)-pyrazolacyclotridecaphane-1²-carboxylate

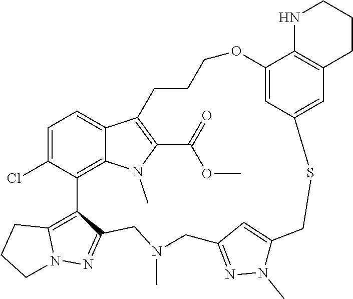

### Intermediate 43B

### (Sₐ)-(−)-Methyl (Z)-1⁶-chloro-1¹,6¹,4-trimethyl-2⁵,2⁶,9¹,9²,9³,9⁴-hexahydro-1¹H,2⁴H,6¹H-10-oxa-8-thia-4-aza-9(6,8)-quinolina-1(7,3)-indola-2(3,2)-pyrrolo[1,2-b]pyrazola-6(3,5)-pyrazolacyclotridecaphane-1²-carboxylate

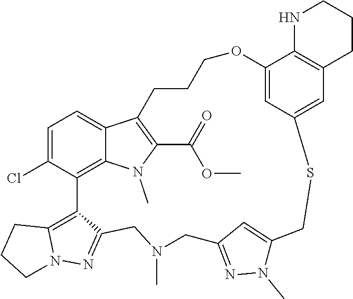

Intermediates 43A and 43B were synthesized from Intermediate 42 following a procedure for the preparation of Intermediates 24A and 24B to afford racemic methyl (Z)-1⁶-chloro-1¹,6¹,4-trimethyl-2⁵,2⁶,9¹,9²,9³,9⁴-hexahydro-1¹H,2⁴H,6¹H-10-oxa-8-thia-4-aza-9(6,8)-quinolina-1(7,3)-indola-2(3,2)-pyrrolo[1,2-b]pyrazola-6(3,5)-pyrazolacyclotridecaphane-1²-carboxylate (340 mg, 56%) as an off-white solid. The atropisomers were separated by chiral SFC chromatography (Lux Cellulose-2 (30×250 mm) column, 40% (0.2% 7M NH₃ in MeOH, CH₃CN:MeOH; 1:1)) to give peak 1 (Intermediate 43A, 130 mg) and peak 2 (Intermediate 43B, 120 mg). Intermediate 43A: off-white solid; 99.8% chiral purity; MS (LCMS) 700.4 [M+H]⁺. Intermediate 43B: off-white solid; 98.3% chiral purity; MS (LCMS) 700.5 [M+H]⁺. The absolute stereochemistry of Intermediate 43A and Intermediate 43B was arbitrarily assigned.

### Example 3A

### (Rₐ)-(+)-(Z)-1⁶-Chloro-1¹,6¹,4-trimethyl-2⁵,2⁶,9¹,9²,9³,9⁴-hexahydro-1¹H,2⁴H,6¹H-10-oxa-8-thia-4-aza-9(6,8)-quinolina-1(7,3)-indola-2(3,2)-pyrrolo[1,2-b]pyrazola-6(3,5)-pyrazolacyclotridecaphane-1²-carboxylic acid

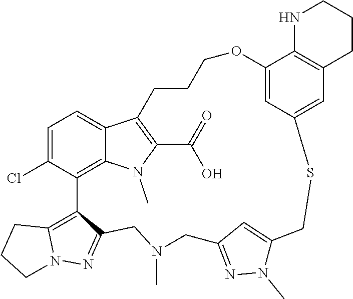

Example 3A was synthesized from Intermediate 43A following a procedure for the preparation of Example 1. Example 3A: (51 mg, 40%), white solid; 96.8% chiral purity; ¹H NMR (400 MHz, DMSO-d₆) δ 13.33 (br s, 1H), 10.10-9.20 (m, 1H), 8.00-7.80 (m, 1H), 7.20 (s, 1H), 6.50-6.30 (m, 2H), 5.30-5.00 (m, 2H), 4.60-3.33 (m, 17H), 3.33-3.00 (m, 3H), 2.90-2.40 (m, 9H), 2.20-2.00 (m 2H), 1.80-1.60 (m, 2H); MS (LCMS) 686.4[M+H]⁺.

### Example 3B

### (Sₐ)-(−)-(Z)-1⁶-Chloro-1¹,6¹,4-trimethyl-2⁵,2⁶,9¹,9²,9³,9⁴-hexahydro-1¹H,2⁴H,6¹H-10-oxa-8-thia-4-aza-9(6,8)-quinolina-1(7,3)-indola-2(3,2)-pyrrolo[1,2-b]pyrazola-6(3,5)-pyrazolacyclotridecaphane-1²-carboxylic acid

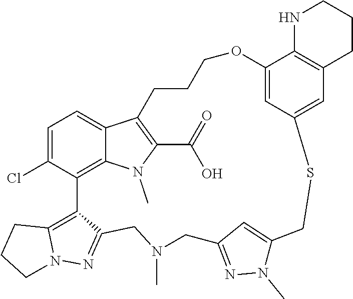

Example 3B was synthesized from Intermediate 43B following a procedure for the preparation of Example 1. Example 3B: (55 mg, 47%), white solid; 98.5% chiral purity; ¹H NMR (400 MHz, DMSO-d₆) δ 13.36 (br s, 1H), 10.10-9.20 (m, 1H), 8.00-7.80 (m, 1H), 7.21 (d, J=8.4 Hz, 1H), 6.50-6.30 (m, 2H), 5.30-5.05 (m, 2H), 4.40-3.80 (m, 7H), 3.75-3.40 (m, 10H), 3.33-3.00 (m, 3H), 2.90-2.50 (m, 9H), 2.30-2.15 (m 2H), 1.80-1.80 (m, 2H); MS (LCMS) 686.4[M+H]⁺. The absolute stereochemistry of Example 3A and Example 3B was arbitrarily assigned.

### Intermediate 44

### Methyl 3-(3-acetoxypropyl)-6-chloro-7-(2-formyl-4,5,6,7-tetrahydropyrazolo[1,5-a]pyridin-3-yl)-1-methyl-1H-indole-2-carboxylate

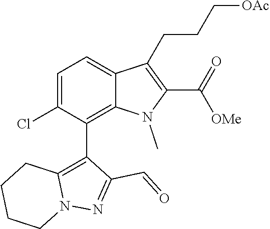 [SMILES: COC(=O)c1c(CCCOC(C)=O)c2ccc(Cl)c(-c3c(C=O)nn4c3CCCC4)c2n1C]

Intermediate 44 was synthesized from Intermediate 31 following a procedure from the preparation of Intermediate 38. MS (LCMS) 472.3 [M+H]⁺.

### Intermediate 45

### Methyl 3-(3-acetoxypropyl)-6-chloro-1-methyl-7-(2-((methylamino)methyl)-4,5,6,7-tetrahydropyrazolo[1,5-a]pyridin-3-yl)-1H-indole-2-carboxylate

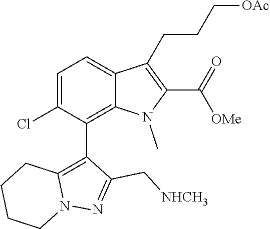 [SMILES: CNCc1nn2c(c1-c1c(Cl)ccc3c(CCCOC(C)=O)c(C(=O)OC)n(C)c13)CCCC2]

Intermediate 45 was synthesized from Intermediate 44 following a procedure for the preparation of Intermediate 39. MS (LCMS) 487.5 [M+H]⁺.

### Intermediate 46

### Methyl 6-chloro-3-(3-hydroxypropyl)-7-(2-((((5-(((8-((4-methoxybenzyl)oxy)quinolin-6-yl)thio)methyl)-1-methyl-1H-pyrazol-3-yl)methyl)(methyl)amino)methyl)-4,5,6,7-tetrahydropyrazolo[1,5-a]pyridin-3-yl)-1-methyl-1H-indole-2-carboxylate

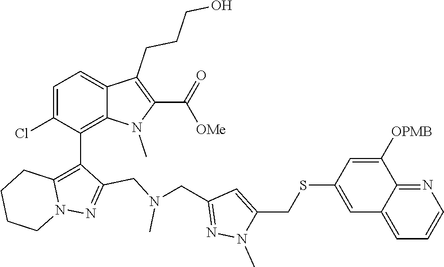

To a stirred solution of Intermediate 45 (1.0 g, 2.05 mmol) in DMF (5 mL) at 0° C. was added K₂CO₃ (485 mg, 3.509 mmol) and Intermediate 10 (0.990 mg, 2.26 mmol). The ice bath was removed, and the reaction was stirred at rt for 16 h. Two equivalent reactions were run simultaneously. The reactions were combined and diluted with water (100 mL). The mixture was extracted with EtOAc (2×100 mL). The combined organic layers were dried (Na₂SO₄), filtered and evaporated. The residue was purified by flash chromatography (SiO₂, MeOH/DCM) to afford methyl 3-(3-acetoxypropyl)-6-chloro-7-(2-((((5-(((8-((4-methoxybenzyl)oxy)quinolin-6-yl)thio)-methyl)-1-methyl-1H-pyrazol-3-yl)methyl)(methyl)amino)methyl)-4,5,6,7-tetrahydropyrazolo-[1,5-a]pyridin-3-yl)-1-methyl-1H-indole-2-carboxylate (1.9 g, 30%) as a white solid. MS (LCMS) 890.6 [M+H]⁺.

To a stirred solution of methyl 3-(3-acetoxypropyl)-6-chloro-7-(2-((((5-(((8-((4-methoxybenzyl)oxy)quinolin-6-yl)thio)methyl)-1-methyl-1H-pyrazol yl)methyl)(methyl)-amino)methyl)-4,5,6,7-tetrahydropyrazolo[1,5-a]pyridin-3-yl)-1-methyl-1H-indole-2-carboxylate (950 mg, 1.067 mmol) in MeOH (10 mL) was added K₂CO₃ (294 mg 2.14 mmol) at 0° C. The ice bath was removed, and the reaction was stirred at rt for 2 h. Two equivalent reactions were run simultaneously. The reaction mixtures were combined and diluted with water (50 mL) and DCM (100 mL). The organic layer was separated, dried (Na₂SO₄), filtered and evaporated. The compound was dried under high vacuum to provide Intermediate 46 (1.8 g) as a brown solid. MS (LCMS) 848.7 [M+H]⁺.

### Intermediate 47

### Methyl 6-chloro-3-(3-hydroxypropyl)-7-(2-((((5-(((8-hydroxyquinolin-6-yl)thio)methyl)-1-methyl-1H-pyrazol-3-yl)methyl)(methyl)amino)methyl)-4,5,6,7-tetrahydropyrazolo[1,5-a]pyridin-3-yl)-1-methyl-1H-indole-2-carboxylate

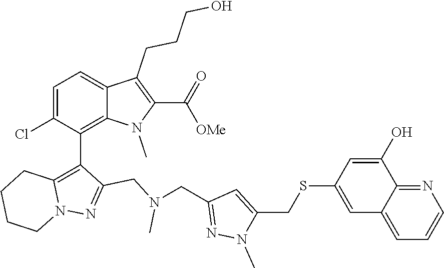 [SMILES: COC(=O)c1c(CCCO)c2ccc(Cl)c(-c3c(CN(C)Cc4cc(CSc5cc(O)c6ncccc6c5)n(C)n4)nn4c3CCCC4)c2n1C]

Intermediate 47 was synthesized from Intermediate 46 following a procedure for the preparation of Intermediate 22. MS (LCMS) 728.3 [M+H]⁺.

### Intermediate 48

### Methyl (Z)-1⁶-chloro-1¹,6¹,4-trimethyl-2⁴,2⁵,2⁶,2⁷-tetrahydro-1¹H,6¹H-10-oxa-8-thia-4-aza-9(6,8)-quinolina-2(3,2)-pyrazolo[1,5-a]pyridina-1(7,3)-indola-6(3,5)-pyrazolacyclotridecaphane-1²-carboxylate

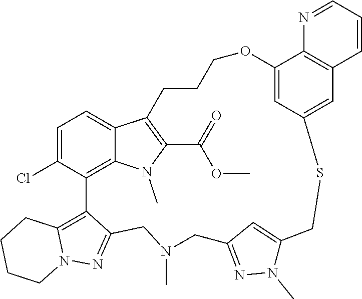 [SMILES: COC(=O)c1c2c3ccc(Cl)c(c3n1C)-c1c(nn3c1CCCC3)CN(C)Cc1cc(n(C)n1)CSc1cc(c3ncccc3c1)OCCC2]

Intermediate 48 was synthesized from Intermediate 47 following a procedure of the preparation of Intermediate 23. MS (LCMS) 710.5 [M+H]⁺.

### Intermediate 49A

### (Rₐ)-(+)-Methyl (Z)-1⁶-chloro-1¹,6¹,4-trimethyl-2⁴,2⁵,2⁶,2⁷,9¹,9²,9³9⁴-octahydro-1¹H,6¹H-10-oxa-8-thia-4-aza-9(6,8)-quinolina-2(3,2)-pyrazolo[1,5-a]pyridina-1(7,3)-indola-6(3,5)-pyrazolacyclotridecaphane-1²-carboxylate

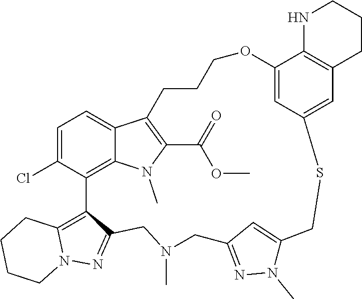

### Intermediate 49B

### (Sₐ)-(−)-Methyl (Z)-1⁶-chloro-1¹,6¹,4-trimethyl-2⁴,2⁵,2⁶,2⁷,9¹,9²,9³,9⁴-octahydro-1¹H,6¹H-10-oxa-8-thia-4-aza-9(6,8)-quinolina-2(3,2)-pyrazolo[1,5-a]pyridina-1(7,3)-indola-6(3,5)-pyrazolacyclotridecaphane-1²-carboxylate

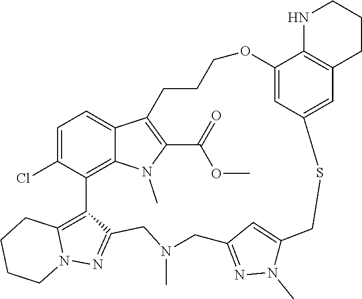

Intermediates 49A and 49B were synthesized from Intermediate 48 following a procedure for the preparation of Intermediates 24A and 24B to afford racemic methyl (Z)-1⁶-chloro-1¹,6¹,4-trimethyl-2⁴,2⁵,2⁶,2⁷,9¹,9²,9³,9⁴-octahydro-1¹H,6¹H-10-oxa-8-thia-4-aza-9(6,8)-quinolina-2(3,2)-pyrazolo[1,5-a]pyridina-1(7,3)-indola-6(3,5)-pyrazolacyclo-tridecaphane-1²-carboxylate (130 mg) as an off-white solid. MS (LCMS) 714.3 [M+H]⁺. The atropisomers were separated by chiral SFC chromatography (Lux Cellulose-2 (30×250 mm) column, 40% (0.2% 7M NH₃ in MeOH, CH₃CN:MeOH; 1:1)) to give peak 1 (Intermediate 49A, 40 mg) and peak 2 (Intermediate 49B, 50 mg). Intermediate 49A: off-white solid; 99.9% chiral purity; MS (LCMS) 714.3 [M+H]⁺. Intermediate 49B: off-white solid; 99.7% chiral purity; MS (LCMS) 714.3 [M+H]⁺. The absolute stereochemistry of Intermediate 49A and Intermediate 49B was arbitrarily assigned.

### Example 4A

### (Rₐ)-(+)-(Z)-1⁶-Chloro-1¹,6¹,4-trimethyl-2⁴,2⁵,2⁶,2⁷,9¹,9²,9³,9⁴-octahydro-1¹H,6¹H-10-oxa-8- thia-4-aza-9(6,8)-quinolina-2(3,2)-pyrazolo[1,5-a]pyridina-1(7,3)-indola-6(3,5)-pyrazolacyclotridecaphane-1²-carboxylic acid

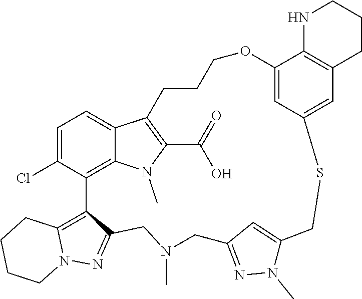

Example 4A was synthesized from Intermediate 49A following a procedure for the preparation of Example 1. Example 4A: (29 mg, 74%), white solid; 98.6% chiral purity; ¹H NMR (400 MHz, DMSO-d6) δ 13.32 (s, 1H), 10.10-9.20 (m, 1H), 7.90 (d, J=8.4 Hz, 1H), 7.25 (d, J=8.4 Hz, 1H), 6.43-6.35 (m, 2H), 5.62-5.55 (m, 2H), 4.50-3.50 (m, 18H), 3.10-2.90 (m, 4H), 2.70-2.60 (m, 1H), 2.40-2.20 (m, 3H), 2.10-1.60 (m, 9H); MS (LCMS) 700.3 [M+H]⁺.

### Example 4B

### (Sₐ)-(−)-(Z)-1⁶-Chloro-1¹,6¹,4-trimethyl-2⁴,2⁵,2⁶,2⁷,9¹,9²,9³,9⁴-octahydro-1¹H,6¹H-10-oxa-8- thia-4-aza-9(6,8)-quinolina-2(3,2)-pyrazolo[1,5-a]pyridina-1(7,3)-indola-6(3,5)-pyrazolacyclotridecaphane-1²-carboxylic acid

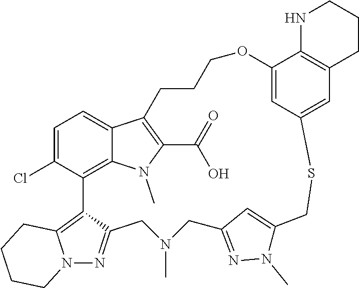

Example 4B was synthesized from Intermediate 49B following a procedure for the preparation of Example 1. Example 4B: (23.5 mg, 48%), white solid; 99.7% chiral purity; ¹H NMR (400 MHz, DMSO-d₆) δ 13.32 (s, 1H), 10.10-9.20 (m, 1H), δ 7.93 (m, 1H), 7.25 (d, J=8.0 Hz, 1H), 6.46-6.34 (m, 2H), 5.07 (m, 2H), 4.20 (s, 2H), 4.09-3.93 (m, 16H), 3.33-3.03 (m, 4H), 2.65-2.70 (m, 3H), 2.42-2.00 (m, 6H), 1.50-1.90 (m, 4H); MS (LCMS) 700.1 [M−H]⁺. The absolute stereochemistry of Example 4A and Example 4B was arbitrarily assigned.

### Intermediate 50

### 3-(((tert-Butyldiphenylsilyl)oxy)methyl)-5-ethynyl-1-methyl-1H-pyrazole

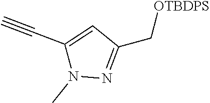

To a stirred solution of ethyl 3-(((tert-butyldiphenylsilyl)oxy)methyl)-1H-pyrazole-5-carboxylate (250 g, 613 mmol) in THF (2.5 L) were added Cs₂CO₃ (239 g, 735 mmol) and MeI (95.7 g, 674 mmol) at 0° C. The ice bath was removed, and the reaction was stirred at rt for 16 h. Upon completion by TLC, the reaction was quenched with water (2 L) and extracted with EtOAc (2×2 L). The combined organic layers were washed with brine (3 L), dried (Na₂SO₄), filtered and concentrated under reduced pressure. The residue was purified by flash chromatography (SiO₂, EtOAc/Pet. ether) to afford ethyl 3-(((tert-butyldiphenylsilyl)oxy)methyl)-1-methyl-1H-pyrazole-5-carboxylate (130 g, 50%) as an oil. MS (LCMS) 423.4 [M+H]⁺.

To a stirred solution of ethyl 3-(((tert-butyldiphenylsilyl)oxy)methyl)-1-methyl-1H-pyrazole-5-carboxylate (120 g, 284 mmol) in THF (1.2 L) was added 2.4 M LiAlH₄ in THF (118 mL, 284 mmol) at 0° C. The ice bath was removed, and the reaction was stirred at rt for 2 h. Upon completion by TLC, the reaction mixture was cooled to 0° C. and quenched by the dropwise addition of water (20 mL) followed by 15% NaOH (20 mL). The mixture was diluted with water (50 mL) and EtOAc (1 L). The mixture was filtered through a Celite pad and the pad was washed with EtOAc (1 L). The organic layer was separated, washed with brine (2 L), filtered, dried (Na₂SO₄), and concentrated under reduced pressure. The residue was purified by flash chromatography (SiO₂, 40% EtOAc/Pet. ether) to afford (3-(((tert-butyldiphenylsilyl)oxy)-methyl)-1-methyl-1H-pyrazol-5-yl)methanol (81 g, 75%) as an oil. MS (LCMS) 381.3 [M+H]⁺.

To a stirred solution of (3-(((tert-butyldiphenylsilyl)oxy)methyl)-1-methyl-1H-pyrazol-5-yl)methanol (80.0 g, 210 mmol) in THF (800 mL) was added MnO₂ (201.2 g, 2.32 mol) at 0° C. The reaction was stirred at rt for 60 h. The reaction was filtered through a Celite pad and washed with EtOAc (1 L). The filtrate was concentrated under reduced pressure and the residue was purified by flash chromatography (SiO₂, 10% EtOAc/Pet. ether) to afford 3-(((tert-butyldiphenylsilyl)oxy)methyl)-1-methyl-1H-pyrazole-5-carbaldehyde (36 g, 45%) as an oil. MS (LCMS) 379.5 [M+H]⁺.

To a stirred solution of 3-(((tert-butyldiphenylsilyl)oxy)methyl)-1-methyl-1H-pyrazole-5-carbaldehyde (36.0 g, 95.2 mmol) in THF (360 mL) was added dimethyl (1-diazo-2-oxopropyl)phosphonate (27.44 g, 142.8 mmol) and K₂CO₃ (39.42 g, 285.7 mmol) at 0° C. The ice bath was removed, and the reaction was stirred at rt for 1 h. Upon completion by LCMS, the reaction was quenched with water (100 mL) and concentrated under reduced pressure. The residue was diluted with water (200 mL) and extracted with EtOAc (2×200 mL). The combined organic layers were dried (Na₂SO₄), filtered and concentrated under reduced pressure. The residue was purified by flash chromatography (SiO₂, 10% EtOAc/Pet. ether) to afford Intermediate 50 (32 g, 90%) as an oil. MS (LCMS) 375.5 [M+H]+.

### Intermediate 51

### 6-((3-(((tert-Butyldiphenylsilyl)oxy)methyl)-1-methyl-1H-pyrazol-5-yl)ethynyl)-8-((4-methoxybenzyl)oxy)quinoline

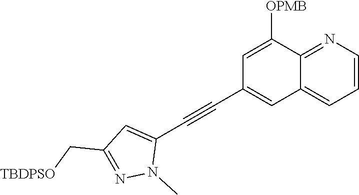

To the degassed solution of Intermediate 8 (26.0 g, 75.8 mmol) in DMF (260 mL) were added Intermediate 50 (36.85 g, 98.54 mmol), CuI (1.44 g, 7.58 mmol), Pd(PPh₃)₂Cl₂ (5.32 g, 7.58 mmol), and TEA (42.5 mL, 303 mmol). The reaction mixture was degassed for 10 minutes and then heated at 90° C. for 16 h. The reaction was diluted with water (200 mL) and extracted with EtOAc (3×200 mL). The combined organic layers were washed with brine (2×100 mL), dried (Na₂SO₄), filtered and concentrated under reduced pressure. The residue was purified by flash chromatography (SiO₂, 40% EtOAc/Pet. ether) to afford Intermediate 51 (40 g, 82%) as an oil. MS (LCMS) 638.4 [M+H]⁺.

### Intermediate 52

### (5-(2-(8-((4-Methoxybenzyl)oxy)quinolin-6-yl)ethyl)-1-methyl-1H-pyrazol-3-yl)methanamine

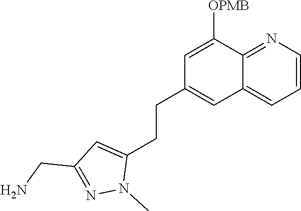

To a stirred solution of Intermediate 51 (40.0 g, 62.8 mmol) in THF (400 mL) was added 1.0 M TBAF in THF (62.8 mL, 62.8 mmol) at 0° C. The ice bath was removed, and the reaction was stirred at rt for 1 h. Upon completion by TLC, the reaction was diluted with EtOAc (300 mL) and washed with water (200 mL) and brine (200 mL). The organic layer was dried (Na₂SO₄), filtered, and concentrated under reduced pressure. The residue was purified by flash chromatography (SiO₂, 40% EtOAc/Pet. ether) to afford (5-((8-((4-methoxybenzyl)oxy)quinolin-6-yl)ethynyl)-1-methyl-1H-pyrazol-3-yl)methanol (19 g, 75%) as a yellow solid. MS (LCMS) 400.3 [M+H]⁺.

To a stirred solution of (5-((8-((4-methoxybenzyl)oxy)quinolin-6-yl)ethynyl)-1-methyl-1H-pyrazol-3-yl)methanol (19.0 g, 47.6 mmol) in DMF (190 mL) were added 2,6-lutidine (25.5 g, 238 mmol) and LiCl (12.1 g, 286 mmol). MsCl (10.9 g, 95.2 mmol) was added dropwise over 10 min and the reaction was stirred at rt for 16 h. The reaction was diluted with water (200 mL) and extracted with EtOAc (2×150 mL). The combined organic layers were dried (Na₂SO₄), filtered and concentrated under reduced pressure. The residue was purified by flash chromatography (SiO₂, 60% EtOAc/Pet. ether) to afford 6-((3-(chloromethyl)-1-methyl-1H-pyrazol-5-yl)ethynyl)-8-((4-methoxybenzyl)oxy)quinoline (17 g, 85%) as a yellow solid. MS (LCMS) 418.3 [M+H]⁺.

To a stirred solution of 6-((3-(chloromethyl)-1-methyl-1H-pyrazol-5-yl)ethynyl)-8-((4-methoxybenzyl)oxy)quinoline (17.0 g, 40.8 mmol) in dry MeCN (170 mL) were added NaN₃ (13.25 g, 203.8 mmol) and KI (6.77 g, 40.8 mmol) at rt. The reaction was stirred at 85° C. for 2 h. The reaction was cooled to rt, filtered and concentrated. The residue was dissolved in EtOAc (250 mL), washed with water (100 mL) and brine (100 mL). The organic layer was dried (Na₂SO₄), filtered and concentrated under reduced pressure. The residue was triturated with EtOAc, filtered and dried under vacuum to afford 6-((3-(azidomethyl)-1-methyl-1H-pyrazol-5-yl)ethynyl)-8-((4-methoxybenzyl)oxy)quinoline (13 g, 72%) as an off-white solid. MS (LCMS) 425.3[M+H]⁺.

To a stirred solution of 6-((3-(azidomethyl)-1-methyl-1H-pyrazol-5-yl)ethynyl)-8-((4-methoxybenzyl)oxy)quinoline (13.0 g, 30.6 mmol) in dry MeOH:DCM (2:1, 195 mL) was added 10 wt. % Pd/C (8 g) under an argon atmosphere. The reaction was stirred at rt for 24 h under 1 atm H₂. Upon completion by TLC, the reaction was filtered through a Celite pad. The pad was washed with 10% MeOH/DCM and the filtrate was concentrated under reduced pressure. The residue was purified by reverse phase HPLC (C18, 20% H₂O:CH₃CN) to afford Intermediate 52 (5 g, 40%) as a yellow solid. MS (LCMS) 403.3 [M+H]⁺.

### Intermediate 53

### Methyl 3-(3-acetoxypropyl)-6-chloro-7-(2-((((5-(2-(8-((4-methoxybenzyl)oxy)quinolin-6-yl)ethyl)-1-methyl-1H-pyrazol-3-yl)methyl)amino)methyl)-5,6-dihydro-4H-pyrrolo [1,2-b]pyrazol-3-yl)-1-methyl-1H-indole-2-carboxylate

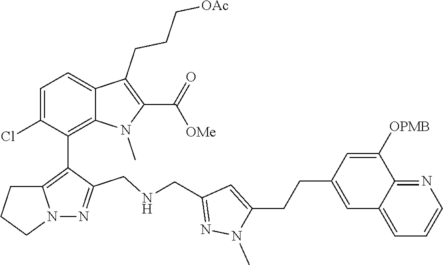

To a stirred solution of Intermediate 38 (1.20 g, 2.63 mmol) in MeOH (12 mL) were added Intermediate 52 (1.06 g, 2.63 mmol) and TEA (0.55 mL, 3.94 mmol) at rt. The reaction was stirred at rt for 16 h. NaBH₄ (199 mg, 5.25 mmol) was added at 0° C. portionwise. The ice bath was removed, and the reaction was stirred at rt for 16 h. Upon completion by LCMS, the solvent was evaporated, and the reaction was diluted with DCM (50 mL). The mixture washed with water (20 mL) and brine (20 mL). The organic layer was dried (Na₂SO₄), filtered and concentrated under reduced pressure. The residue was purified by flash chromatography (SiO₂, 2% MeOH/DCM) to afford Intermediate 53 (1 g, 45%) as a brown solid. MS (ESI) 844.7 [M+H]⁺.

### Intermediate 54

### Methyl 6-chloro-3-(3-hydroxypropyl)-7-(2-((((5-(2-(8-hydroxyquinolin-6-yl)ethyl)-1-methyl-1H-pyrazol-3-yl)methyl)(methyl)amino)methyl)-5,6-dihydro-4H-pyrrolo[1,2-b]pyrazol-3-yl)-1-methyl-1H-indole-2-carboxylate

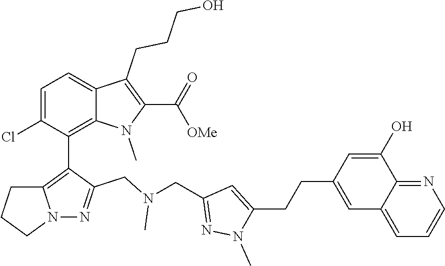 [SMILES: COC(=O)c1c(CCCO)c2ccc(Cl)c(-c3c(CN(C)Cc4cc(CCc5cc(O)c6ncccc6c5)n(C)n4)nn4c3CCC4)c2n1C]

To a stirred solution of Intermediate 53 (1.90 g, 2.25 mmol) in MeOH (20 mL) was added NaHCO₃ (947 mg, 11.3 mmol) at RT. The reaction was refluxed for 4 h. Upon completion by LCMS, the reaction was diluted with DCM (30 mL) and filtered through Celite. The pad was washed with DCM (50 mL) and the filtrate was concentrated under reduced pressure to afford methyl 6-chloro-3-(3-hydroxypropyl)-7-(2-((((5-(2-(8-((4-methoxybenzyl)oxy)quinolin-6-yl)ethyl)-1-methyl-1H-pyrazol-3-yl)methyl)amino)methyl)-5,6-dihydro-4H-pyrrolo[1,2-b]pyrazol-3-yl)-1-methyl-1H-indole-2-carboxylate (1.7 g) as off-white solid. MS (ESI) 802.6 [M+H]⁺.

To a stirred solution of methyl 6-chloro-3-(3-hydroxypropyl)-7-(2-((((5-(2-(8-((4-methoxybenzyl)oxy)quinolin-6-yl)ethyl)-1-methyl-1H-pyrazol-3-yl)methyl)amino)methyl)-5,6-dihydro-4H-pyrrolo[1,2-b]pyrazol-3-yl)-1-methyl-1H-indole-2-carboxylate (1.70 g, 2.12 mmol) in DCM (17 mL) was added TFA (1.62 mL, 21.2 mmol) at 0° C. The ice bath was removed, and the reaction was stirred at rt for 1.5 h. Upon completion by LCMS, the solvent was evaporated, and sat. NaHCO₃ (50 mL) was added. The mixture was extracted with DCM (2×80 mL). The combined organic layers were washed with brine (100 mL), dried (Na₂SO₄), filtered and concentrated. The residue was purified by flash chromatography (SiO₂, 4% MeOH/DCM) to afford methyl 6-chloro-3-(3-hydroxypropyl)-7-(2-((((5-(2-(8-hydroxyquinolin-6-yl)ethyl)-1-methyl-1H-pyrazol-3-yl) methyl)amino)methyl)-5,6-dihydro-4H-pyrrolo[1,2-b]pyrazol-3-yl)-1-methyl-1H-indole-2-carboxylate (1.43 g, 95%) as an off-white solid. MS (ESI) 682.6 [M+H]⁺.

To a stirred solution of methyl 6-chloro-3-(3-hydroxypropyl)-7-(2-((((5-(2-(8-hydroxyquinolin-6-yl)ethyl)-1-methyl-1H-pyrazol-3-yl)methyl)amino)methyl)-5,6-dihydro-4H-pyrrolo[1,2-b]pyrazol-3-yl)-1-methyl-1H-indole-2-carboxylate (1.00 g, 1.47 mmol) in DCE (10 mL) were added 37% aq. formaldehyde (0.086 mL, 3.14 mmol), NaOAc (154 mg, 1.88 mmol), and Na(OAc)3BH (154 mg, 1.88 mmol) at 0° C. The ice bath was removed, and the reaction was stirred at rt for 2 h. Upon completion by LCMS, the reaction was quenched with sat. aq. NaHCO₃ (20 mL). The mixture was extracted with EtOAc (2×30 mL). The combined organic layers were washed with brine (50 mL), dried (Na₂SO₄), filtered, concentrated. Two reactions of equivalent scale were run simultaneously and combined for purification. The residue was purified by flash chromatography (SiO₂, 2% MeOH/DCM) to afford Intermediate 54 (1 g, 65%) as a white solid. MS (ESI) 696.6 [M+H]⁺.

### Intermediate 55

### Methyl (Z)-1⁶-chloro-1¹,6¹,4-trimethyl-2⁵,2⁶-dihydro-1¹H,2⁴H,6¹H-10-oxa-4-aza-9(6,8)-quinolina-1(7,3)-indola-2(3,2)-pyrrolo[1,2-b]pyrazola-6(3,5)-pyrazolacyclotridecaphane-12-carboxylate

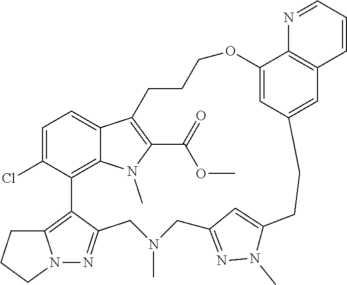 [SMILES: COC(=O)c1c2c3ccc(Cl)c(c3n1C)-c1c(nn3c1CCC3)CN(C)Cc1cc(n(C)n1)CCc1cc(c3ncccc3c1)OCCC2]

Intermediate 55 was synthesized from Intermediate 54 following a procedure for the preparation of Intermediate 23. MS (LCMS) 678.4 [M+H]⁺.

### Intermediate 56A

### (Rₐ)-(+)-Methyl (Z)-1⁶-chloro-1¹,6¹,4-trimethyl-2⁵,2⁶,9¹,9²,9³,9⁴-hexahydro-1¹H,2⁴H,6¹H-10-oxa-4-aza-9(6,8)-quinolina-1(7,3)-indola-2(3,2)-pyrrolo[1,2-b]pyrazola-6(3,5)-pyrazolacyclotridecaphane-1²-carboxylate

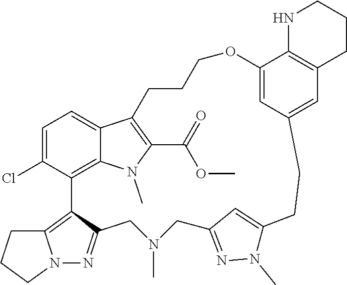

### Intermediate 56B

### (Sₐ)-(−)-Methyl (Z)-1⁶-chloro-1¹,6¹,4-trimethyl-2⁵,2⁶,9¹,9²,9³,9⁴-hexahydro-1¹H,2⁴H,6¹H-10-oxa-4-aza-9(6,8)-quinolina-1(7,3)-indola-2(3,2)-pyrrolo[1,2-b]pyrazola-6(3,5)-pyrazolacyclotridecaphane-1²-carboxylate

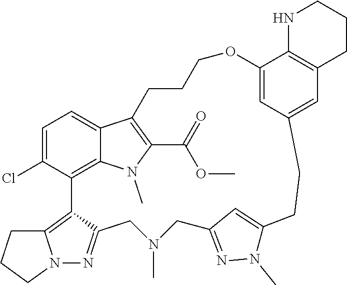

Intermediates 56A and 56B were synthesized from Intermediate 55 following a procedure for the preparation of Intermediates 24A and 24B to give racemic methyl (Z)-1⁶-chloro-1¹,6¹,4-trimethyl-2⁵,2⁶,9¹,9²,9³,9⁴-hexahydro-1¹H,2⁴H,6 ¹H-10-oxa-4-aza-9(6,8)-quinolina-1(7,3)-indola-2(3,2)-pyrrolo[1,2-b]pyrazola-6(3,5)-pyrazolacyclotridecaphane-1²-carboxylate (200 mg). The atropisomers were separated by chiral SFC chromatography (Chiralcel OD-H (30×250 mm) column, 40% (0.2% 7M methanolic NH₃ in MeOH)) to give peak 1 (Intermediate 56A, 38 mg) and peak 2 (Intermediate 56B, 36 mg). Intermediate 56A: brown solid; 96.9% chiral purity; MS (ESI) 682.6 [M+H]⁺; Intermediate 56B: brown solid; 99.0% chiral purity; MS (ESI) 682.6 [M+H]⁺. The absolute stereochemistry of Intermediate 56A and Intermediate 56B was arbitrarily assigned.

### Example 5A

### (Rₐ)-(+)-(Z)-1⁶-Chloro-1¹,6¹,4-trimethyl-2⁵,2⁶,9¹,9²,9³,9⁴-hexahydro-1¹H,2⁴H,6¹H-10-oxa-4-aza-9(6,8)-quinolina-1(7,3)-indola-2(3,2)-pyrrolo[1,2-b]pyrazola-6(3,5)-pyrazolacyclotridecaphane-1²-carboxylic acid

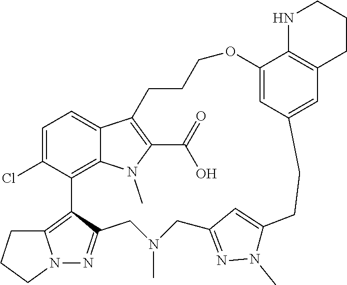

Example 5A was synthesized from Intermediate 56A following a procedure for the preparation of Example 1. Example 5A: (22 mg, 60%), off-white solid; 99.9% chiral purity; ¹H NMR (400 MHz, DMSO-d₆) δ 13.40 (br s, 1H), 7.75 (d, J=8.4 Hz, 1H), 7.16 (d, J=8.4 Hz, 1H), 6.25 (s, 1H), 6.16 (s, 1H), 4.58 (br s, 2H), 4.20-4.05 (m, 2H), 3.95-3.85 (m, 1H), 3.58 (s, 4H), 3.50-3.33 (m, 5H), 3.15-3.05 (m, 2H), 3.00-2.50 (m, 14H), 2.20-2.10 (m, 1H), 2.09-1.95 (m, 1H), 1.70 (s, 3H), 1.70-1.60 (m, 2H); MS (ESI) 668.5 [M+H]⁺.

### Example 5B

### (Sₐ)-(−)-(Z)-1⁶-Chloro-1¹,6¹,4-trimethyl-2⁵,2⁶,9¹,9²,9³,9⁴-hexahydro-1¹H,2⁴H,6¹H-10-oxa-4-aza-9(6,8)-quinolina-1(7,3)-indola-2(3,2)-pyrrolo[1,2-b]pyrazola-6(3,5)-pyrazolacyclotridecaphane-1²-carboxylic acid

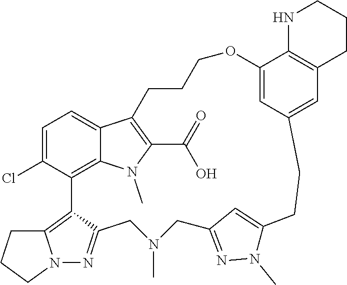

Example 5B was synthesized from Intermediate 56B following a procedure for the preparation of Example 1. Example 5B: (18 mg, 51%), off-white solid; 99.9% chiral purity; ¹H NMR (400 MHz, DMSO-d₆) δ 13.40 (br s, 1H), 7.79 (d, J=8.8 Hz, 1H), 7.18 (d, J=8.4 Hz, 1H), 6.24 (s, 1H), 6.17 (s, 1H), 4.57 (s, 2H), 4.20-4.10 (m, 2H), 3.95-3.85 (m, 1H), 3.58 (s, 4H), 3.50-3.33 (m, 5H), 3.15-3.05 (m, 2H), 3.00-2.50 (m, 14H), 2.25-2.15 (m, 1H), 2.10-1.95 (m, 1H), 1.70 (s, 3H), 1.70-1.60 (m, 2H); MS (ESI) 668.5 [M+H]⁺. The absolute stereochemistry of Example 5A and Example 5B was arbitrarily assigned.

### Intermediate 57

### Methyl 3-(3-acetoxypropyl)-6-chloro-7-(2-((((5-(2-(8-((4-methoxybenzyl)oxy)quinolin-6-yl)ethyl)-1-methyl-1H-pyrazol-3-yl)methyl)amino)methyl)-4,5,6,7-tetrahydropyrazolo[1,5-a]pyridin-3-yl)-1-methyl-1H-indole-2-carboxylate

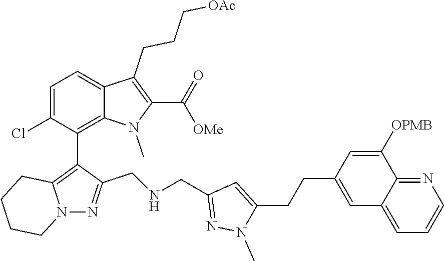

Intermediate 57 was synthesized from Intermediate 44 and Intermediate 52 following a procedure for the preparation of Intermediate 53. MS (LCMS) 858.6 [M+H]⁺.

### Intermediate 58

### Methyl 6-chloro-3-(3-hydroxypropyl)-7-(2-((((5-(2-(8-hydroxyquinolin-6-yl)ethyl)-1-methyl-1H-pyrazol-3-yl)methyl)(methyl)amino)methyl)-4,5,6,7-tetrahydropyrazolo[1,5-a]pyridin-3-yl)-1-methyl-1H-indole-2-carboxylate

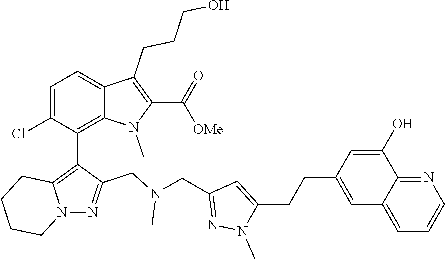 [SMILES: COC(=O)c1c(CCCO)c2ccc(Cl)c(-c3c(CN(C)Cc4cc(CCc5cc(O)c6ncccc6c5)n(C)n4)nn4c3CCCC4)c2n1C]

Intermediate 58 was prepared from Intermediate 57 following a procedure for the preparation of Intermediate 54. MS (LCMS) 710.6 [M+H]⁺.

### Intermediate 59

### Methyl (Z)-1⁶-chloro-1¹,6¹,4-trimethyl-2⁴,2⁵,2⁶,2⁷-tetrahydro-1¹H,6¹H-10-oxa-4-aza-9(6,8)-quinolina-2(3,2)-pyrazolo[1,5-a]pyridina-1(7,3)-indola-6(3,5)-pyrazolacyclotridecaphane-1²-carboxylate

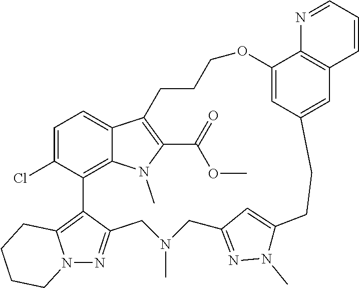 [SMILES: COC(=O)c1c2c3ccc(Cl)c(c3n1C)-c1c(nn3c1CCCC3)CN(C)Cc1cc(n(C)n1)CCc1cc(c3ncccc3c1)OCCC2]

Intermediate 59 was prepared from Intermediate 58 following a procedure for the preparation of Intermediate 23. MS (ESI) 692.6 [M+H]⁺.

### Intermediate 60A

### (Rₐ)-(+)-Methyl (Z)-1⁶-chloro-1¹,6¹4-trimethyl-2⁴,2⁵,2⁶,2⁷,9¹,9²,9³,9⁴-octahydro-1¹H,6¹H-10-oxa-4-aza-9(6,8)-quinolina-2(3,2)-pyrazolo[1,5-a]pyridina-1(7,3)-indola-6(3,5)-pyrazolacyclotridecaphane-1²-carboxylate

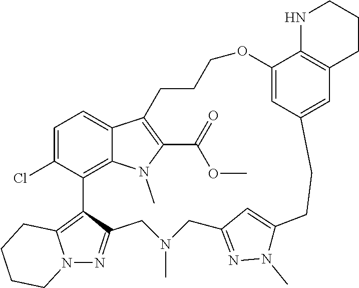

### Intermediate 60B

### (Sₐ)-(−)-Methyl (Z)-1⁶-chloro-1¹,6¹,4-trimethyl-2⁴,2⁵,2⁶,2⁷,9¹,9²,9³,9⁴-octahydro-1¹H,6¹H-10-oxa-4-aza-9(6,8)-quinolina-2(3,2)-pyrazolo[1,5-a]pyridina-1(7,3)-indola-6(3,5)-pyrazolacyclotridecaphane-1²-carboxylate

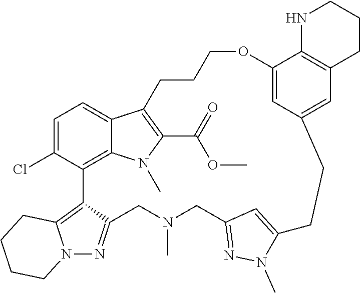

Intermediates 60A and 60B were synthesized from Intermediate 59 following a procedure for the preparation of Intermediates 24A and 24B to give racemic methyl (Z)-1⁶-chloro-1¹,6¹,4-trimethyl-2⁴,2⁵,2⁶,2⁷,9¹,9²,9³,9⁴-octahydro-1¹H,6¹H-10-oxa-4-aza-9(6,8)-quinolina-2(3,2)-pyrazolo[1,5-a]pyridina-1(7,3)-indola-6(3,5)-pyrazolacyclotridecaphane-1²-carboxylate (180 mg). The atropisomers were separated by chiral SFC chromatography (Chiralcel OD-H (30×250 mm) column, 50% (0.2% 7M Methanolic NH₃ in MeOH)) to give peak 1 (Intermediate 60A, 70 mg) and peak 2 (Intermediate 60B, 70 mg). Intermediate 60A: off-white solid; 98.0% chiral purity; MS (ESI) 696.1 [M+H]⁺. Intermediate 60B: off-white solid; 99.9% chiral purity; MS (ESI) 696.1 [M+H]⁺. The absolute stereochemistry of Intermediate 60A and Intermediate 60B was arbitrarily assigned.

### Example 6A

### (Rₐ)-(+)-(Z)-1⁶-Chloro-1¹,6¹,4-trimethyl-2⁴,2⁵,2⁶,2⁷,9¹,9²,9³,9⁴-octahydro-1¹H,6¹H-10-oxa-4- aza-9(6,8)-quinolina-2(3,2)-pyrazolo[1,5-a]pyridina-1(7,3)-indola-6(3,5)-pyrazolacyclotridecaphane-1²-carboxylic acid

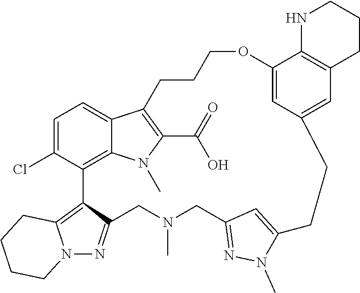

Example 6A was synthesized from Intermediate 60A following a procedure for the preparation of Example 1. Example 6A: (25 mg, 37%), off-white solid; 98.3% chiral purity; ¹H NMR (400 MHz, DMSO-d₆) δ 13.35 (br s, 1H), 10.10-9.50 (m, 1H), 7.95-7.85 (m, 1H), 7.30-7.20 (m, 1H), 6.35-6.20 (m, 1H), 6.00 (s, 1H), 5.18 (s, 1H), 4.72 (s, 1H), 4.30-3.33 (m, 14H), 3.30-2.50 (m, 11H), 2.50-2.40 (m, 4H), 2.25-2.15 (m, 1H), 2.10-1.98 (m, 3H), 1.90-1.60 (m, 4H); MS (ESI) 682.6 [M+H]+.

### Example 6B

### (Sₐ)-(−)-(Z)-1⁶-Chloro-1¹,6¹,4-trimethyl-2⁴,2⁵,2⁶,2⁷,9¹,9²,9³,9⁴-octahydro-1¹H,6¹H-10-oxa-4- aza-9(6,8)-quinolina-2(3,2)-pyrazolo[1,5-a]pyridina-1(7,3)-indola-6(3,5)-pyrazolacyclotridecaphane-1²-carboxylic acid

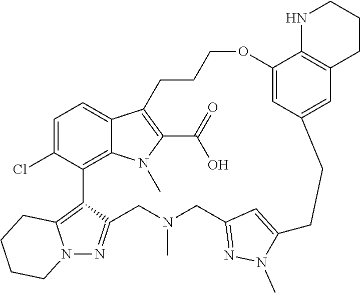

Example 6B was synthesized from Intermediate 60B following a procedure for the preparation of Example 1. Example 6B: (29 mg, 42%), off-white solid; 99.9% chiral purity; ¹H NMR (400 MHz, DMSO-d₆) δ 13.3 (br s, 1H), 10.01-9.40 (m, 1H), 7.95-7.85 (m, 1H), 7.30-7.20 (m, 1H), 6.35-6.20 (m, 1H), 6.00 (s, 1H), 5.17 (s, 1H), 4.72 (s, 1H), 4.30-3.65 (m, 6H), 3.60-3.33 (m, 9H), 3.30-2.50 (m, 12H), 2.50-2.40 (m, 2H), 2.25-2.15 (m, 1H), 2.10-1.98 (m, 3H), 1.90-1.60 (m, 4H); MS (ESI) 682.6 [M+H]⁺. The absolute stereochemistry of Example 6A and Example 6B was arbitrarily assigned.

### Intermediate 61

### Methyl 7-bromo-6-fluoro-3-(3-methoxy-3-oxopropyl)-1H-indole-2-carboxylate

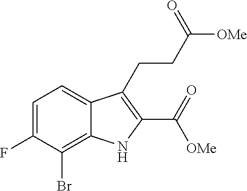 [SMILES: COC(=O)CCc1c(C(=O)OC)[nH]c2c(Br)c(F)ccc12]

Intermediate 61 was synthesized from 2-bromo-3-fluoroaniline following a procedure for the preparation of Intermediate 1. MS (LCMS) 358.1[M+H]⁺.

### Intermediate 62

### Methyl 3-(3-acetoxypropyl)-7-bromo-6-fluoro-1-methyl-1H-indole-2-carboxylate

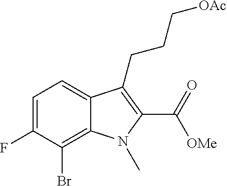 [SMILES: COC(=O)c1c(CCCOC(C)=O)c2ccc(F)c(Br)c2n1C]

Intermediate 62 was synthesized from Intermediate 61 following procedures for the preparation of Intermediates 2-4. MS (LCMS) 386.2 [M+H]⁺.

### Intermediate 63

### Methyl 3-(3-acetoxypropyl)-6-fluoro-7-(2-(((4-methoxybenzyl)oxy)methyl)-5,6-dihydro-4H-pyrrolo[1,2-b]pyrazol-3-yl)-1-methyl-1H-indole-2-carboxylate

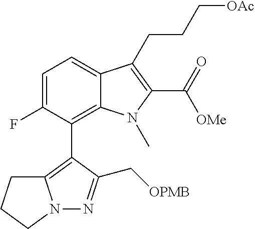

Intermediate 63 was synthesized from Intermediate 62 and Intermediate 16 following a procedure for the preparation of Intermediate 17. MS (LCMS) 564.5 [M+H]⁺.

### Intermediate 64

### Methyl 3-(3-acetoxypropyl)-6-fluoro-7-(2-(hydroxymethyl)-5,6-dihydro-4H-pyrrolo[1,2-b]pyrazol-3-yl)-1-methyl-1H-indole-2-carboxylate

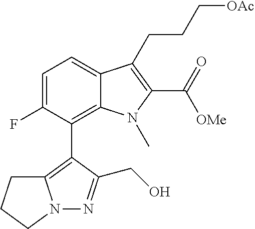 [SMILES: COC(=O)c1c(CCCOC(C)=O)c2ccc(F)c(-c3c(CO)nn4c3CCC4)c2n1C]

Intermediate 64 was synthesized from Intermediate 63 following a procedure for the preparation of Intermediate 18. MS (LCMS) 444.4 [M+H]⁺.

### Intermediate 65

### Methyl 3-(3-acetoxypropyl)-7-(2-(chloromethyl)-5,6-dihydro-4H-pyrrolo[1,2-b]pyrazol-3-yl)-6-fluoro-1-methyl-1H-indole-2-carboxylate

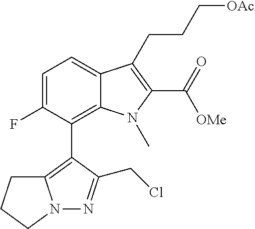 [SMILES: COC(=O)c1c(CCCOC(C)=O)c2ccc(F)c(-c3c(CCl)nn4c3CCC4)c2n1C]

Intermediate 65 was prepared from Intermediate 64 following a procedure for the preparation of Intermediate 19. MS (LCMS) 462.4 [M+H]⁺.

### Intermediate 66

### Methyl 3-(3-acetoxypropyl)-6-fluoro-7-(2-(iodomethyl)-5,6-dihydro-4H-pyrrolo[1,2-b]pyrazol-3-yl)-1-methyl-1H-indole-2-carboxylate

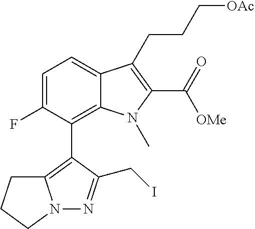 [SMILES: COC(=O)c1c(CCCOC(C)=O)c2ccc(F)c(-c3c(CI)nn4c3CCC4)c2n1C]

Intermediate 66 was prepared from Intermediate 65 following a procedure for the preparation of Intermediate 20. MS (LCMS) 554.4 [M+H]⁺.

### Intermediate 67

### Methyl 6-fluoro-3-(3-hydroxypropyl)-7-(2-((((5-(((8-((4-methoxybenzyl)oxy)quinolin-6-yl)thio)methyl)-1-methyl-1H-pyrazol-3-yl)methyl)thio)methyl)-5,6-dihydro-4H-pyrrolo [1,2-b]pyrazol-3-yl)-1-methyl-1H-indole-2-carboxylate

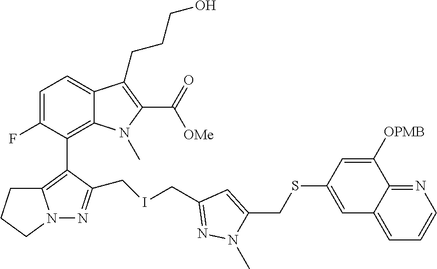

Intermediate 67 was synthesized from Intermediate 66 and Intermediate 11 following a procedure for the preparation of Intermediate 21. MS (LCMS) 821.6 [M+H]⁺.

### Intermediate 68

### Methyl 6-fluoro-3-(3-hydroxypropyl)-7-(2-((((5-(((8-hydroxyquinolin-6-yl)thio)methyl)-1-methyl-1H-pyrazol-3-yl)methyl)thio)methyl)-5,6-dihydro-4H-pyrrolo[1,2-b]pyrazol-3-yl)-1-methyl-1H-indole-2-carboxylate

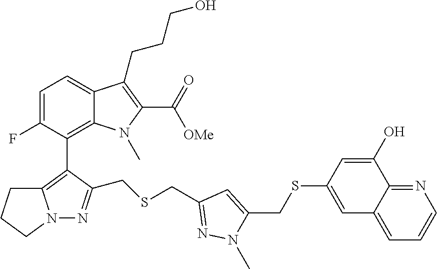 [SMILES: COC(=O)c1c(CCCO)c2ccc(F)c(-c3c(CSCc4cc(CSc5cc(O)c6ncccc6c5)n(C)n4)nn4c3CCC4)c2n1C]

Intermediate 68 was synthesized from Intermediate 67 following a procedure for the preparation of Intermediate 22. MS (LCMS) 701.4 [M+H]⁺.

### Intermediate 69

### Methyl (Z)-1⁶-fluoro-1¹,6¹-dimethyl-2⁵,2⁶-dihydro-1¹H,2⁴H,6¹H-10-oxa-4,8-dithia-9(6,8)-quinolina-1(7,3)-indola-2(3,2)-pyrrolo[1,2-b]pyrazola-6(3,5)-pyrazolacyclotridecaphane carboxylate

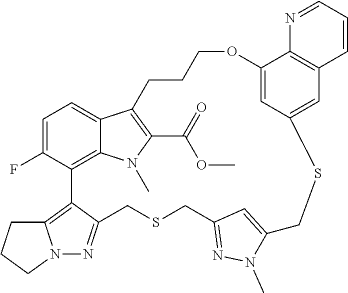 [SMILES: COC(=O)c1c2c3ccc(F)c(c3n1C)-c1c(nn3c1CCC3)CSCc1cc(n(C)n1)CSc1cc(c3ncccc3c1)OCCC2]

Intermediate 69 was synthesized from Intermediate 68 following a procedure for the preparation of Intermediate 23. MS (LCMS) 683.5 [M+H]⁺.

### Intermediate 70A

### (Rₐ)-(+)-Methyl (Z)-1⁶-fluoro-1¹,6¹-dimethyl-2⁵,2⁶,9¹,9²,9³,9⁴-hexahydro-1¹H,2⁴H,6¹H-10-oxa-4,8-dithia-9(6,8)-quinolina-1(7,3)-indola-2(3,2)-pyrrolo[1,2-b]pyrazola-6(3,5)-pyrazolacyclotridecaphane-1²-carboxylate

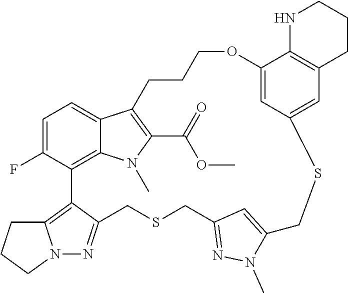 [SMILES: COC(=O)c1c2c3ccc(F)c(c3n1C)-c1c(nn3c1CCC3)CSCc1cc(n(C)n1)CSc1cc3c(c(c1)OCCC2)NCCC3]

### Intermediate 70B

### (Sₐ)-(−)-Methyl (Z)-1⁶-fluoro-1¹,6¹-dimethyl-2⁵,2⁶,9¹,9²,9³,9⁴-hexahydro-1¹H,2⁴H,6¹H-10-oxa-4,8-dithia-9(6,8)-quinolina-1(7,3)-indola-2(3,2)-pyrrolo[1,2-b]pyrazola-6(3,5)-pyrazolacyclotridecaphane-1²-carboxylate

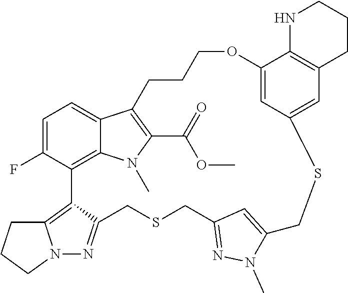

Intermediates 70A and 70B were synthesized from Intermediate 69 following a procedure for the preparation of Intermediates 24A and 24B to give racemic methyl (Z)-1⁶-fluoro-1¹,6¹-dimethyl-2⁵,2⁶,9¹,9²,9³,9⁴-hexahydro-1¹H,2⁴H,6¹H-10-oxa-4,8-dithia-9(6,8)-quinolina-1(7,3)-indola-2(3,2)-pyrrolo[1,2-b]pyrazola-6(3,5)-pyrazolacyclotridecaphane-1²-carboxylate (160 mg, 31%). The atropisomers were separated by chiral SFC chromatography (Chiralpak IC (30×250 mm) column, 45% (0.2% 7M Methanolic NH₃ in CH₃CN:MeOH; 1:1)) to give peak 1 (Intermediate 70A, 45 mg) and peak 2 (Intermediate 70B, 21 mg). Intermediate 70A: off-white solid; 96.1% chiral purity; MS (ESI) 687.5 [M+H]⁺. Intermediate 70B: off-white solid; 98.9% chiral purity; MS (ESI) 687.6 [M+H]⁺. The absolute stereochemistry of Intermediate 70A and Intermediate 70B was arbitrarily assigned.

### Example 7A

### (Rₐ)-(+)-(Z)-1⁶-Fluoro-1¹,6¹-dimethyl-2⁵,2⁶,9¹,9²,9³,9⁴-hexahydro-1¹H,2⁴H,6¹H-10-oxa-4,8-dithia-9(6,8)-quinolina-1(7,3)-indola-2(3,2)-pyrrolo[1,2-b]pyrazola-6(3,5)-pyrazolacyclotridecaphane-1²-carboxylic acid

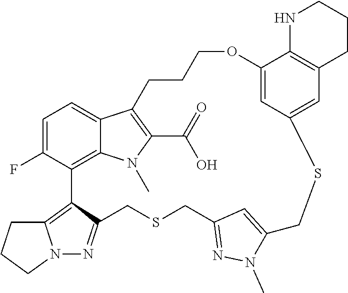

Example 7A was synthesized from Intermediate 70A following a procedure for the preparation of Example 1. Example 7A: (31 mg, 70%), off-white solid; 72.6% chiral purity; 1H NMR (400 MHz, DMSO-d₆) δ 13.20 (br s, 1H), 7.82-7.75 (m, 1H), 6.94 (t, J=9.2 Hz, 1H), 6.55 (s, 1H), 6.16 (s, 1H), 5.20 (br s, 1H) 4.82 (s, 1H), 4.20-4.05 (m, 2H), 4.00-3.80 (m, 2H), 3.70-3.55 (m, 8H), 3.50-3.30 (m, 3H) 3.29-3.15 (m, 2H), 3.12-3.00 (m, 2H), 2.75-2.67 (m, 3H), 2.65-2.50 (m, 4H), 2.25-2.12 (m, 1H), 2.10-1.95 (m, 1H), 1.80-1.70 (m, 2H); MS (LCMS) 673.5 [M+H]⁺.

### Example 7B

### (Sₐ)-(−)-(Z)-1⁶-Fluoro-1¹,6¹-dimethyl-2⁵,2⁶,9¹,9²,9³,9⁴-hexahydro-1¹H,2⁴H,6¹H-10-oxa-4,8-dithia-9(6,8)-quinolina-1(7,3)-indola-2(3,2)-pyrrolo[1,2-b]pyrazola-6(3,5)-pyrazolacyclotridecaphane-1²-carboxylic acid

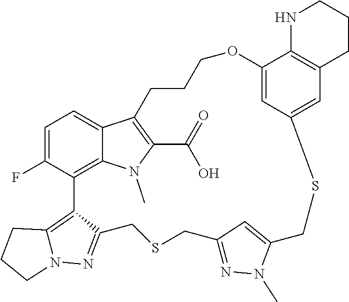

Example 7B was synthesized from Intermediate 70B following a procedure for the preparation of Example 1. Example 7B: (31 mg, 72%), off-white solid; 73.4% chiral purity; 1H NMR (400 MHz, DMSO-d6) δ 13.20 (br s, 1H), 7.82-7.75 (m, 1H), 6.93 (t, J=9.2 Hz, 1H), 6.54 (s, 1H), 6.14 (s, 1H), 5.2 (br s, 1H), 4.82 (s, 1H), 4.20-4.05 (m, 2H), 3.98-3.80 (m, 2H), 3.70-3.55 (m, 8H), 3.50-3.30 (m, 3H), 3.30-3.15 (m, 2H), 3.15-2.95 (m, 2H), 2.75-2.50 (m, 7H), 2.25-2.12 (m, 1H), 2.10-1.95 (m, 1H), 1.80-1.70 (m, 2H); MS (LCMS) 673.5 [M+H]⁺. The absolute stereochemistry of Example 7A and Example 7B was arbitrarily assigned.

### Intermediate 71

### Methyl 3-(3-acetoxypropyl)-6-fluoro-7-(2-(((4-methoxybenzyl)oxy)methyl)-4,5,6,7-tetrahydropyrazolo[1,5-a]pyridin-3-yl)-1-methyl-1H-indole-2-carboxylate

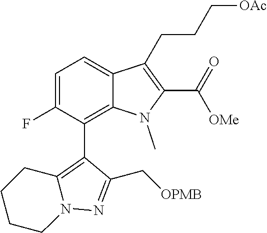

Intermediate 71 was synthesized from Intermediate 62 and Intermediate 29 following a procedure for the preparation of Intermediate 17. MS (LCMS) 578.5 [M+H]⁺.

### Intermediate 72

### Methyl 3-(3-acetoxypropyl)-6-fluoro-7-(2-(hydroxymethyl)-4,5,6,7-tetrahydropyrazolo[1,5-a]pyridin-3-yl)-1-methyl-1H-indole-2-carboxylate

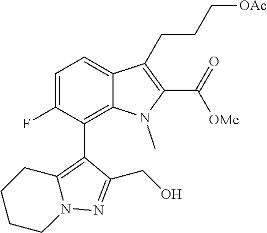 [SMILES: COC(=O)c1c(CCCOC(C)=O)c2ccc(F)c(-c3c(CO)nn4c3CCCC4)c2n1C]

Intermediate 72 was synthesized from Intermediate 71 following a procedure for the preparation of Intermediate 18. MS (LCMS) 458.4 [M+H]⁺.

### Intermediate 73

### Methyl 3-(3-acetoxypropyl)-7-(2-(chloromethyl)-4,5,6,7-tetrahydropyrazolo[1,5-a]pyridin-3-yl)-6-fluoro-1-methyl-1H-indole-2-carboxylate

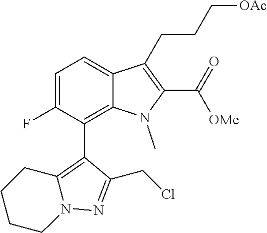 [SMILES: COC(=O)c1c(CCCOC(C)=O)c2ccc(F)c(-c3c(CCl)nn4c3CCCC4)c2n1C]

Intermediate 73 was synthesized from Intermediate 72 following a procedure for the preparation of Intermediate 19. MS (LCMS) 476.2 [M+H]⁺.

### Intermediate 74

### Methyl 3-(3-acetoxypropyl)-6-fluoro-7-(2-(iodomethyl)-4,5,6,7-tetrahydropyrazolo[1,5-a]pyridin-3-yl)-1-methyl-1H-indole-2-carboxylate

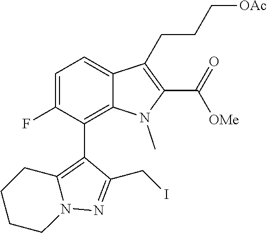 [SMILES: COC(=O)c1c(CCCOC(C)=O)c2ccc(F)c(-c3c(CI)nn4c3CCCC4)c2n1C]

Intermediate 74 was synthesized from Intermediate 73 following a procedure for the preparation of Intermediate 20. MS (LCMS) 568.5 [M+H]⁺.

### Intermediate 75

### Methyl 6-fluoro-3-(3-hydroxypropyl)-7-(2-((((5-(((8-((4-methoxybenzyl)oxy)quinolin-6-yl)thio)methyl)-1-methyl-1H-pyrazol-3-yl)methyl)thio)methyl)-4,5,6,7-tetrahydropyrazolo[1,5-a]pyridin-3-yl)-1-methyl-1H-indole-2-carboxylate

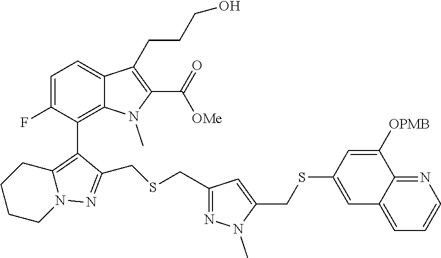

Intermediate 75 was synthesized from Intermediate 74 and Intermediate 11 following a procedure for the preparation of Intermediate 21. MS (LCMS) 835.5 [M+H]⁺.

### Intermediate 76

### Methyl 6-fluoro-3-(3-hydroxypropyl)-7-(2-((((5-(((8-hydroxyquinolin-6-yl)thio)methyl)-1-methyl-1H-pyrazol-3-yl)methyl)thio)methyl)-4,5,6,7-tetrahydropyrazolo[1,5-a]pyridin-3-yl)-1-methyl-1H-indole-2-carboxylate

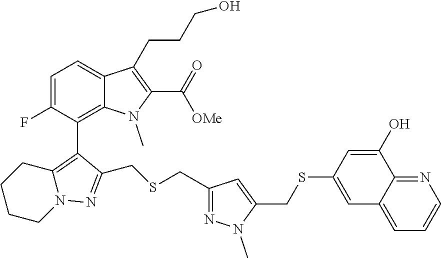 [SMILES: COC(=O)c1c(CCCO)c2ccc(F)c(-c3c(CSCc4cc(CSc5cc(O)c6ncccc6c5)n(C)n4)nn4c3CCCC4)c2n1C]

Intermediate 76 was synthesized from Intermediate 75 following a procedure for the preparation of Intermediate 22. MS (LCMS) 715.6 [M+H]⁺.

### Intermediate 77

### Methyl (Z)-1⁶-fluoro-1¹,6¹-dimethyl-2⁴,2⁵,2⁶,2⁷-tetrahydro-1¹H,6¹H-10-oxa-4,8-dithia-9(6,8)-quinolina-2(3,2)-pyrazolo[1,5-a]pyridina-1(7 ,3)-indola-6(3,5)-pyrazolacyclotridecaphane-1²-carboxylate

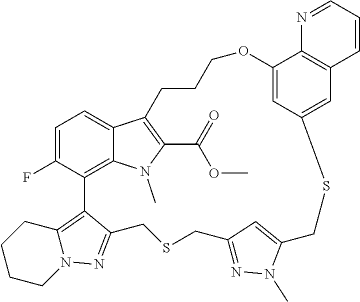 [SMILES: COC(=O)c1c2c3ccc(F)c(c3n1C)-c1c(nn3c1CCCC3)CSCc1cc(n(C)n1)CSc1cc(c3ncccc3c1)OCCC2]

Intermediate 77 was synthesized from Intermediate 76 following a procedure for the preparation of Intermediate 23. MS (LCMS) 697.4 [M+H]⁺.

### Intermediate 78A

### (Rₐ)-(+)-(Z)-1⁶-fluoro-1¹,6¹-dimethyl-2⁴,2⁵,2⁶,2⁷,9¹,9²,9³,9⁴-octahydro-11H,61H-10-oxa-4,8-dithia-9(6,8)-quinolina-2(3,2)-pyrazolo[1,5-a]pyridina-1(7,3)-indola-6(3,5)-pyrazolacyclotridecaphane-1²-carboxylate

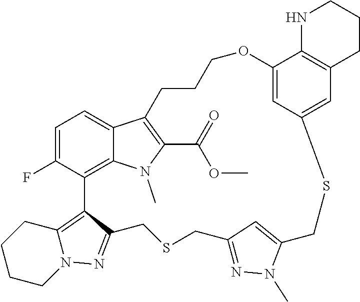

### Intermediate 78B

### (Sₐ)-(−)-Methyl (Z)-1⁶-fluoro-1¹,6¹-dimethyl-2⁴,2⁵,2⁶,2⁷,9¹,9²,9³,9⁴-octahydro-1¹H,6¹H-10-oxa-4,8-dithia-9(6,8)-quinolina-2(3,2)-pyrazolo[1,5-a]pyridina-1(7,3)-indola-6(3,5)-pyrazolacyclotridecaphane-1²-carboxylate

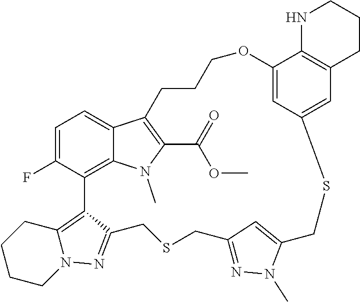

Intermediates 78A and 78B were synthesized from Intermediate 77 following a procedure for the preparation of Intermediates 24A and 24B to give racemic methyl (Z)-1⁶-fluoro-1¹,6¹-dimethyl-2⁴,2⁵,2⁶,2⁷,9¹,9²,9³,9⁴-octahydro-1¹H,6¹H-10-oxa-4,8-dithia-9(6,8)-quinolina-2(3,2)-pyrazolo[1,5-a]pyridina-1(7,3)-indola-6(3,5)-pyrazolacyclotridecaphane-1²-carboxylate (500 mg, 77%). The atropisomers were separated by chiral SFC chromatography (Chiralcel OX-3 (30×250 mm) column, 40% (0.2% 7M Methanolic NH₃ in CH₃CN:MeOH; 1:1)) to give peak 1 (Intermediate 78A, 90 mg) and peak 2 (Intermediate 78B, 60 mg). Intermediate 78A: off-white solid; 99.2% chiral purity; MS (LCMS) 701.4 [M+H]⁺. Intermediate 78B: off-white solid; 98.0% chiral purity; MS (LCMS) 701.5 [M+H]⁺. The absolute stereochemistry of Intermediate 78A and Intermediate 78B was arbitrarily assigned.

### Example 8A

### (Rₐ)-(+)-(Z)-1⁶-Fluoro-1¹,6¹-dimethyl-2⁴,2⁵,2⁶,2⁷,9¹,9²,9³,9⁴-octahydro-1¹H,6 ¹H-10-oxa-4,8- dithia-9(6, 8)-quinolina-2(3,2)-pyrazolo[1,5-a]pyridina-1(7,3)-indola-6(3,5)-pyrazolacyclotridecaphane-1²-carboxylic acid

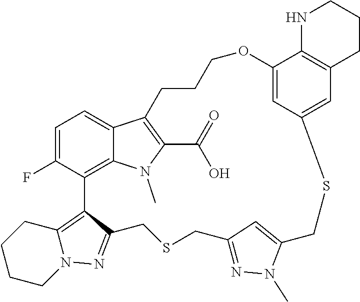

Example 8A was synthesized from Intermediate 78A following a procedure for the preparation of Example 1. Example 8A: (61 mg, 69%), yellow solid; 99.9% chiral purity; ¹H NMR (400 MHz, DMSO-d₆) δ 13.2 (brs, 1H),7.87 (dd, J=8.4 Hz, 5.6 Hz, 1H), 6.97 (t, J=8.0 Hz, 1H), 6.60 (s, 1H), 6.37 (brs, 1H), 4.78 (s, 1H), 4.10-4.01 (m, 5H), 3.64-3.59 (m, 7H), 3.46 (d, J=12.8 Hz, 1H), 3.34-3.06 (m, 6H), 2.81 (d, J=14.0 Hz, 1H), 2.65-2.50 (m, 2H), 2.45-1.97 (m, 6H), 1.80-1.75 (m, 4H); MS (LCMS) 687.5 [M+H]⁺.

### Example 8B

### (Sₐ)-(−)-(Z)-1⁶-Fluoro-1¹,6¹-dimethyl-2⁴,2⁵,2⁶,2⁷,9¹,9²,9³,9⁴-octahydro-1¹H,6¹H-10-oxa-4,8- dithia-9(6, 8)-quinolina-2(3,2)-pyrazolo[1,5-a]pyridina-1(7,3)-indola-6(3,5)-pyrazolacyclotridecaphane-1²-carboxylic acid

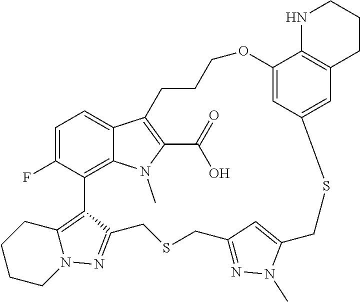

Example 8B was synthesized from Intermediate 78B following a procedure for the preparation of Example 1. Example 8B: (22.4 mg, 38%), yellow solid; 96.6% chiral purity; ¹H NMR (400 MHz, DMSO-d₆) δ 13.2 (brs, 1H), 7.87 (dd, J=8.4 Hz, 5.6 Hz, 1H), 6.98 (t, J=8.0 Hz, 1H), 6.61 (s, 1H), 6.40 (brs, 1H), 4.78 (s, 1H), 4.12-4.07 (m, 5H), 3.64-3.59 (m, 7H), 3.46 (d, J=12.8 Hz, 1H), 3.34-3.06 (m, 6H), 2.81 (d, J=14.0 Hz, 1H), 2.65-2.50 (m, 2H), 2.45-1.97 (m, 6H), 1.80-1.77 (m, 4H); MS (LCMS) 687.4 [M+H]⁺. The absolute stereochemistry of Example 8A and Example 8B was arbitrarily assigned.

### Intermediate 79

### Di-tert-butyl 1-(bicyclo[1.1.1]pentan-1-yl)hydrazine-1,2-dicarboxylate

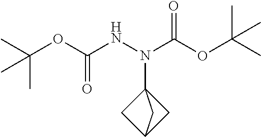 [SMILES: CC(C)(C)OC(=O)NN(C(=O)OC(C)(C)C)C12CC(C1)C2]

To a stirred solution of 1,1-dibromo-2,2-bis(chloromethyl)cyclopropane (25.0 g, 85.3 mmol) in n-pentane (100 mL) was added 1.6 M MeLi in Et₂O (126 mL, 202 mmol) at −45° C. The reaction was stirred for 15 min at −45° C. The temperature was raised to 0° C., and the reaction was stirred for 2 h. The solution was distilled by collecting the distillate at −78° C. under reduced pressure to get tricyclo[1.1.1.0^{1,3}]pentane (160 mL, 0.5 M in Et₂O). ¹H NMR (400 MHz, CDCl₃) δ 2.02 (s, 6H).

Mn(dpm)₃ (54.0 g, 90.9 mmol) was dissolved in 2-proponal (500 mL) and the reaction was cooled to −15° C. A solution of di-tert-butyl (E)-diazene-1,2-dicarboxylate (156.8 g, 681.0 mmol) and phenyl silane (49.0 g, 454 mmol) in DCM (500 mL) was added to the reaction at −15° C. dropwise over 30 min. Next, a solution of tricyclo[1.1.1.0^{1,3}]pentane (0.62 M, 454 mmol, 30.0 g) was added to the reaction at −15° C. The reaction was warmed to rt and stirred for 24 h. The solvent was evaporated, and the residue was purified by flash chromatography (SiO₂, 5-20% EtOAc/Pet. ether) to afford Intermediate 79 (40 g, 29%) as a white solid. ¹H NMR (400 MHz, CDCl₃) δ 6.28 (br s, 1H), 2.39 (s, 1H), 2.04 (s, 6H), 1.47 (s, 18H).

### Intermediate 80

### Bicyclo[1.1.1]pentan-1-ylhydrazine dihydrochloride

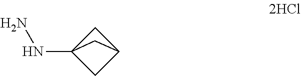

To a stirred solution of Intermediate 79 (40.0 g, 134 mmol) in EtOAc (80 mL) was added 4M HCl in 1,4-dioxane (400 mL) at 0° C. The reaction was stirred for 16 h at rt. The reaction was concentrated, triturated with pentane, and dried under high vacuum to afford Intermediate 80 (22 g, quantitative) as a white solid. ¹H NMR (300 MHz, DMSO-d₆) δ 7.05 (br s, 1H), 2.45 (s, 1H), 1.83 (s, 6H).

### Intermediate 81

### Ethyl 1-(bicyclo[1.1.1]pentan-1-yl)-3-methyl-1H-pyrazole-5-carboxylate

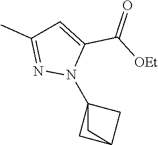 [SMILES: CCOC(=O)c1cc(C)nn1C12CC(C1)C2]

To a stirred solution of ethyl 2,4-dioxovalerate (40.0 g, 258 mmol) in ethanol (2200 mL) was added Intermediate 80 (22.0 g, 129 mmol) at 0° C. The reaction was stirred at 80° C. for 2 h. Upon completion, the solvent was evaporated, and the reaction was diluted with water (500 mL). The mixture was extracted with EtOAc (2×500 mL). The combined organic layers were washed with brine (750 mL), dried (Na₂SO₄), filtered and evaporated. The residue was purified by flash chromatography (SiO₂, 5-20% EtOAc/Pet. ether) to afford ethyl 1-(bicyclo[1.1.1]pentan-1-yl)-5-methyl-1H-pyrazole-3-carboxylate (20 g, 48%) and the desired Intermediate 81 (10 g, 35%). ¹H NMR (400 MHz, CDCl₃) δ 6.62 (s, 1H), 4.37-4.28 (m, 2H), 2.55 (s, 1H), 2.42 (s, 6H), 2.26 (s, 3H), 1.38-1.34 (m, 3H); MS (LCMS) 221.2 [M+H]⁺.

### Intermediate 82

### (1-(Bicyclo[1.1.1]pentan-1-yl)-3-methyl-1H-pyrazol-5-yl)methanol

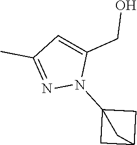

To a stirred solution of Intermediate 81 (4.50 g, 20.4 mmol) in THF (45 ml) was added 2.4 M LiAlH₄ in THF (8.52 mL, 20.4 mmol) at 0° C. The reaction was stirred at rt for 2 h. Two reactions of equal scale were run simultaneously. Upon completion by TLC, the reactions were quenched with cold sat. Na₂SO₄ (20 mL). The resulting slurries were filtered through Celite which was washed with EtOAc (4×200 mL). The filtrates were combined, washed with brine (500 mL), dried (Na₂SO₄), filtered and evaporated. The material was dried under high vacuum to afford Intermediate 82 (7.3 g, quant.) as an oil. ¹H NMR (400 MHz, CDCl₃) δ 5.99 (s, 1H), 4.67 (d, J=6.0 Hz, 2H), 2.57 (s, 1H), 2.38 (s, 6H), 2.24 (s, 3H); MS (LCMS) 179.4 [M+H]⁺.

### Intermediate 83

### (1-(Bicyclo[1.1.1]pentan-1-yl)-4-bromo-3-methyl-1H-pyrazol-5-yl)methanol

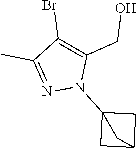

To a stirred solution of Intermediate 82 (7.30 g, 41.0 mmol) in DCM (100 mL) was added NBS (7.30 g, 41.0 mmol) at 0° C. The ice bath was removed, and the reaction was stirred at rt for 2 h. Upon completion by TLC, the reaction was diluted with water (100 mL) and extracted with DCM (3×100 mL). The combined organic layers were dried (Na₂SO₄), filtered and evaporated. The residue was triturated with pentane:ether (1:1) (3×50 mL) and dried under high vacuum to afford Intermediate 83 (8 g, 75%) as a yellow solid. ¹H NMR (400 MHz, CDCl₃) δ 4.44 (s, 2H), 2.56 (s, 1H), 2.28 (s, 6H), 2.09 (s, 3H); MS (LCMS) 257.6 [M+H]⁺.

### Intermediate 84

### 1-(Bicyclo[1.1.1]pentan-1-yl)-4-bromo-5-(((4-methoxybenzyl)oxy)methyl)-3-methyl-1H-pyrazole

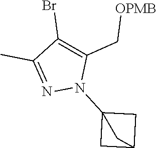

To a stirred solution of Intermediate 83 (4.00 g, 15.6 mmol) in DMF (80 mL) was added 60% NaH (0.933 g, 38.9 mmol) at 0° C. The reaction was stirred at 0° C. for 30 min and 1-(chloromethyl)-4-methoxybenzene (3.64 g, 23.3 mmol) and NaI (0.46 g, 3.11 mmol) were then added. The ice bath was removed, and the reaction was stirred at rt for 2 h. Two reactions of equal size were run simultaneously. Upon completion by TLC, the reactions were quenched with ice water (200 mL). The mixtures were combined and extracted with EtOAc (3×200 mL). The combined organic layers were washed with water (2×200 mL), brine (200 mL), dried (Na₂SO₄), filtered, and concentrated under reduced pressure. The residue was purified by flash chromatography (SiO₂, 7% EtOAc/Pet. ether) to afford Intermediate 84 (10 g, 85%) as an oil. ¹H NMR (400 MHz, CDCl₃) δ 7.27-7.23 (m, 2H), 6.90-6.85 (m, 2H), 4.54 (s, 2H), 4.42 (s, 2H), 3.81 (s, 3H), 2.53 (s, 1H), 2.34 (s, 6H), 2.24 (s, 3H); MS (LCMS) 377.1 [M+H]⁺.

### Intermediate 85

### 1-(Bicyclo[1.1.1]pentan-1-yl)-5-(((4-methoxybenzyl)oxy)methyl)-3-methyl-4-(4,4,5,5-tetramethyl-1,3,2-dioxaborolan-2-yl)-1H-pyrazole

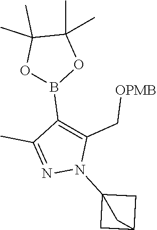

A suspension of Intermediate 84 (6.00 g, 15.9 mmol), bis(pinacolato)diboron (16.15 g, 63.83 mmol) and KOAc (5.47 g, 55.9 mmol) in DMA (100 mL) was degassed with argon for 15 min. Pd[P(Cy)₃]₂Cl₂ (589 mg, 0.800 mmol) was added and the reaction was degassed for 10 min. The reaction was stirred at 110° C. for 4 h. Two reactions of equal size were run simultaneously. Upon completion, the reactions were diluted with water (200 mL) and extracted with EtOAc (3×200 mL). The combined organic layers from both batches were washed with water (2×200 mL), brine (200 mL), dried (Na₂SO₄), filtered and concentrated under reduced pressure. The residue was purified by flash chromatography (SiO₂, 25-30% EtOAc/Pet. ether) to afford Intermediate 85 (9 g, 66%) as an oil. ¹H NMR (400 MHz, CDCl₃) δ 7.23 (d, J=8.8 Hz, 2H), 6.86 (d, J=8.8 Hz, 2H), 4.76 (s, 2H), 4.41 (s, 2H), 3.80 (s, 3H), 2.51 (s, 1H), 2.36 (s, 9H), 1.30-1.25 (m, 12H). MS (LCMS) 425.6 [M+H]⁺.

### Intermediate 86

### Methyl 3-(3-acetoxypropyl)-7-(1-(bicyclo[1.1.1]pentan-1-yl)-5-(((4-methoxybenzyl)oxy)methyl)-3-methyl-1H-pyrazol-4-yl)-6-chloro-1-methyl-1H-indole-2-carboxylate

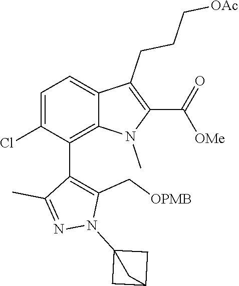

Intermediate 86 was prepared from Intermediate 85 and Intermediate 4 following a procedure for the preparation of Intermediate 17. MS (LCMS) 620.9 [M+H]⁺.

### Intermediate 87

### Methyl 3-(3-acetoxypropyl)-7-(1-(bicyclo[1.1.1]pentan-1-yl)-5-(hydroxymethyl)-3-methyl-1H-pyrazol-4-yl)-6-chloro-1-methyl-1H-indole-2-carboxylate

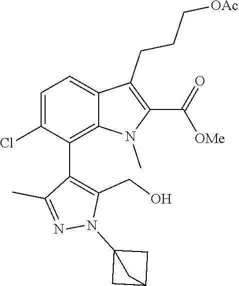

Intermediate 87 was prepared from Intermediate 86 following a procedure for the preparation of Intermediate 18. MS (LCMS) 500.5 [M+H]⁺.

### Intermediate 88

### Methyl 3-(3-acetoxypropyl)-7-(1-(bicyclo[1.1.1]pentan-1-yl)-5-(chloromethyl)-3-methyl-1H-pyrazol-4-yl)-6-chloro-1-methyl-1H-indole-2-carboxylate

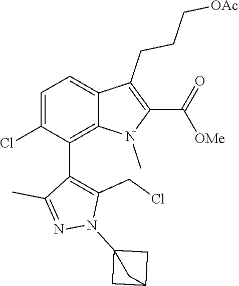

Intermediate 88 was prepared from Intermediate 87 following a procedure for the preparation of Intermediate 19. MS (LCMS) 518.3 [M+H]⁺.

### Intermediate 89

### Methyl 3-(3-acetoxypropyl)-7-(1-(bicyclo[1.1.1]pentan-1-yl)-5-(iodomethyl)-3-methyl-1H-pyrazol-4-yl)-6-chloro-1-methyl-1H-indole-2-carboxylate

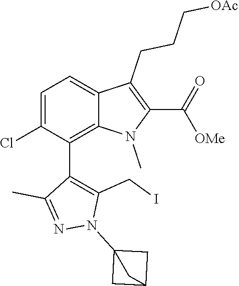

Intermediate 89 was prepared from Intermediate 88 following a procedure for the preparation of Intermediate 20. MS (LCMS) 610.8 [M+H]⁺.

### Intermediate 90

### Methyl 7-(1-(bicyclo[1.1.1]pentan-1-yl)-5-((((5-(((8-((4-methoxybenzyl)oxy)quinolin-6-yl)thio)methyl)-1-methyl-1H-pyrazol-3-yl)methyl)thio)methyl)-3-methyl-1H-pyrazol-4-yl)-6-chloro-3-(3-hydroxypropyl)-1-methyl-1H-indole-2-carboxylate

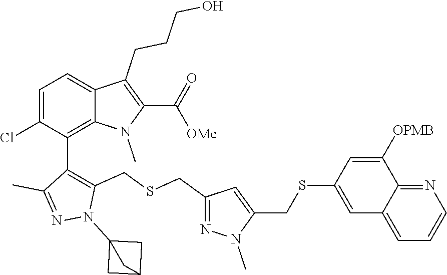

Intermediate 90 was synthesized from Intermediate 89 and Intermediate 11 following a procedure for the preparation of Intermediate 21. MS (LCMS) 877.3 [M+H]⁺.

### Intermediate 91

### Methyl 7-(1-(bicyclo[1.1.1]pentan-1-yl)-5-((((5-(((8-hydroxyquinolin-6-yl)thio)methyl)-1-methyl-1H-pyrazol-3-yl)methyl)thio)methyl)-3-methyl-1H-pyrazol-4-yl)-6-chloro-3-(3-hydroxypropyl)-1-methyl-1H-indole-2-carboxylate

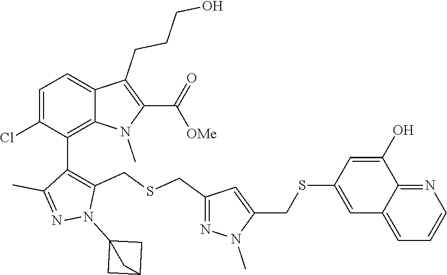

Intermediate 91 was synthesized from Intermediate 90 following a procedure for the preparation of Intermediate 22. MS (LCMS) 757.6 [M+H]⁺.

### Intermediate 92

### Methyl (Z)-2¹-(bicyclo[1.1.1]pentan-1-yl)-1⁶-chloro-1¹,2³,6¹-trimethyl-1¹H,2¹H,6¹H-10-oxa-4,8-dithia-9(6,8)-quinolina-1(7,3)-indola-2(4,5),6(3,5)-dipyrazolacyclotridecaphane-1²-carboxylate

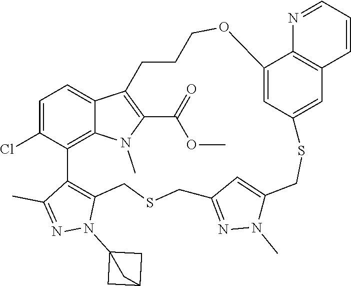

Intermediate 92 was synthesized from Intermediate 91 following a procedure for the preparation of Intermediate 23. MS (LCMS) 739.6 [M+H]⁺.

### Intermediate 93A

### (Rₐ)-(+)-Methyl (Z)-2¹-(bicyclo[1.1.1]pentan-1-yl)-1⁶-chloro-1¹,2³,6¹-trimethyl-9¹,9²,9³,9⁴-tetrahydro-1¹H,2¹H,6¹H-10-oxa-4,8-dithia-9(6,8)-quinolina-1(7,3)-indola-2(4,5),6(3,5)-dipyrazolacyclotridecaphane-1²-carboxylate

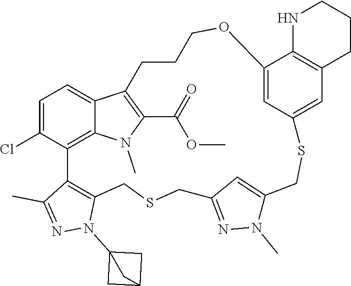

### Intermediate 93B

### (Sₐ)-(−)-Methyl (Z)-2¹-(bicyclo[1.1.1]pentan-1-yl)-1⁶-chloro-1¹,2³,6¹-trimethyl-9¹,9²,9³,9⁴-tetrahydro-1¹H,2¹H,6¹H-10-oxa-4,8-dithia-9(6,8)-quinolina-1(7,3)-indola-2(4,5),6(3,5)-dipyrazolacyclotridecaphane-1²-carboxylate

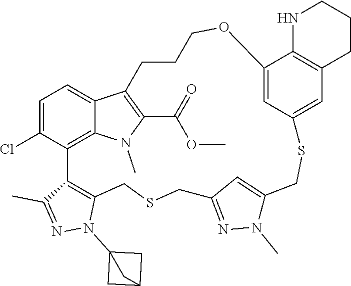

Intermediates 93A and 93B were synthesized from Intermediate 92 following a procedure for the preparation of Intermediates 24A and 24B to give racemic methyl (Z)-2¹-(bicyclo[1.1.1]pentan-1-yl)-1⁶-chloro-1¹,2³,6¹-trimethyl-9¹,9²,9³,9⁴-tetrahydro-1¹H,2¹H,6¹H-10-oxa-4,8-dithia-9(6,8)-quinolina-1(7,3)-indola-2(4,5),6(3,5)-dipyrazolacyclotridecaphane-1²-carboxylate (200 mg, 22%). The atropisomers were separated by chiral SFC chromatography (Lux Cellulose-2 (30×250 mm) column, 40% MeOH) to give peak 1 (Intermediate 93A, 70 mg) and peak 2 (Intermediate 93B, 72 mg). Intermediate 93A: off-white solid; 99.9% chiral purity; MS (LCMS) 743.9 [M+H]⁺. Intermediate 93B: off-white solid; 98.9% chiral purity; MS (LCMS) 743.9 [M+H]⁺. The absolute stereochemistry of Intermediate 93A and Intermediate 93B was arbitrarily assigned.

### Example 9A

### (Rₐ)-(+)-(Z)-2¹-(Bicyclo [1.1.1]pentan-1-yl)-1⁶-chloro-1¹,2³,6¹-trimethyl-9¹,9²,9³,9⁴-tetrahydro-1¹H,2¹H,6¹H-10-oxa-4,8-dithia-9(6,8)-quinolina-1(7,3)-indola-2(4,5),6(3,5)-dipyrazolacyclotridecaphane-1²-carboxylic acid

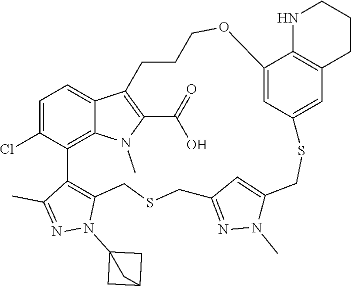

Example 9A was synthesized from Intermediate 93A following a procedure for the preparation of Example 1. Example 9A: (22 mg, 32%), yellow solid; 99.9% chiral purity; ¹H NMR (400 MHz, DMSO-d6) δ 13.26 (br s, 1H), 7.70 (d, J=8.8 Hz, 1H), 7.10 (d, J=8.4 Hz, 1H), 6.64 (s, 1H), 5.84 (s, 1H), 5.33 (br s, 1H), 4.76 (s, 1H), 3.90-3.85 (m, 1H), 3.70-3.60 (m, 2H), 3.60-3.50 (m, 7H), 3.33-3.20 (m, 6H), 3.10-2.95 (m, 1H), 2.70-2.50 (m, 4H), 2.35-2.25 (m, 6H), 2.15-2.05 (m, 1H), 2.04-1.95 (m, 1H), 1.90 (s, 3H), 1.85-1.75 (m, 2H); MS (LCMS) 729.3 [M+H]⁺.

### Example 9B

### (Sₐ)-(−)-(Z)-2¹-(Bicyclo[1.1.1]pentan-1-yl)-1⁶-chloro-1¹,2³,6¹-trimethyl-9¹,9²,9³,9⁴-tetrahydro-1¹H,2¹H,6¹H-10-oxa-4,8-dithia-9(6,8)-quinolina-1(7,3)-indola-2(4,5),6(3,5)-dipyrazolacyclotridecaphane-1²-carboxylic acid

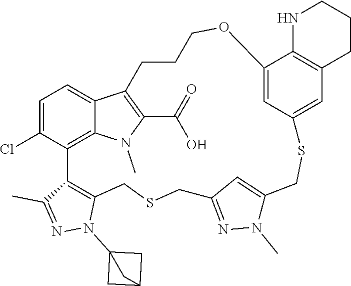

Example 9B was synthesized from Intermediate 93B following a procedure for the preparation of Example 1. Example 9B: (45 mg, 65%), yellow solid; 99.8% chiral purity; ¹H NMR (400 MHz, DMSO-d6) δ 13.30 (br s, 1H), 7.65-7.60 (m, 1H), 7.04 (d, J=8.4 Hz, 1H), 6.61 (s, 1H), 5.90 (s, 1H), 5.29 (br s, 1H), 4.75 (s, 1H), 3.95-3.85 (m, 1H), 3.70-3.60 (m, 2H), 3.54 (s, 7H), 3.33-3.20 (m, 5H), 3.10-2.95 (m, 2H), 2.70-2.50 (m, 4H), 2.35-2.25 (m, 6H), 2.15-2.05 (m, 1H), 2.04-1.95 (m, 1H), 1.90 (s, 3H), 1.85-1.75 (m, 2H); MS (LCMS) 729.3 [M+H]+. The absolute stereochemistry of Example 9A and Example 9B was arbitrarily assigned.

### Intermediate 94

### Methyl 7-(1-(bicyclo[1.1.1]pentan-1-yl)-5-(((4-methoxybenzyl)oxy)methyl)-3-methyl-1H-pyrazol-4-yl)-6-fluoro-3-(3-methoxy-3-oxopropyl)-1H-indole-2-carboxylate

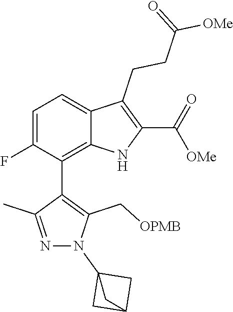

Intermediate 94 was prepared from Intermediate 85 and Intermediate 61 following a procedure for the preparation of Intermediate 17. MS (LCMS) 576.5 [M+H]⁺.

### Intermediate 95

### Methyl 7-(1-(bicyclo[1.1.1]pentan-1-yl)-5-(((4-methoxybenzyl)oxy)methyl)-3-methyl-1H-pyrazol-4-yl)-6-fluoro-3-(3-methoxy-3-oxopropyl)-1-methyl-1H-indole-2-carboxylate

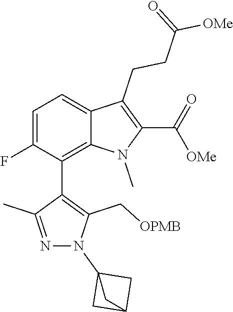

To a stirred solution of Intermediate 94 (6.50 g, 11.3 mmol) in dry DMF (65 mL) were added Cs₂CO₃ (5.53 g, 17.0 mmol) and MeI (1.41 mL, 22.6 mmol). The reaction was stirred at rt for 1 h. The reaction was quenched with water (300 mL) and extracted with EtOAc (3×300 mL). The combined organic layers were washed with brine (500 mL), dried (Na₂SO₄), filtered and the solvent evaporated. The residue was purified by flash chromatography (SiO₂, 60% EtOAc/Pet. ether) to afford Intermediate 95 (5.5 g, 83%) as an oil. MS (LCMS) 590.9 [M+H]⁺.

### Intermediate 96

### Methyl 7-(1-(bicyclo[1.1.1]pentan-1-yl)-5-(((4-methoxybenzyl)oxy)methyl)-3-methyl-1H-pyrazol-4-yl)-6-fluoro-3-(3-hydroxypropyl)-1-methyl-1H-indole-2-carboxylate

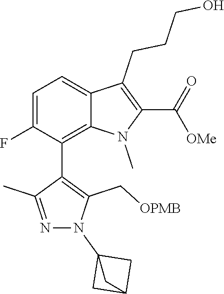

To a suspension of Intermediate 95 (5.30 g, 9.00 mmol) in dry THF (53 mL) was added 1.0 M BH₃THF in THF (53.98 mL, 53.98 mmol) dropwise at 0° C. The ice bath was removed, and the reaction was stirred at rt for 6 h. The reaction was quenched with MeOH (54 mL) and 6 M HCl (54 mL) at 0° C. and then stirred for 30 min at 0° C. The ice bath was removed the mixture was stirred at rt for 20 min. The mixture was diluted with water (100 mL) and extracted with 10% MeOH in DCM (2×100 ml). The organic layer was dried (Na₂SO₄), filtered and evaporated to afford Intermediate 96 (5 g, 98%) as a brown solid. MS (LCMS) 562.9 [M+H]⁺.

### Intermediate 97

### Methyl 3-(3-acetoxypropyl)-7-(1-(bicyclo[1.1.1]pentan-1-yl)-5-(((4-methoxybenzyl)oxy)methyl)-3-methyl-1H-pyrazol-4-yl)-6-fluoro-1-methyl-1H-indole-2-carboxylate

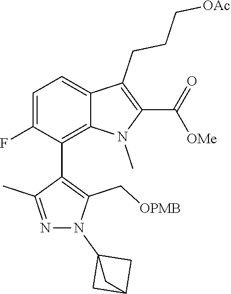

To a stirred solution of Intermediate 96 (5.00 g, 8.91 mmol) in DCM (50 mL) were added TEA (2.62 mL, 18.7 mmol), DMAP (108 mg, 0.89 mmol) and Ac₂O (1.36 mL, 14.4 mmol) at 0° C. The ice bath was removed, and the reaction was stirred at rt for 1 h. Upon completion by TLC, the reaction was quenched with water (50 mL) and extracted with DCM (2×50 mL). The combined organic layers were dried (Na₂SO₄), filtered and evaporated. The residue was purified by flash chromatography (SiO₂, 40% EtOAc/Pet. ether) to afford Intermediate 97 (4 g, 75%) as an oil. MS (LCMS) 604.9 [M+H]⁺.

### Intermediate 98

### Methyl 3-(3-acetoxypropyl)-7-(1-(bicyclo[1.1.1]pentan-1-yl)-5-(hydroxymethyl)-3-methyl-1H-pyrazol-4-yl)-6-fluoro-1-methyl-1H-indole-2-carboxylate

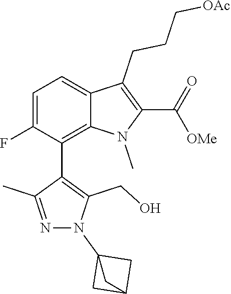 [SMILES: COC(=O)c1c(CCCOC(C)=O)c2ccc(F)c(-c3c(C)nn(C45CC(C4)C5)c3CO)c2n1C]

Intermediate 98 was prepared from Intermediate 97 following a procedure for the preparation of Intermediate 18. MS (LCMS) 484.4 [M+H]⁺.

### Intermediate 99

### Methyl 3-(3-acetoxypropyl)-7-(1-(bicyclo[1.1.1]pentan-1-yl)-5-(chloromethyl)-3-methyl-1H-pyrazol-4-yl)-6-fluoro-1-methyl-1H-indole-2-carboxylate

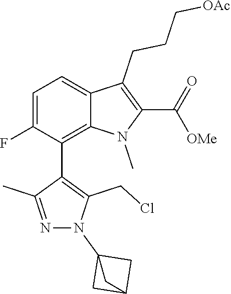 [SMILES: COC(=O)c1c(CCCOC(C)=O)c2ccc(F)c(-c3c(C)nn(C45CC(C4)C5)c3CCl)c2n1C]

Intermediate 99 was prepared from Intermediate 98 following a procedure for the preparation of Intermediate 19. MS (LCMS) 502.8 [M+H]⁺.

### Intermediate 100

### Methyl 3-(3-acetoxypropyl)-7-(1-(bicyclo[1.1.1]pentan-1-yl)-5-(iodomethyl)-3-methyl-1H-pyrazol-4-yl)-6-fluoro-1-methyl-1H-indole-2-carboxylate

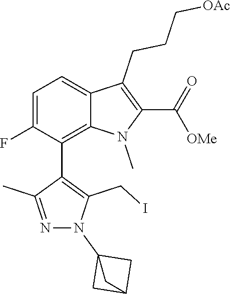 [SMILES: COC(=O)c1c(CCCOC(C)=O)c2ccc(F)c(-c3c(C)nn(C45CC(C4)C5)c3CI)c2n1C]

Intermediate 100 was prepared from Intermediate 99 following a procedure for the preparation of Intermediate 20. MS (LCMS) 594.4 [M+H]⁺.

### Intermediate 101

### Methyl 7-(1-(bicyclo[1.1.1]pentan-1-yl)-5-((((5-(((8-((4-methoxybenzyl)oxy)quinolin-6-yl)thio)methyl)-1-methyl-1H-pyrazol-3-yl)methyl)thio)methyl)-3-methyl-1H-pyrazol-4-yl)-6-fluoro-3-(3-hydroxypropyl)-1-methyl-1H-indole-2-carboxylate

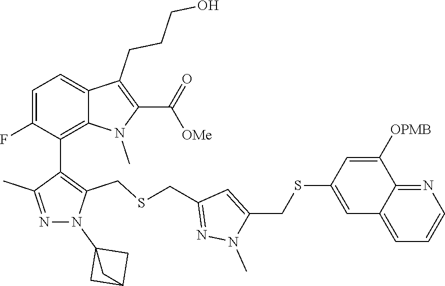

Intermediate 101 was synthesized from Intermediate 100 and Intermediate 11 following a procedure for the preparation of Intermediate 21. MS (LCMS) 861.7 [M+H]⁺.

### Intermediate 102

### Methyl 7-(1-(bicyclo[1.1.1]pentan-1-yl)-5-((((5-(((8-hydroxyquinolin-6-yl)thio)methyl)-1-methyl-1H-pyrazol-3-yl)methyl)thio)methyl)-3-methyl-1H-pyrazol-4-yl)-6-fluoro-3-(3-hydroxypropyl)-1-methyl-1H-indole-2-carboxylate

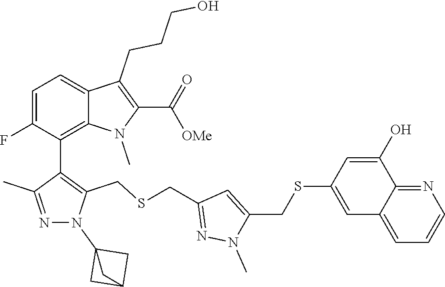 [SMILES: COC(=O)c1c(CCCO)c2ccc(F)c(-c3c(C)nn(C45CC(C4)C5)c3CSCc3cc(CSc4cc(O)c5ncccc5c4)n(C)n3)c2n1C]

Intermediate 102 was synthesized from Intermediate 101 following a procedure for the preparation of Intermediate 22. MS (LCMS) 741.6 [M+H]⁺.

### Intermediate 103

### Methyl (Z)-2¹-(bicyclo[1.1.1]pentan-1-yl)-1⁶-fluoro-1¹,2³,6¹-trimethyl-1¹H,2¹H,6¹H-10-oxa-4,8-dithia-9(6,8)-quinolina-1(7,3)-indola-2(4,5),6(3 ,5)-dipyrazolacyclotridecaphane-1²-carboxylate

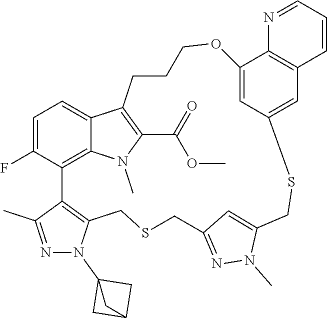 [SMILES: COC(=O)c1c2c3ccc(F)c(c3n1C)-c1c(C)nn(C34CC(C3)C4)c1CSCc1cc(n(C)n1)CSc1cc(c3ncccc3c1)OCCC2]

Intermediate 103 was synthesized from Intermediate 102 following a procedure for the preparation of Intermediate 23. MS (LCMS) 723.4 [M+H]⁺.

### Intermediate 104A

### (Rₐ)-(+)-Methyl (Z)-2¹-(bicyclo[1.1.1]pentan-1-yl)-16-fluoro-1¹,2³,6¹-trimethyl-9¹,9²,9³,9⁴-tetrahydro-1¹H,2¹H,6¹H-10-oxa-4,8-dithia-9(6,8)-quinolina-1(7,3)-indola-2(4,5),6(3,5)-dipyrazolacyclotridecaphane-1²-carboxylate

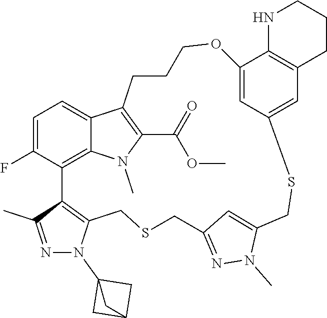

### Intermediate 104B

### (Sₐ)-(−)-Methyl (Z)-2¹-(bicyclo[1.1.1]pentan-1-yl)-1⁶-fluoro-1¹,2³,6¹-trimethyl-9¹,9²,9³,9⁴-tetrahydro-1¹H,2¹H,6¹H-10-oxa-4,8-dithia-9(6,8)-quinolina-1(7,3)-indola-2(4,5),6(3,5)-dipyrazolacyclotridecaphane-1²-carboxylate

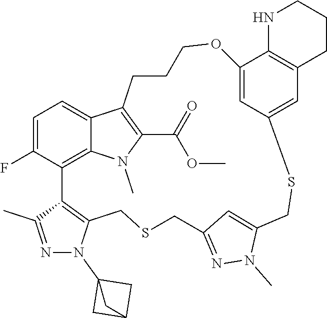

Intermediates 104A and 104B were synthesized from Intermediate 103 following a procedure for the preparation of Intermediates 24A and 24B to give racemic methyl (Z)-2¹-(bicyclo[1.1.1]pentan-1-yl)-1⁶-fluoro-1¹,2³,6¹-trimethyl-9¹,9²,9³,9⁴-tetrahydro-1¹H,2¹H,6¹H-10-oxa-4,8-dithia-9(6,8)-quinolina-1(7,3)-indola-2(4,5),6(3,5)-dipyrazolacyclotridecaphane-1²-carboxylate (250 mg, 30%). The atropisomers were separated by chiral SFC chromatography (Chiralcel OX-3 (30×250 mm) column, 30% (0.2% 7M Methanolic NH₃ in CH₃CN:MeOH; 1:1)) to give peak 1 (Intermediate 104A, 95 mg) and peak 2 (Intermediate 104B, 100 mg). Intermediate 104A: off-white solid; 99.6% chiral purity; MS (LCMS) 727.6 [M+H]⁺. Intermediate 104B: off-white solid; 99.2% chiral purity; MS (LCMS) 727.6 [M+H]⁺. The absolute stereochemistry of Intermediate 104A and Intermediate 104B was arbitrarily assigned.

### Example 10A

### (Rₐ)-(+)-(Z)-2¹-(Bicyclo[1.1.1]pentan-1-yl)-1⁶-fluoro-1¹,2³,6¹-trimethyl-9¹,9²,9³,9⁴-tetrahydro-1¹H,2¹H,6¹H-10-oxa-4,8-dithia-9(6,8)-quinolina-1(7,3)-indola-2(4,5),6(3,5)-dipyrazolacyclotridecaphane-1²-carboxylic acid

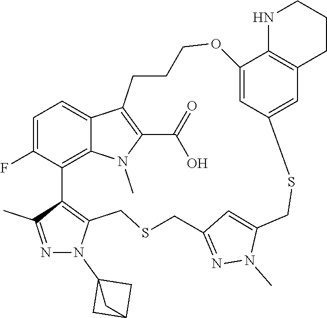

Example 10A was synthesized from Intermediate 104A following a procedure for the preparation of Example 1. Example 10A: (76 mg, 86%), yellow solid; 99.8% chiral purity; ¹H NMR (400 MHz, DMSO-d₆) δ 13.20 (brs, 1H), 7.57 (brs, 1H), 6.80 (t, J=9.2 Hz, 1H), 6.54 (s, 1H), 6.05 (s, 1H), 5.21 (brs, 1H), 4.77 (s, 1H), 3.90 (d, J=14.8 Hz, 1H), 3.69 (dd, J=14.8 Hz, 5.6 Hz, 2H), 3.60-3.40 (m, 8H), 3.35-3.15 (m, 4H), 3.10-2.90 (m 2H), 2.65-2.58 (m, 4H), 2.49-2.30 (m, 6H), 2.27-2.07 (m, 2H), 1.92 (s, 3H), 1.78-1.76 (m, 2H); MS (LCMS) 713.6 [M+H]⁺.

### Example 10B

### (Sₐ)-(−)-(Z)-2¹-(Bicyclo[1.1.1]pentan-1-yl)-1⁶-fluoro-1¹,2³,6¹-trimethyl-9¹,9²,9³,9⁴-tetrahydro-1¹H,2¹H,6¹H-10-oxa-4,8-dithia-9(6,8)-quinolina-1(7,3)-indola-2(4,5),6(3,5)-dipyrazolacyclotridecaphane-1²-carboxylic acid

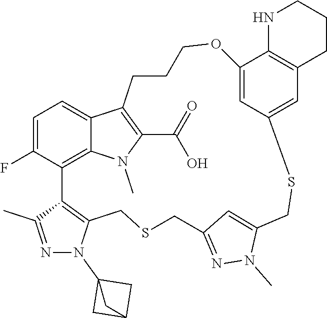

Example 10B was synthesized from Intermediate 104B following a procedure for the preparation of Example 1. Example 10B: (71 mg, 67%), yellow solid; 94.5% chiral purity; ¹H NMR (400 MHz, DMSO-d6) δ 13.20 (brs, 1H), 7.72-7.69 (m, 1H), 6.90 (t, J=9.2 Hz, 1H), 6.59 (s, 1H), 5.96 (s, 1H), 5.27 (brs, 1H), 4.77 (s, 1H), 3.88 (d, J=14.8 Hz, 1H), 3.69 (dd, J=14.8 Hz, 5.6 Hz, 2H), 3.60-3.50 (m, 7H), 3.32-3.20 (m, 5H) 3.10-2.90 (m, 2H), 2.65-2.58 (m, 4H), 2.33-2.30 (m, 6H), 2.27-2.03 (m, 2H), 1.93 (s, 3H), 1.89-1.79 (m, 2H); MS (LCMS) 713.6 [M+H]⁺. The absolute stereochemistry of Example 10A and Example 10B was arbitrarily assigned.

### Intermediate 105

### Methyl 6-chloro-3-(3-hydroxypropyl)-7-(2-(((4-methoxybenzyl)amino)methyl)-5,6-dihydro-4H-pyrrolo[1,2-b]pyrazol-3-yl)-1-methyl-1H-indole-2-carboxylate

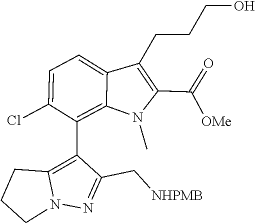

To a stirred solution of Intermediate 38 (4.00 g, 8.75 mmol) in MeOH (50 mL) was added TEA (2.43 mL, 17.5 mmol) and the reaction was stirred for 10 min. The reaction was cooled to 0° C. and PMB-NH₂ (2.90 g, 17.5 mmol) was added. The ice bath was removed, and the reaction was stirred at rt for 12 h. The reaction was cooled to 0° C. and NaBH₄ (660 mg, 17.5 mmol) was added. The reaction was stirred at rt for 2 h. Upon completion by TLC, the reaction was concentrated, and water (100 mL) was added. The mixture was extracted with EtOAc (2×150 mL). The combined organic layers were washed with brine (300 mL), dried (Na₂SO₄), filtered and concentrated. The crude residue was purified by flash chromatography (SiO₂, 5% MeOH:DCM) to afford Intermediate 105 (3.6 g, 56%) as a white solid. MS (LCMS) 537.5 [M+H]⁺.

### Intermediate 106

### Methyl 6-chloro-3-(3-hydroxypropyl)-7-(2-(((4-methoxybenzyl)((5-(((8-((4-methoxybenzyl)oxy)quinolin-6-yl)thio)methyl)-1-methyl-1H-pyrazol-3-yl)methyl)amino)-methyl)-5,6-dihydro-4H-pyrrolo[1,2-b]pyrazol-3-yl)-1-methyl-1H-indole-2-carboxylate

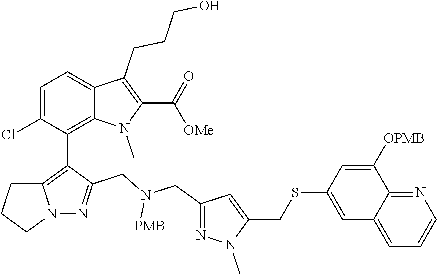

Intermediate 106 was synthesized from Intermediate 105 and Intermediate 10 following a procedure for the preparation of Intermediate 40. MS (LCMS) 940.5 [M+H]⁺.

### Intermediate 107

### Methyl 6-chloro-3-(3-hydroxypropyl)-7-(2-((((5-(((8-hydroxyquinolin-6-yl)thio)methyl)-1-methyl-1H-pyrazol-3-yl)methyl)(4-methoxybenzyl)amino)methyl)-5,6-dihydro-4H-pyrrolo[1,2-b]pyrazol-3-yl)-1-methyl-1H-indole-2-carboxylate

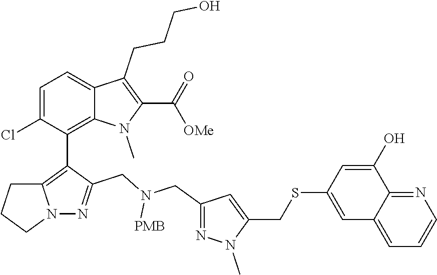

Intermediate 107 was synthesized from Intermediate 106 following a procedure for the preparation of Intermediate 22. MS (LCMS) 820.4 [M+H]⁺.

### Intermediate 108

### Methyl (Z)-1⁶-chloro-4-(4-methoxybenzyl)-1¹,6¹-dimethyl-2⁵,2⁶-dihydro-1¹H,2⁴H,6¹H-10-oxa-8-thia-4-aza-9(6, 8)-quinolina-1(7,3)-indola-2(3,2)-pyrrolo[1,2-b]pyrazola-6(3,5)-pyrazolacyclotridecaphane-1²-carboxylate

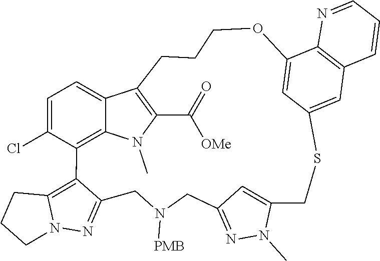

Intermediate 108 was synthesized from Intermediate 107 following a procedure for the preparation of Intermediate 23. MS (LCMS) 802.4 [M+H]⁺.

### Intermediate 109

### Methyl (Z)-1⁶-chloro-1¹,6¹-dimethyl-2⁵,2⁶-dihydro-1¹H,2⁴H,6¹H-10-oxa-8-thia-4-aza-9(6,8)-quinolina-1(7,3)-indola-2(3,2)-pyrrolo[1,2-b]pyrazola-6(3,5)-pyrazolacyclotridecaphane-1²-carboxylate

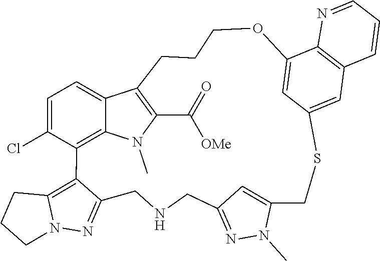 [SMILES: COC(=O)c1c2c3ccc(Cl)c(c3n1C)-c1c(nn3c1CCC3)CNCc1cc(n(C)n1)CSc1cc(c3ncccc3c1)OCCC2]

To a stirred solution of Intermediate 108 (800 mg, 0.998 mmol) in anisole (0.750 mL, 6.99 mmol) was added TFA (2.5 mL). The reaction was stirred at 100° C. for 16 h. After completion, the reaction was diluted with DCM (50 mL) and sat. NaHCO₃ (50 mL). Two reactions were run on equivalent scale. The organic layer was collected, dried (Na₂SO₄), filtered and evaporated to afford Intermediate 109 (1.2 g, 84%) as a white solid. MS (LCMS) 682.5 [M+H]⁺.

### Intermediate 110

### 4-(tert-Butyl) 1²-methyl (Z)-1⁶-chloro-1¹,6¹-dimethyl-2⁵,2⁶-dihydro-1¹H,2⁴H,6¹H-10-oxa-8-thia-4-aza-9(6,8)-quinolina-1(7,3)-indola-2(3,2)-pyrrolo[1,2-b]pyrazola-6(3,5)-pyrazolacyclotridecaphane-1²,4-dicarboxylate

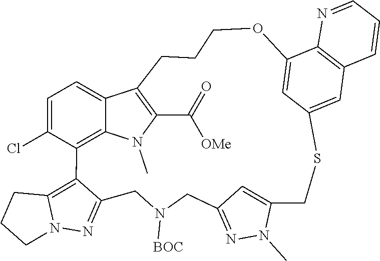 [SMILES: COC(=O)c1c2c3ccc(Cl)c(c3n1C)-c1c(nn3c1CCC3)CN(C(=O)OC(C)(C)C)Cc1cc(n(C)n1)CSc1cc(c3ncccc3c1)OCCC2]

To a stirred solution of Intermediate 109 (1.30 g, 1.91 mmol) in DCM (15 mL) at 0° C. were added TEA (0.50 mL, 3.8 mmol) and di-tert-butyl dicarbonate (0.60 mL, 2.9 mmol). The reaction was stirred at rt for 3 h. Upon completion by LCMS, the reaction was diluted with DCM (50 mL) and water (50 mL). The organic layer was separated, dried (Na₂SO₄), filtered and evaporated. The crude residue was purified by flash chromatography (SiO₂, 5% MeOH:DCM) to afford Intermediate 110 (1.2 g, 80%) as a white solid. MS (LCMS) 782.6 [M+H]⁺.

### Intermediate 111

### 4-(tert-Butyl) 1²-methyl (Z)-1⁶-chloro-1¹,6¹-dimethyl-2⁵,2⁶,9¹,9²,9³,9⁴-hexahydro-1¹H,2⁴H,6¹H-10-oxa-8-thia-4-aza-9(6,8)-quinolina-1(7,3)-indola-2(3,2)-pyrrolo[1,2-b]pyrazola-6(3,5)-pyrazolacyclotridecaphane-1²,4-dicarboxylate

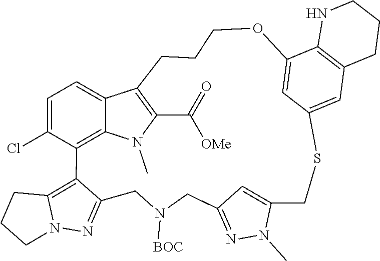 [SMILES: COC(=O)c1c2c3ccc(Cl)c(c3n1C)-c1c(nn3c1CCC3)CN(C(=O)OC(C)(C)C)Cc1cc(n(C)n1)CSc1cc3c(c(c1)OCCC2)NCCC3]

Intermediate 111 was synthesized from Intermediate 110 following a procedure for the preparation of Intermediates 24A and 24B. MS (LCMS) 786.5 [M+H]⁺.

### Intermediate 112A

### (Rₐ)-(+)-Methyl (Z)-1⁶-chloro-1¹,6¹-dimethyl-2⁵,2⁶,9¹,9²,9³,9⁴-hexahydro-1¹H,2⁴H,6¹H-10-oxa-8-thia-4-aza-9(6,8)-quinolina-1(7,3)-indola-2(3,2)-pyrrolo[1,2-b]pyrazola-6(3,5)-pyrazolacyclotridecaphane-1²-carboxylate

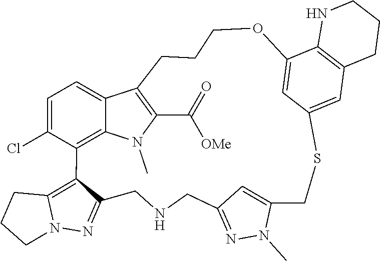

### Intermediate 112B

### (Sₐ)-(−)-Methyl (Z)-1⁶-chloro-1¹,6¹-dimethyl-2⁵,2⁶,9¹,9²,9³,9⁴-hexahydro-1¹H,2⁴H,6¹H-10-oxa-8-thia-4-aza-9(6,8)-quinolina-1(7,3)-indola-2(3,2)-pyrrolo[1,2-b]pyrazola-6(3,5)-pyrazolacyclotridecaphane-1²-carboxylate

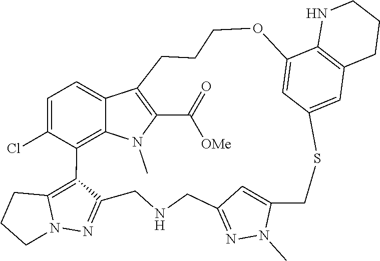

To a stirred solution of Intermediate 111 (700 mg, 0.890 mmol) in DCM (8 mL) at 0° C. was added 4 M HCl in 1,4-dioxane (4.0 mL). The ice bath was removed, and the reaction was stirred at rt for 2 h. The reaction was diluted with DCM (50 mL) and sat. NaHCO₃ (50 mL). The organic layer was separated, dried (Na₂SO₄), filtered and evaporated to provide racemic methyl (Z)-1⁶-chloro-1¹,6¹-dimethyl-2⁵,2⁶,9¹,9²,9³,9⁴-hexahydro-1¹H,2⁴H,6¹H-10-oxa-8-thia-4-aza-9(6,8)-quinolina-1(7,3)-indola-2(3,2)-pyrrolo[1,2-b]pyrazola-6(3,5)-pyrazolacyclotridecaphane-1²-carboxylate (210 mg, 21%) as a white solid. MS (LCMS) 686.6 [M+H]⁺. The atropisomers were separated by chiral SFC chromatography (Chiralcel-OX-3 (30×250 mm) column, 50% (0.2% 7M NH₃ in MeOH, CH₃CN:MeOH; 1:1)) to give peak 1 (Intermediate 112A, 40 mg) and peak 2 (Intermediate 112B, 100 mg). Intermediate 112A: off-white solid; 98.1% chiral purity; MS (LCMS) 686.7 [M+H]⁺. Intermediate 112B: off-white solid; 99.6% chiral purity; MS (LCMS) 686.4 [M+H]⁺. The absolute stereochemistry of Intermediate 112A and Intermediate 112B was arbitrarily assigned.

### Example 11A

### (Rₐ)-(+)-(Z)-1⁶-Chloro-1¹,6¹-dimethyl-2⁵,2⁶,9¹,9²,9³,9⁴-hexahydro-1¹H,2⁴H,6¹H-10-oxa-8-thia-4-aza-9(6,8)-quinolina-1(7,3)-indola-2(3,2)-pyrrolo[1,2-b]pyrazola-6(3,5)-pyrazolacyclotridecaphane-1²-carboxylic acid

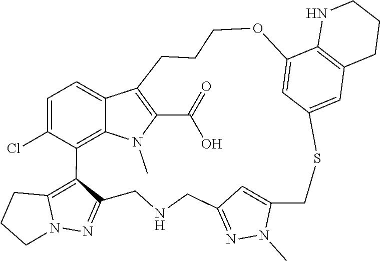

Example 11A was synthesized from Intermediate 112A following a procedure for the preparation of Example 1. Example 11A: (56 mg, 78%), white solid; 98.8% chiral purity; ¹H NMR (400 MHz, DMSO-d₆) δ 7.83 (d, J=8.4 Hz, 1H), 7.18 (d, J=8.8 Hz, 1H), 6.52 (s, 1H), 6.22 (s, 1H), 5.20-5.05 (m, 2H), 4.20 (t, J=7.4 Hz, 2H), 4.00-3.90 (m, 2H), 3.70-3.40 (m, 13H), 3.30-3.00 (m, 4H), 2.85-2.50 (m, 6H), 2.20-2.00 (m, 2H), 1.80-1.65 (m, 2H); MS (LCMS) 672.4 [M+H]⁺. The absolute stereochemistry of Example 11A was arbitrarily assigned.

### Intermediate 113

### Methyl 3-(3-acetoxypropyl)-6-chloro-7-(2-(((4-methoxybenzyl)amino)methyl)-4,5,6,7-tetrahydropyrazolo[1,5-a]pyridin-3-yl)-1-methyl-1H-indole-2-carboxylate

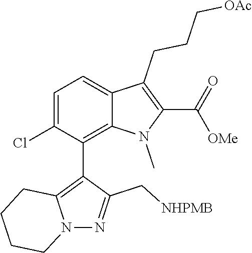

To a stirred solution of Intermediate 44 (2.50 g, 5.30 mmol) in MeOH (50 mL) at 0° C. were added (4-methoxyphenyl)methanamine (872 mg, 6.36 mmol) and Et₃N (2.20 mL, 15.9 mmol). The ice bath was removed, and the reaction was stirred at rt for 14 h. The reaction was cooled to 0° C. and NaBH₄ (294 mg, 7.96 mmol) was added. The ice bath was removed, and the reaction was stirred for 2 h at rt. Upon completion, the reaction was diluted with DCM (100 mL) and water (100 mL). The organic layer was separated, dried (Na₂SO₄), filtered and evaporated. The reaction was repeated on an identical scale. The combined residues were purified by flash chromatography (SiO₂, 5% MeOH:DCM) to afford Intermediate 113 (3.54 g, 33%) as an oil. MS (LCMS) 594.4 [M+H]⁺.

### Intermediate 114

### Methyl 3-(3-acetoxypropyl)-6-chloro-7-(2-(((4-methoxybenzyl)((5-(((8-((4-methoxybenzyl)oxy)quinolin-6-yl)thio)methyl)-1-methyl-1H-pyrazol 1)methyl)amino)methyl)-4,5,6,7-tetrahydropyrazolo[1,5-a]pyridin-3-yl)-1-methyl-1H-indole-2-carboxylate

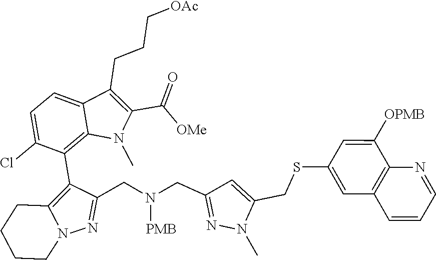

To a stirred solution of Intermediate 113 (1.00 g, 1.69 mmol) in DMF (20 mL) at 0° C. were added K₂CO₃ (465 mg, 3.38 mmol) and Intermediate 10 (1.11 g, 2.53 mmol). The ice bath was removed, and the reaction was stirred at rt for 16 h. The reaction was diluted with water (50 mL) and extracted with EtOAc (2×50 mL). The combined organic layers were dried (Na₂SO₄), filtered and evaporated. The reaction was repeated on an identical scale. The combined residues were purified by flash chromatography (SiO₂, 5% MeOH:DCM) to afford Intermediate 114 (2.5 g, 74%) as a white solid. MS (LCMS) 996.4 [M+H]⁺.

### Intermediate 115

### Methyl 6-chloro-3-(3-hydroxypropyl)-7-(2-(((4-methoxybenzyl)((5-(((8-((4-methoxybenzyl)oxy)quinolin-6-yl)thio)methyl)-1-methyl-1H-pyrazol-3-1)methyl)amino)methyl)-4,5,6,7-tetrahydropyrazolo[1,5-a]pyridin-3-yl)-1-methyl-1H-indole-2-carboxylate

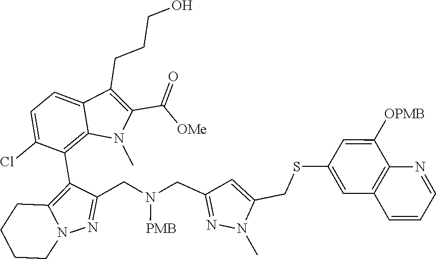

To a stirred solution of Intermediate 114 (1.25 g, 1.26 mmol) in MeOH (15 mL) was added K₂CO₃ (348 mg 2.52 mmol) at 0° C. The ice bath was removed, and the reaction was stirred at rt for 2 h. The reaction was diluted with water (50 mL) and extracted with DCM (100 mL). The reaction was repeated on an identical scale. The combined organic layers were separated, dried (Na₂SO₄), filtered and evaporated to afford Intermediate 115 (2.3 g, 96%) as an off-white solid. MS (LCMS) 954.5 [M+H]⁺.

### Intermediate 116

### Methyl 6-chloro-3-(3-hydroxypropyl)-7-(2-((((5-(((8-hydroxyquinolin-6-yl)thio)methyl)-1-methyl-1H-pyrazol-3-yl)methyl)(4-methoxybenzyl)amino)methyl)-4,5,6,7-tetrahydropyrazolo[1,5-a]pyridin-3-yl)-1-methyl-1H-indole-2-carboxylate

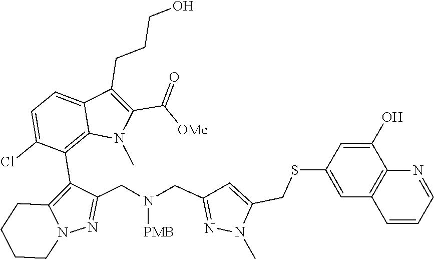

To a stirred solution of Intermediate 115 (1.20 g, 1.26 mmol) in DCM (15 mL) at 0° C. was added TFA (1.0 mL). The ice bath was removed, and the reaction was stirred rt for 3 h. Upon completion, the reaction was diluted with DCM (50 mL) and sat. NaHCO₃ (50 mL). The organic layer was separated, dried (Na₂SO₄), filtered and evaporated. The reaction was repeated on an identical scale. The combined residues were purified by flash chromatography (SiO₂, 5% MeOH:DCM) to afford Intermediate 116 (950 mg, 52%) as a white solid. MS (LCMS) 834.6 [M+H]⁺.

### Intermediate 117

### Methyl (Z)-1⁶-chloro-4-(4-methoxybenzyl)-1¹,6¹-dimethyl-2⁴,2⁵,2⁶,2⁷-tetrahydro-1¹H,6 ¹H-10-oxa-8-thia-4-aza-9(6,8)-quinolina-2(3 ,2)-pyrazolo[1,5-a]pyridina-1(7,3)-indola-6(3,5)-pyrazolacyclotridecaphane-1²-carboxylate

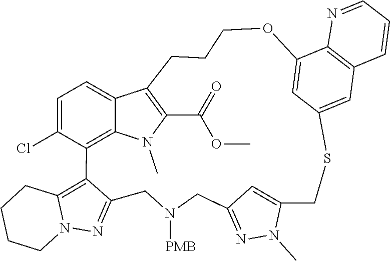

Intermediate 117 was synthesized from Intermediate 116 following a procedure for the preparation of Intermediate 23. MS (LCMS) 816.4 [M+H]⁺.

### Intermediate 118

### Methyl (Z)-1⁶-chloro-1¹,6¹-dimethyl-2⁴,2⁵,2⁶,2⁷-tetrahydro-1¹H,6¹H-10-oxa-8-thia-4-aza-9(6,8)-quinolina-2(3,2)-pyrazolo[1,5-a]pyridina-1(7,3)-indola-6(3,5)-pyrazolacyclotridecaphane-1²-carboxylate

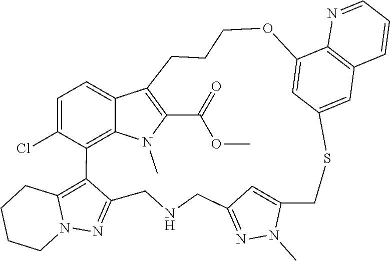 [SMILES: COC(=O)c1c2c3ccc(Cl)c(c3n1C)-c1c(nn3c1CCCC3)CNCc1cc(n(C)n1)CSc1cc(c3ncccc3c1)OCCC2]

To a stirred solution of Intermediate 117 (400 mg, 0.490 mmol) in anisole (2.5 mL) was added TFA (2.5 mL). The reaction was stirred at 80° C. for 16 h. Upon completion, the reaction was diluted with DCM (50 mL) and sat. NaHCO₃ (50 mL). The organic layer was separated, dried (Na₂SO₄), filtered and evaporated to afford Intermediate 118 (450 mg) as a white solid. MS (LCMS) 696.6 [M+H]⁺.

### Intermediate 119

### 4-(tert-Butyl) 1²-methyl (Z)-1⁶-chloro-1¹,6¹-dimethyl-2⁴,2⁵,2⁶,2⁷-tetrahydro-1¹H,6¹H-10-oxa-8-thia-4-aza-9(6,8)-quinolina-2(3,2)-pyrazolo[1,5-a]pyridina-1(7,3)-indola-6(3,5)-pyrazolacyclotridecaphane-1²,4-dicarboxylate

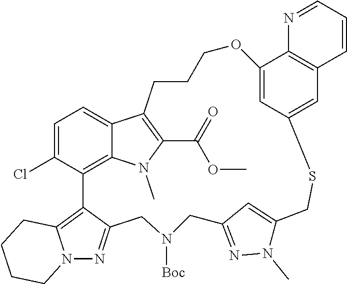 [SMILES: COC(=O)c1c2c3ccc(Cl)c(c3n1C)-c1c(nn3c1CCCC3)CN(C(=O)OC(C)(C)C)Cc1cc(n(C)n1)CSc1cc(c3ncccc3c1)OCCC2]

To a stirred solution of Intermediate 118 (450 mg, 0.647 mmol) in DCM (10 mL) at 0° C. were added TEA (0.450 mL, 3.23 mmol) and di-tert-butyl dicarbonate (0.300 mL, 1.29 mmol). The ice bath was removed, and the reaction was stirred at rt for 3 h. Upon completion, the reaction was diluted with DCM (50 mL) and water (50 mL). The organic layer was separated, dried (Na₂SO₄), filtered and evaporated. The residue was purified by flash chromatography (SiO₂, 5% MeOH:DCM) to afford Intermediate 119 (350 mg, 68% 2-steps) as a white solid. MS (LCMS) 796.9 [M+H]⁺.

### Intermediate 120

### 4-(tert-Butyl) 1²-methyl (Z)-1⁶-chloro-1¹,6¹-dimethyl-2⁴,2⁵,2⁶,9¹,9²,9³,9⁴-octahydro-1¹H,6¹H-10-oxa-8-thia-4-aza-9(6,8)-quinolina-2(3,2)-pyrazolo[1,5-a]pyridina-1(7,3)-indola-6(3,5)-pyrazolacyclotridecaphane-1²,4-dicarboxylate

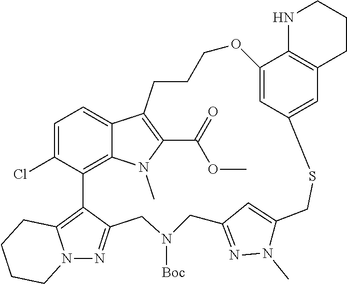 [SMILES: COC(=O)c1c2c3ccc(Cl)c(c3n1C)-c1c(nn3c1CCCC3)CN(C(=O)OC(C)(C)C)Cc1cc(n(C)n1)CSc1cc3c(c(c1)OCCC2)NCCC3]

To a stirred solution of Intermediate 119 (350 mg, 0.440 mmol) in AcOH (4 mL) and MeOH (4 mL) under N₂ was added NaCNBH₃ (277 mg, 4.40 mmol). The reaction was stirred at rt for 3 h. The reaction was concentrated, diluted with DCM (50 mL) and washed with saturated NaHCO₃ (2×50 mL). The organic layer was separated, dried (Na₂SO₄), filtered and evaporated. The residue was purified by flash chromatography (SiO₂, 60% EtOAc/Pet. ether) to afford Intermediate 120 (220 mg, 63%) as a white solid. MS (LCMS) 800.4 [M+H]⁺.

### Intermediate 121A

### (Rₐ)-(+)-Methyl (Z)-1⁶-chloro-1¹,6¹-dimethyl-2⁴,2⁵,2⁶,2⁷,9¹,9²,9³,9⁴-octahydro-1¹H,6¹H-10-oxa-8-thia-4-aza-9(6,8)-quinolina-2(3,2)-pyrazolo[1,5-a]pyridina-1(7,3)-indola-6(3,5)-pyrazolacyclotridecaphane-1²-carboxylate

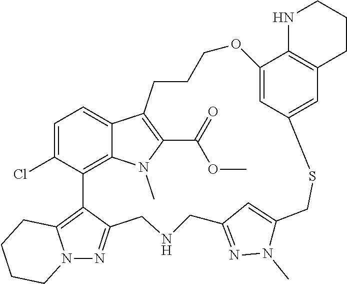 [SMILES: COC(=O)c1c2c3ccc(Cl)c(c3n1C)-c1c(nn3c1CCCC3)CNCc1cc(n(C)n1)CSc1cc3c(c(c1)OCCC2)NCCC3]

### Intermediate 121B

### (Sₐ)-(−)-Methyl (Z)-1⁶-chloro-1¹,6¹-dimethyl-2⁴,2⁵,2⁶,2⁷,9¹,9²,9³,9⁴-octahydro-1¹H,6¹H-10-oxa-8-thia-4-aza-9(6,8)-quinolina-2(3,2)-pyrazolo[1,5-a]pyridina-1(7,3)-indola-6(3,5)-pyrazolacyclotridecaphane-1²-carboxylate

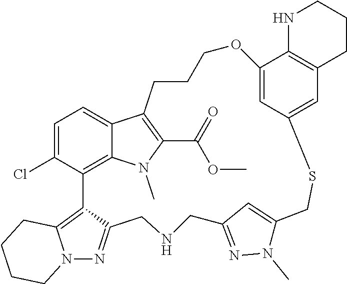

To a stirred solution of Intermediate 120 (220 mg, 0.275 mmol) in DCM (2 mL) at 0° C. was added 4 M HCl in 1,4-dioxane (2 mL). The ice bath was removed, and the reaction was stirred at rt for 2 h. The reaction was diluted with DCM (10 mL) and sat. NaHCO₃ (10 mL). The organic layer was separated, dried (Na₂SO₄), filtered and evaporated to afford racemic methyl (Z)-1⁶-chloro-1¹,6¹-dimethyl-2⁴,2⁵,2⁶,2⁷,9¹,9²,9³,9⁴-octahydro-1¹H,6¹H-10-oxa-8-thia-4-aza-9(6,8)-quinolina-2(3,2)-pyrazolo[1,5-a]pyridina-1(7,3)-indola-6(3,5)-pyrazolacyclotridecaphane-1²-carboxylate (175 mg 90%) as a white solid. MS (LCMS) 700.5 [M+H]⁺. The atropisomers were separated by chiral SFC chromatography (Chiralcel-OX-3 (30×250 mm) column, 50% ((0.2% 7M NH₃ in MeOH, CH₃CN:MeOH; 1:1)) to give peak 1 (Intermediate 121A, 70 mg) and peak 2 (Intermediate 121B, 70 mg). Intermediate 121A: off-white solid; 99.9% chiral purity; MS (LCMS) 700.5 [M+H]⁺. Intermediate 121B: off-white solid; 99.5% chiral purity; MS (LCMS) 700.5 [M+H]⁺. The absolute stereochemistry of Intermediate 121A and Intermediate 121B was arbitrarily assigned.

### Example 12A

### (Rₐ)-(+)-(Z)-1⁶-Chloro-1¹,6¹-dimethyl-2⁴,2⁵,2⁶,2⁷,9¹,9²,9³,9⁴-octahydro-1¹H,6¹H-10-oxa-8-thia-4-aza-9(6,8)-quinolina-2(3,2)-pyrazolo[1,5-a]pyridina-1(7,3)-indola-6(3,5)-pyrazolacyclotridecaphane-1²-carboxylic acid

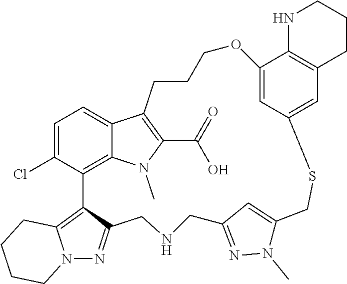

Example 12A was synthesized from Intermediate 121A following a procedure for the preparation of Example 1. Example 12A: (48 mg, 70%), white solid; 98.5% chiral purity; ¹H NMR (400 MHz, DMSO-d₆) δ 13.3 (br s, 1H), 9.42 (brs, 1H), 8.85 (brs, 1H), 7.87 (d, J=8.4 Hz, 1H), 7.20 (d, J=8.8 Hz, 1H), 6.56 (s, 1H), 6.21 (s, 1H), 5.30-5.0 (m, 2H), 4.22-4.17 (m, 2H), 4.05-3.95 (m, 2H), 3.85-3.75 (m, 2H), 3.80-3.40 (m, 10H), 3.30-3.00 (m, 4H), 2.70-2.50 (m, 2H), 2.50-2.30 (m, 2H), 2.25-2.00 (m, 4H), 1.90-1.70 (m, 4H); MS (LCMS) 686.4 [M+H]⁺.

### Example 12B

### (Sₐ )-(−)-(Z)-1⁶-Chloro-1¹,6¹-dimethyl-2⁴,2⁵,2⁶,2⁷,9¹,9²,9³,9⁴-octahydro-1¹H,6¹H-10-oxa-8-thia-4-aza-9(6,8)-quinolina-2(3,2)-pyrazolo[1,5-a]pyridina-1(7,3)-indola-6(3,5)-pyrazolacyclotridecaphane-1²-carboxylic acid

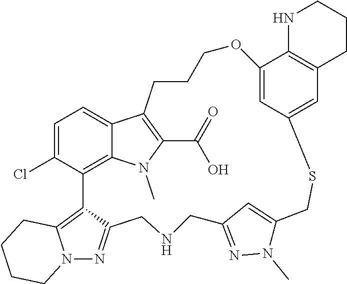

Example 12B was synthesized from Intermediate 121B following a procedure for the preparation of Example 1. Example 12B: (48 mg, 70%), white solid; 93.2% chiral purity; ¹H NMR (400 MHz, DMSO-d₆) δ 7.80 (d, J=8.8 Hz, 1H), 7.18 (d, J=8.4 Hz, 1H), 6.46 (s, 1H), 6.29 (s, 1H), 5.10 (brs, 1H), 4.95 (s, 1H), 4.22-4.17 (m, 2H), 4.02-3.95 (m, 2H), 3.80-3.50 (m, 12H), 3.30-3.00 (m, 4H), 2.60-2.50 (m, 2H), 2.45-2.30 (m, 2H), 2.25-1.90 (m, 4H), 1.80-1.60 (m, 4H); MS (LCMS) 686.4 [M+H]⁺. The absolute stereochemistry of Example 12A and Example 12B was arbitrarily assigned.

### Intermediate 122A

### (Rₐ)-(+)-Methyl (Z)-1⁶-chloro-1¹,6¹,9¹-trimethyl-2⁵,2⁶,9¹,9²,9³,9⁴-hexahydro-1¹H,2⁴H,6¹H-10-oxa-4,8-dithia-9(6,8)-quinolina-1(7,3)-indola-2(3,2)-pyrrolo[1,2-b]pyrazola-6(3,5)-pyrazol acyclotridecaphane-1²-carboxylate

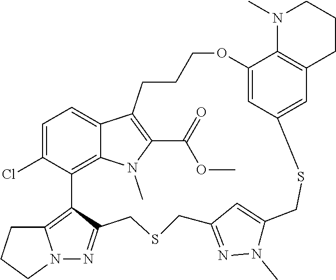

### Intermediate 122B

### (Sₐ)-(−)-Methyl (Z)-1⁶-chloro-1¹,6¹,9¹-trimethyl-2⁵,2⁶,9¹,9²,9³,9⁴-hexahydro-1¹H,2⁴H,6¹H-10-oxa-4,8-dithia-9(6,8)-quinolina-1(7,3)-indola-2(3,2)-pyrrolo[1,2-b]pyrazola-6(3,5)-pyrazol acyclotridecaphane-1²-carboxylate

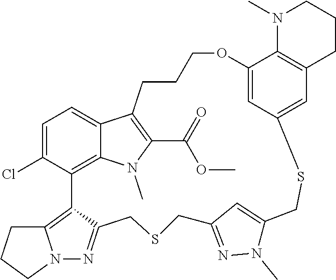

To a stirred solution of racemic methyl (Z)-1⁶-chloro-1¹,6¹-dimethyl-2⁵,2⁶,9¹,9²,9³,9⁴-hexahydro-1¹H,2⁴H,6¹H-10-oxa-4,8-dithia-9(6,8)-quinolina-1(7,3)-indola-2(3,2)-pyrrolo[1,2-b]pyrazola-6(3,5)-pyrazolacyclotridecaphane-1²-carboxylate (1.00 g, 1.42 mmol) in DCE (10 mL), was added 37% formaldehyde (284 mg, 2.85 mmol) and NaOAc (140 mg, 1.71 mmol). The reaction was stirred for 2 h, and NaCNBH₃ (480 mg, 2.28 mmol) was added. The reaction was stirred at rt for 16 h. The reaction was concentrated and partitioned between DCM (50 mL) and sat. NaHCO₃ (50 mL). The organic layer was dried (Na₂SO₄), filtered and evaporated. The crude residue was purified by flash chromatography (SiO₂, EtOAc) to afford racemic methyl (Z)-1⁶-chloro-1¹,6¹,9¹-trimethyl-2⁵,2⁶,9¹,9²,9³,9⁴-hexahydro-1¹H,2⁴H,6¹H-10-oxa-4,8-dithia-9(6,8)-quinolina-1(7,3)-indola-2(3,2)-pyrrolo[1,2-b]pyrazola-6(3,5)-pyrazol acyclotridecaphane-1²-carboxylate (400 mg, 40%) as a white solid. MS (LCMS) 717.7 [M+H]⁺. The atropisomers were separated by chiral SFC chromatography (Chiralcel-OJ-3 (30×250 mm) column, 25% MeOH) to give peak 1 (Intermediate 122A, 170 mg) and peak 2 (Intermediate 122B, 80 mg). Intermediate 122A: off-white solid; 99.1% chiral purity; MS (LCMS) 717.8 [M+H]⁺. Intermediate 122B: off-white solid; 99.5% chiral purity; MS (LCMS) 717.8 [M+H]⁺. The absolute stereochemistry of Intermediate 122A and Intermediate 122B was arbitrarily assigned.

### Example 13A

### (Rₐ)-(+)-(Z)-1⁶-Chloro-1¹,6¹,9¹-trimethyl-2⁵,2⁶,9¹,9²,9³,9⁴-hexahydro-1¹H,2⁴H,6¹H-10-oxa-4,8-dithia-9(6,8)-quinolina-1(7,3)-indola-2(3,2)-pyrrolo[1,2-b]pyrazola-6(3,5)-pyrazolacyclotridecaphane-1²-carboxylic acid

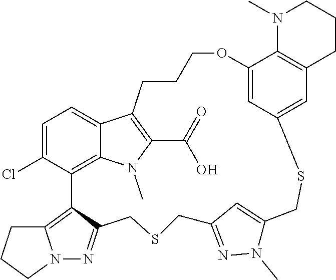

Example 13A was synthesized from Intermediate 122A following a procedure for the preparation of Example 1. Example 13A: (106 mg, 63%), white solid; 97.0% chiral purity; ¹H NMR (400 MHz, DMSO-d₆) δ 13.32 (brs, 1H), 7.92-7.85 (m, 1H), 7.18 (s, 1H), 6.70-6.20 (m, 2H), 4.80 (s, 1H), 4.20-4.05 (m, 4H), 3.75-3.40 (m, 9H), 3.30-3.20 (m, 3H), 3.15-2.50 (m, 13H), 2.30-1.70 (m, 4H); MS (LCMS) 703.4 [M+H]⁺.

### Example 13B

### (Sₐ)-(−)-(Z)-1⁶-Chloro-1¹,6¹,9¹-trimethyl-2⁵,2⁶,9¹,9²,9³,9⁴-hexahydro-1¹H,2⁴H,6¹H-10-oxa-4,8-dithia-9(6,8)-quinolina-1(7,3)-indola-2(3,2)-pyrrolo[1,2-b]pyrazola-6(3 ,5)-pyrazolacyclotridecaphane-1²-carboxylic acid

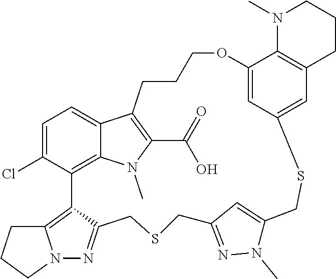

Example 13B was synthesized from Intermediate 122B following a procedure for the preparation of Example 1. Example 13B: (42 mg, 53%), white solid; 98.3% chiral purity; ¹H NMR (400 MHz, DMSO-d6) δ 13.29 (brs, 1H), 7.92-7.85 (m, 1H), 7.19 (brs, 1H), 6.70-6.10 (m, 2H), 4.80 (brs, 1H), 4.20-4.05 (m, 4H), 3.75-3.40 (m, 8H), 3.30-2.50 (m, 17H), 2.30-1.70 (m, 4H); MS (LCMS) 703.6 [M+H]⁺. The absolute stereochemistry of Example 13A and Example 13B was arbitrarily assigned.

### Intermediate 123

### (1-Ethyl-3-methyl-1H-pyrazol-5-yl)methanol

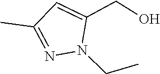

To a stirred solution of 1-ethyl-3-methyl-1H-pyrazole-5-carboxylic acid (35.0 g, 227 mmol) in THF (350 mL) was added 2.4M LiAlH₄ in THF (104 mL, 250 mmol) at 0° C. The reaction was stirred at rt for 2 h. Upon completion by LCMS, the reaction was quenched with cold sat. Na₂SO₄ (200 mL) at 0° C. The reaction was filtered through a Celite pad and washed with EtOAc (2×250 mL). The filtrate was washed with brine (500 mL), dried (Na₂SO₄), filtered and concentrated under reduced pressure to afford Intermediate 123 (30 g, 94%) as an oil. MS (ESI) 141.1 [M+H]⁺.

### Intermediate 124

### (4-Bromo-1-ethyl-3-methyl-1H-pyrazol-5-yl)methanol

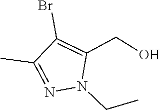

To a stirred solution of Intermediate 123 (30.0 g, 214 mmoL) in DCM (300 mL) was added NBS (40.0 g, 225 mmol) portionwise over 30 min. at 0° C. The ice bath was removed, and the reaction was stirred for 1 h at rt. Upon completion by LCMS, the reaction was quenched with water (500 mL) and diluted with DCM (500 mL). The layers were separated, and the organic layer was washed with brine (500 mL), dried (Na₂SO₄), filtered and concentrated. The crude mixture was triturated with a mixture of n-pentane (300 mL) and diethyl ether (100 mL). The solid was filtered and dried under reduced pressure to afford Intermediate 124 (30 g, 64%) as an off-white solid. MS (ESI) 219.2 [M+H]⁺.

### Intermediate 125

### 4-Bromo-1-ethyl-5-(((4-methoxybenzyl)oxy)methyl)-3-methyl-1H-pyrazole

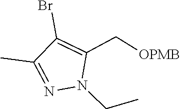

To a stirred solution of Intermediate 124 (30.0 g, 137 mmol) in DMF (300 mL) was added 60% NaH (8.20 g, 342 mmol) at 0° C. The ice bath was removed, and the reaction was stirred at rt for 30 min. 1-(Chloromethyl)-4-methoxybenzene (32.0 g, 205.1 mmol) and KI (4.40 g, 26.5 mmol) were added and the reaction was stirred at rt for 16 h. Upon completion, the reaction was quenched with ice, diluted with water (700 mL) and extracted with EtOAc (3×500 mL). The combined organic layers were washed with water (2×500 mL) and brine (500 mL). The organic layer was dried (Na₂SO₄), filtered and concentrated. The crude material was purified by flash chromatography (SiO₂, 20% EtOAc/Pet. ether) to afford Intermediate 125 (28 g, 60%) as an oil. MS (LCMS) 339.2 [M+H]⁺.

### Intermediate 126

### 1-Ethyl-5-(((4-methoxybenzyl)oxy)methyl)-3-methyl-4-(4,4,5,5-tetramethyl-1,3,2-dioxaborolan-2-yl)-1H-pyrazole

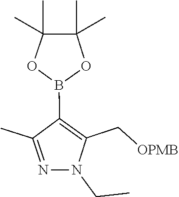

To a stirred solution of Intermediate 125 (28.0 g, 82.8 mmol) in THF (300 mL) was added 1.6 M n-BuLi in hexanes (38.0 mL, 99.4 mmol) at −78° C. The reaction was stirred for 1 h at -78° C. 2-Isopropoxy-4,4,5,5-tetramethyl-1,3,2-dioxaborolane (44.28 g, 238.1 mmol) was added at −78° C. and the reaction was stirred at −78° C. for 1 h. Upon completion, the reaction was quenched with EtOAc (50 mL) and allowed to warm to rt. The solvent was removed, and the crude was diluted with EtOAc (300 mL). The mixture was filtered through a Celite pad which was washed with EtOAc (2×100 mL). The filtrate was evaporated, and the crude product was triturated with n-pentane. The solid was collected by filtration and dried under vacuum to afford Intermediate 126 (17 g, 53%) as an off-white solid. MS (ESI) 387.0 [M+H]⁺.

### Intermediate 127

### Methyl 3-(3-acetoxypropyl)-6-chloro-7-(1-ethyl-5-(((4-methoxybenzyl)oxy)methyl)-3-methyl-1H-pyrazol-4-yl)-1-methyl-1H-indole-2-carboxylate

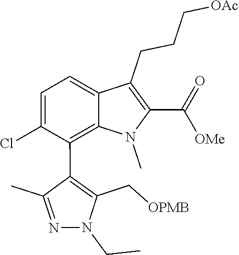

Intermediate 127 was synthesized from Intermediate 4 and Intermediate 126 following a procedure from the preparation of Intermediate 17. MS (LCMS) 582.4 [M+H]⁺.

### Intermediate 128

### Methyl 3-(3-acetoxypropyl)-6-chloro-7-(1-ethyl-5-(hydroxymethyl)-3-methyl-1H-pyrazol-4-yl)-1-methyl-1H-indole-2-carboxylate

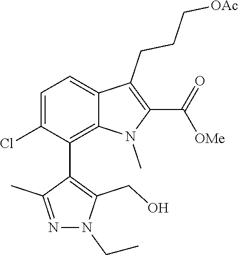 [SMILES: CCn1nc(C)c(-c2c(Cl)ccc3c(CCCOC(C)=O)c(C(=O)OC)n(C)c23)c1CO]

Intermediate 128 was synthesized from Intermediate 127 following a procedure for the preparation of Intermediate 18. MS (LCMS) 462.3 [M+H]⁺.

### Intermediate 129

### Methyl 3-(3-acetoxypropyl)-6-chloro-7-(5-(chloromethyl)-1-ethyl-3-methyl-1H-pyrazol-4-yl)-1-methyl-1H-indole-2-carboxylate

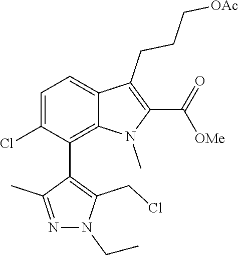 [SMILES: CCn1nc(C)c(-c2c(Cl)ccc3c(CCCOC(C)=O)c(C(=O)OC)n(C)c23)c1CCl]

Intermediate 129 was synthesized from Intermediate 128 following a procedure for the preparation of Intermediate 19. MS (LCMS) 480.3 [M+H]⁺.

### Intermediate 130

### Methyl 3-(3-acetoxypropyl)-6-chloro-7-(1-ethyl-5-(iodomethyl)-3-methyl-1H-pyrazol-4-yl)-1-methyl-1H-indole-2-carboxylate

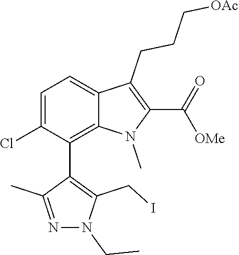 [SMILES: CCn1nc(C)c(-c2c(Cl)ccc3c(CCCOC(C)=O)c(C(=O)OC)n(C)c23)c1CI]

Intermediate 130 was synthesized from Intermediate 129 following a procedure for the preparation of Intermediate 20. MS (LCMS) 572.3 [M+H]⁺.

### Intermediate 131

### Methyl 6-chloro-7-(1-ethyl-5-((((5-(((8-((4-methoxybenzyl)oxy)quinolin-6-yl)thio)methyl)-1-methyl-1H-pyrazol-3-yl)methyl)thio)methyl)-3-methyl-1H-pyrazol-4-yl)-3-(3-hydroxypropyl)-1-methyl-1H-indole-2-carboxylate

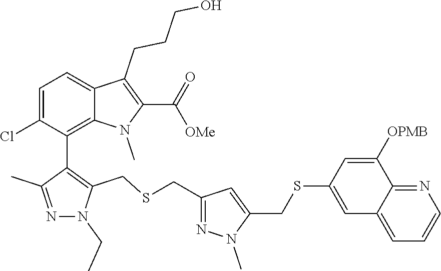

Intermediate 131 was synthesized from Intermediate 130 and Intermediate 11 following a procedure for the preparation of Intermediate 21. MS (LCMS) 839.7 [M+H]⁺.

### Intermediate 132

### Methyl 6-chloro-7-(1-ethyl-5-((((5-(((8-hydroxyquinolin-6-yl)thio)methyl)-1-methyl-1H-pyrazol-3-yl)methyl)thio)methyl)-3-methyl-1H-pyrazol-4-yl)-3-(3-hydroxypropyl)-1-methyl-1H-indole-2-carboxylate

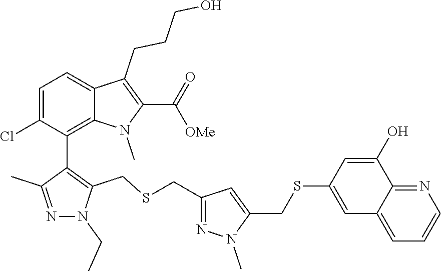 [SMILES: CCn1nc(C)c(-c2c(Cl)ccc3c(CCCO)c(C(=O)OC)n(C)c23)c1CSCc1cc(CSc2cc(O)c3ncccc3c2)n(C)n1]

Intermediate 132 was synthesized from Intermediate 131 following a procedure for the preparation of Intermediate 22. MS (LCMS) 719.6 [M+H]⁺.

### Intermediate 133

### Methyl (Z)-1⁶-chloro-2¹-ethyl-1¹,2³,6¹-trimethyl-1¹H,2¹H,6¹H-10-oxa-4,8-dithia-9(6,8)-quinolina-1(7,3)-indola-2(4,5),6(3 ,5)-dipyrazolacyclotridecaphane-1²-carboxylate

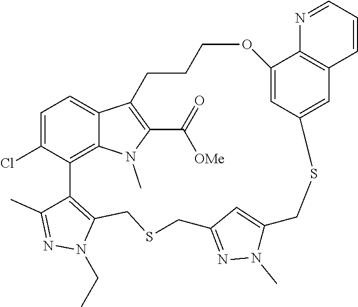 [SMILES: CCn1nc(C)c2c1CSCc1cc(n(C)n1)CSc1cc(c3ncccc3c1)OCCCc1c(C(=O)OC)n(C)c3c-2c(Cl)ccc13]

Intermediate 133 was synthesized from Intermediate 132 following a procedure for the preparation of Intermediate 23. MS (LCMS) 701.6 [M+H]⁺.

### Intermediate 134A

### (Rₐ)-(+)-Methyl (Z)-1⁶-chloro-2¹-ethyl-1¹,2³,6¹-trimethyl-9¹,9²,9³,9⁴-tetrahydro-1¹H,2¹H,6¹H-10-oxa-4,8-dithia-9(6,8)-quinolina-1(7,3)-indola-2(4,5),6(3,5)-dipyrazolacyclotridecaphane-1²-carboxylate

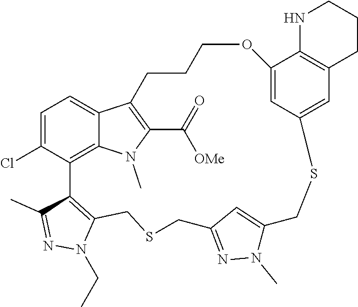

### Intermediate 134B

### (Sₐ)-(−)-Methyl (Z)-1⁶-chloro-2¹-ethyl-1¹,2³,6¹-trimethyl-9¹,9²,9³,9⁴-tetrahydro-1¹H,2¹H,6¹H-10-oxa-4, 8-dithia-9(6,8)-quinolina-1(7,3)-indola-2(4,5),6(3,5)-dipyrazolacyclotridecaphane-1²-carboxylate

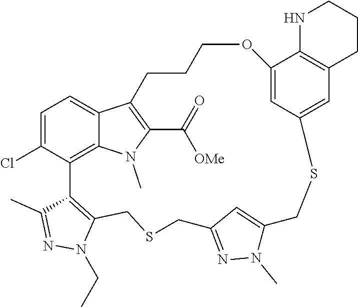

Intermediates 134A and 134B were synthesized from Intermediate 133 following a procedure for the preparation of Intermediates 24A and 24B to give racemic methyl (Z)-1⁶-chloro-2¹-ethyl-1¹,2³,6¹-trimethyl-9¹,9²,9³,9⁴-tetrahydro-1¹H,2¹H,6¹H-10-oxa-4,8-dithia-9(6,8)-quinolina-1(7,3)-indola-2(4,5),6(3,5)-dipyrazolacyclotridecaphane-1²-carboxylate (160 mg, 53%). The atropisomers were separated by chiral SFC chromatography (Chiralcel IC (30×250 mm) column, 40% MeOH) to give peak 1 (Intermediate 134A, 70 mg) and peak 2 (Intermediate 134B, 70 mg). Intermediate 134A: off-white solid; 99.5% chiral purity; MS (ESI) 705.2 [M+H]⁺. Intermediate 134B: off-white solid; 99.9% chiral purity; MS (ESI) 705.2 [M+H]⁺. The absolute stereochemistry of Intermediate 134A and Intermediate 134B was arbitrarily assigned.

### Example 14A

### (Rₐ)-(+)-(Z)-1⁶-Chloro-2¹-ethyl-1¹,2³,6¹-trimethyl-9¹,9²,9³,9⁴-tetrahydro-1¹H,2¹H,6¹H-10-oxa-4,8-dithia-9(6,8)-quinolina-1(7,3)-indola-2(4,5),6(3,5)-dipyrazolacyclotridecaphane carboxylic acid

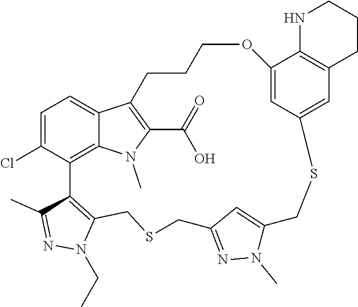

Example 14A was synthesized from Intermediate 134A following a procedure for the preparation of Example 1. Example 14A: (50 mg, 74%), off-white solid; 98.8% chiral purity; ¹H NMR (400 MHz, DMSO-d₆) δ 13.2 (br s, 1H), 7.72 (d, J=8.8 Hz, 1H), 7.11 (d, J=8.4 Hz, 1H), 6.59 (s, 1H), 6.00 (s, 1H), 5.33 (br s, 1H), 4.77 (s, 1H), 4.09-4.04 (m, 2H), 3.91-3.72 (m, 2H), 3.64-3.24 (m, 9H), 3.24-3.06 (m, 6H), 2.63 (s, 3H), 2.17-2.14 (m, 1H), 2.07-2.01 (m, 1H), 1.91 (s, 3H), 1.80-1.78 (m, 2H), 1.35 (t, J=7.2 Hz, 3H); MS (LCMS) 691.3 [M+H]⁺.

### Example 14B

### (Sₐ)-(−)-(Z)-1⁶-Chloro-2¹-ethyl-1¹,2³,6¹-trimethyl-9¹,9²,9³,9⁴-tetrahydro-1¹H,2¹H,6¹H-10-oxa-4,8-dithia-9(6,8)-quinolina-1(7,3)-indola-2(4,5),6(3,5)-dipyrazolacyclotridecaphane-1²-carboxylic acid

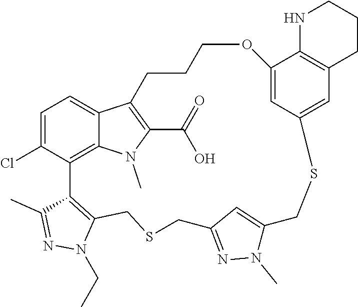

Example 14B was synthesized from Intermediate 134B following a procedure for the preparation of Example 1. Example 14B: (47 mg, 66%), off-white solid; 99.0% chiral purity; ¹H NMR (400 MHz, DMSO-d₆) δ 13.2 (br s, 1H), 7.71 (d, J=8.8 Hz, 1H), 7.11 (d, J=8.4 Hz, 1H), 6.59 (s, 1H), 5.99 (s, 1H), 5.33 (br s, 1H), 4.76 (s, 1H), 4.12-4.06 (m, 2H), 4.00-3.60 (m, 2H), 3.64-3.44 (m, 9H), 3.33-3.09 (m, 6H), 2.66-2.63 (m, 3H), 2.15-2.13 (m, 1H), 2.00-1.97 (m, 1H), 1.91 (s, 3H), 1.80-1.77 (m, 2H), 1.32 (t, J=7.2 Hz, 3H); MS (LCMS) 691.3 [M+H]+. The absolute stereochemistry of Example 14A and Example 14B was arbitrarily assigned.

### Example A

### Mcl-1 Homogeneous Time Resolved Fluorescence (HTRF) Assay

Binding to Bcl-2 proteins Mcl-1 was assessed using an HTRF assay. Background: FAM-Bak/Bad binds to surface pocket of the Bcl-2 protein family. This binding can be monitored by HTRF signals between anti-GST-Tb and FAM-peptide using GST-tagged Bcl proteins. Assay conditions: 4 nM Mcl-1, 100 nM FAM-Bak peptide, in 20 mM K Phosphate, pH 7.5, 50 mM NaCl, 1 mM EDTA, 0.005% Triton X-100 and 1% DMSO (final). Assay procedure: Compounds were tested in 10-dose IC₅₀ mode, in singlicate, with 3-fold serial dilution starting at 10 μM or 1 μM. Compound stock solutions were added to protein solution using Acoustic technology. The compounds were then incubated with protein for 10 min at rt. The respective FAM labeled peptide was added and incubated for another 10 min. Anti-GST-Tb was added. After 60 min at rt, the HTRF fluorescence signal ratio was measured. Curve fits were performed in GraphPad Prism 4 with “sigmoidal dose-response (variable slope)”; 4 parameters with Hill Slope. The results are shown in Table 1.

### Example B

### NCI-H929 Cell Proliferation Assay

Cell proliferation was measured using the CellTiter-Glo® Luminescent Cell Viability Assay. The assay involved the addition of a single reagent (CellTiter-Glo® Reagent) directly to cells cultured in serum-supplemented medium. NCI-H929 (ATCC CRL-9068) cells were cultured according to ATCC recommendations and were seeded at 3,000 cells per well.

Each compound evaluated was prepared as a DMSO stock solution (10 mM). Compounds were tested in duplicate on each plate, with a 10-point serial dilution curve (1:3 dilution). Compound treatment (1.0 μL) was added from the compound dilution plate to the cell plate. The highest compound concentration was 10 μM (final), with a 0.1% final DMSO concentration. Plates were then incubated at 37° C., 5% CO₂. After 72 h of compound treatment, cell plates were equilibrated at rt for approximately 30 mins. An equi-volume amount of CellTiter-Glo® Reagent (40 μL) was added to each well. Plates were mixed for 2 mins on an orbital shaker to induce cell lysis and then incubated at rt for 10 mins to stabilize the luminescent signal. Luminescence was recorded using an Envision plate reader according to CellTiter-Glo protocol. IC₅₀ of each compound was calculated using GraphPad Prism by nonlinear regression analysis. IC₅₀ values are provided in Table 1.

TABLE 1

| Examples | Mc1-1 IC₅₀ (nM) | H929 IC₅₀ (nM) |
|---|---|---|
| 1A | A | A |
| 1B | ND | C |
| 2A | A | A |
| 2B | ND | C |
| 3A | ND | C |
| 3B | ND | A |
| 4A | ND | C |
| 4B | ND | A |
| 5A | ND | A |
| 5B | ND | C |
| 6A | ND | A |
| 6B | ND | C |
| 7A | ND | B |
| 7B | ND | B |
| 8A | ND | C |
| 8B | ND | B |
| 9A | ND | C |
| 9B | ND | A |
| 10A | ND | C |
| 10B | ND | A |
| 11A | ND | ND |
| 11B | ND | ND |
| 12A | ND | C |
| 12B | ND | B |
| 13A | ND | C |
| 13B | ND | C |
| 14A | ND | A |
| 14B | ND | C |
| AMG176 | A | B |
| AZD5991 | A | A |
| S64315 | A | A |

Mc1-1 Binding Assay (IC₅₀): A = a single IC₅₀ ≤ 10 nM;
B = a single IC₅₀ > 10 nM and < 100 nM;
C = a single IC₅₀ ≥ 100 nM.
For H929 CTG IC₅₀: A = a single IC₅₀ ≤ 100 nM;
B = a single IC₅₀ > 100 nM and < 1000 nM;
C = a single IC₅₀ ≥ 1000 nM.

Furthermore, although the foregoing has been described in some detail by way of illustrations and examples for purposes of clarity and understanding, it will be understood by those of skill in the art that numerous and various modifications can be made without departing from the spirit of the present disclosure. Therefore, it should be clearly understood that the forms disclosed herein are illustrative only and are not intended to limit the scope of the present disclosure, but rather to also cover all modification and alternatives coming with the true scope and spirit of the invention.

1. A compound of Formula (I), or a pharmaceutically salt thereof, wherein the compound has the structure: 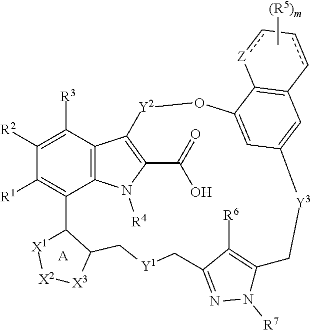
R¹, R², R³ and R⁶ are each independently hydrogen, halogen, an unsubstituted C₁₋₄ alkyl or an unsubstituted C₁₋₄ haloalkyl;
R⁴ and R⁷ are each independently hydrogen, an optionally substituted C₁₋₄ alkyl, an optionally substituted C₃₋₆ monocyclic cycloalkyl or an unsubstituted C₁₋₄ haloalkyl;
X¹, X² and X³ are each independently NR⁸ or CR⁹; and wherein Ring A is an aromatic ring;
R⁸ and R⁹ are each independently absent, hydrogen, halogen, cyano, an optionally substituted C₁₋₄ alkyl, an optionally substituted C₁₋₁ alkoxy, an optionally substituted C₃₋₆ monocyclic cycloalkyl, an optionally substituted C₃₋₆ bicyclic cycloalkyl, a mono-substituted amine or a di-substituted amine; or
the substituent attached to X¹ and the substituted attached to X² are taken together to form Ring B fused to Ring A; X³ is NR⁸ or CR⁹; and wherein Ring A and Ring B form an optionally substituted heteroaryl or an optionally substituted heterocyclyl; or
the substituent attached to X² and the substituted attached to X³ are taken together to form Ring C fused to Ring A; X¹ is NR⁸ or CR⁹; and wherein Ring A and Ring C form an optionally substituted heteroaryl or an optionally substituted heterocyclyl;
Y¹ is O, S, SO, SO₂, CH₂, CF₂ or NR^{10A};
Y² is an optionally substituted C₁₋₄ alkylene, and when Y² is substituted, each substituent is independently halogen or an unsubstituted C₁₋₄ alkyl;
Y³ is O, S, SO, SO₂, CH₂, CF₂ or NR^{10B};
R^{10A} and R^{10B} are independently hydrogen or an optionally substituted C₁₋₄ alkyl;
Z is NH or NCH₃;
each [symbol] is a single bond;
m is 0, 1 or 2; and
each R⁵ is independently halogen or an optionally substituted C₁₋₄ alkyl; and
provided that
when Y¹, Y² and Y³ are:
(1) Y¹ and Y³ are each S and Y² is —(CH₂)₃—;
(2) Y¹ is S, Y² is —(CH₂)₃— and Y³ is —(CH₂)—;
(3) Y¹ is NR^{10A}, Y² is —(CH₂)₃— and Y³ is S; or
(4) Y¹ is NR^{10A}, Y² is —(CH₂)₃— and Y³ is —(CH₂)—;
R¹ is chloro;
R², R³ and R⁶ are each hydrogen;
R⁴ and R⁷ are each methyl;
Z is NH and each [symbol] is a single bond; and m is 0;
then X¹, X² and X³ are not (1) X¹ is CR⁸, wherein R⁸ is an optionally substituted C₁₋₄ alkyl, X² is N and X³ is N(CH₃); and (2) X¹ is CR⁸, wherein R⁸ is an optionally substituted C₁₋₄ alkyl, X² is N(CH₃) and X³ is N.

2. (canceled)

3. The compound of claim 1, wherein R¹ is halogen.

4. (canceled)

5. The compound of claim 3, wherein the halogen is chloro.

6.-9. (canceled)

10. The compound of claim 1, wherein R², R³ and R⁶ are each hydrogen.

11.-26. (canceled)

27. The compound of claim 1, wherein R⁴ is an unsubstituted C₁₋₄ alkyl.

28.-37. (canceled)

38. The compound of claim 1, wherein R⁷ is an unsubstituted C₁₋₄ alkyl or an unsubstituted C₃₋₆ monocyclic cycloalkyl.

39. (canceled)

40. (canceled)

41. (canceled)

42. (canceled)

43. The compound of claim 1, wherein

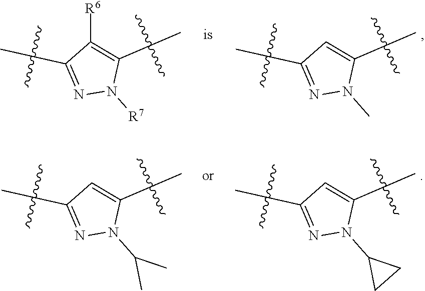

44. (canceled)

45. The compound of claim 1, wherein X¹, X² and X³ are each independently NR⁸ or CR⁹; Ring A is an aromatic ring; and R⁸ and R⁹ are each independently absent, hydrogen, halogen, cyano, an optionally substituted C₁₋₄ alkyl, an optionally substituted C₁₋₄ alkoxy, an optionally substituted C₃₋₆ monocyclic cycloalkyl, an optionally substituted C₃₋₆ bicyclic cycloalkyl, a mono-substituted amine or a di-substituted amine.

46. (canceled)

47. (canceled)

48. (canceled)

49. (canceled)

50. The compound of claim 1, wherein Ring A is

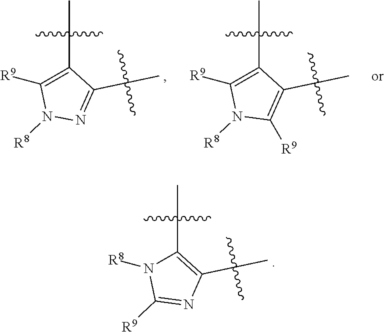

51. The compound of claim 1, wherein Ring A is

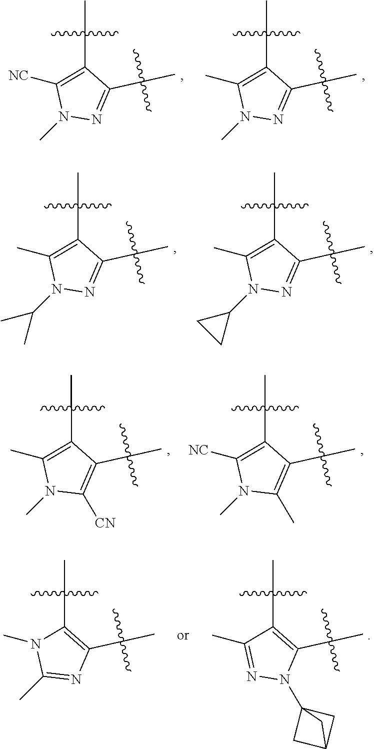

52. The compound of claim 1, wherein X¹ and X² are each independently NR⁸ or CR⁹; the substituent attached to X¹ and the substituted attached to X² are taken together to form Ring B fused to Ring A; X³ is NR⁸ or CR⁹; Ring A and Ring B form an optionally substituted heteroaryl or an optionally substituted heterocyclyl; and R⁸ and R⁹ are each independently absent, hydrogen, halogen, cyano, an optionally substituted C₁₋₄ alkyl, an optionally substituted C₁₋₄ alkoxy, an optionally substituted C₃₋₆ monocyclic cycloalkyl, an optionally substituted C₃₋₆ bicyclic cycloalkyl, a mono-substituted amine or a di-substituted amine.

53. (canceled)

54. (canceled)

55. (canceled)

56. (canceled)

57. (canceled)

58. (canceled)

59. The compound of claim 1, wherein Ring A fused to Ring B is selected from the group consisting of:

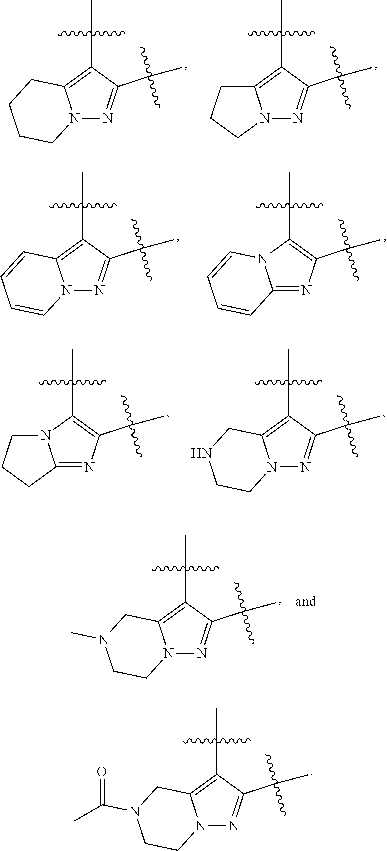

60.-67. (canceled)

68. The compound of claim 1, wherein m is 0.

69.-75. (canceled)

76. The compound of claim 1, wherein

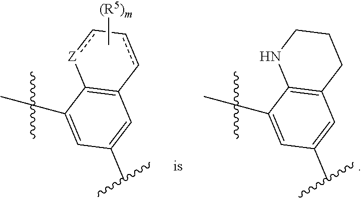

77. (canceled)

78. The compound of claim 1, wherein Y¹ is S or NR^{10A}, wherein R^{10A} is hydrogen or an optionally substituted C₁₋₄ alkyl.

79.-84. (canceled)

85. The compound of claim 1, wherein Y² is an unsubstituted C₁₋₄ alkylene.

86. (canceled)

87. The compound of claim 1, wherein Y² is —CH₂—, —CH₂CH₂—, —CH₂CH₂CH₂—, —CH₂CH₂CH₂CH₂—, —CH(CH₃)CH₂CH₂—, —CHFCH₂CH₂— or —CH₂CF₂CH₂—.

88. (canceled)

89. The compound of claim 1, wherein Y³ is S or CH₂.

90.-96. (canceled)

97. The compound of claim 1, wherein Z is NH.

98. (canceled)

99. The compound of claim 1, wherein the compound is selected from the group consisting of: 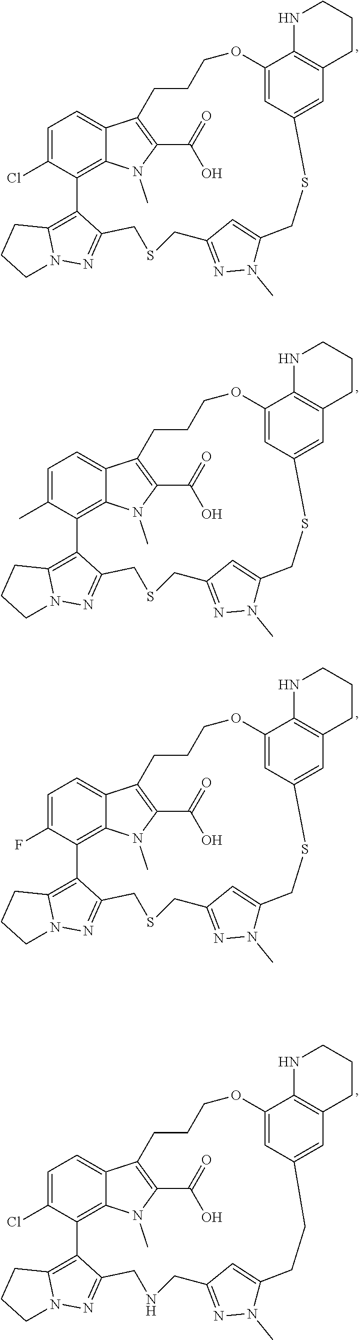 [SMILES: Cn1nc2cc1CSc1cc3c(c(c1)OCCCc1c(C(=O)O)n(C)c4c(c(Cl)ccc14)-c1c(nn4c1CCC4)CSC2)NCCC3 ; Cc1ccc2c3c(C(=O)O)n(C)c2c1-c1c(nn2c1CCC2)CSCc1cc(n(C)n1)CSc1cc2c(c(c1)OCCC3)NCCC2 ; Cn1nc2cc1CSc1cc3c(c(c1)OCCCc1c(C(=O)O)n(C)c4c(c(F)ccc14)-c1c(nn4c1CCC4)CSC2)NCCC3 ; Cn1nc2cc1CCc1cc3c(c(c1)OCCCc1c(C(=O)O)n(C)c4c(c(Cl)ccc14)-c1c(nn4c1CCC4)CNC2)NCCC3] 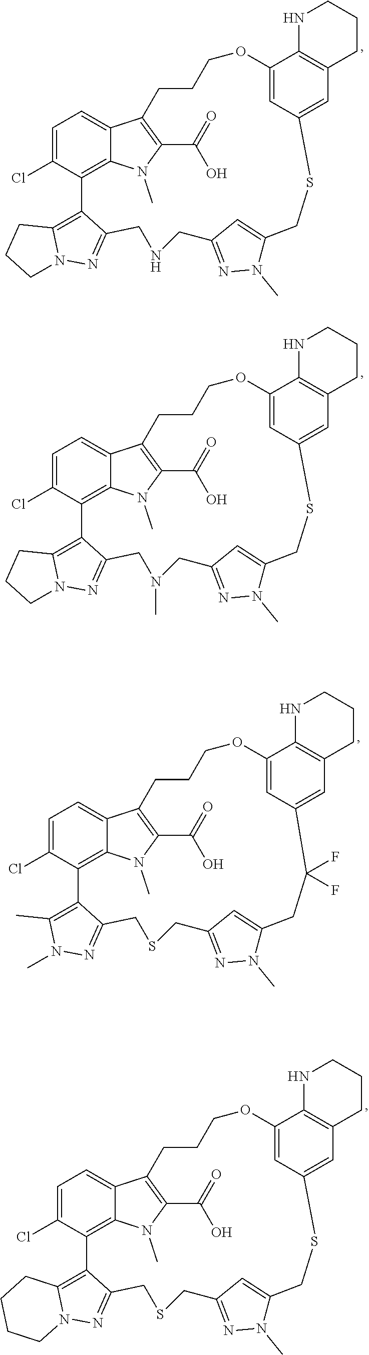 [SMILES: Cn1nc2cc1CSc1cc3c(c(c1)OCCCc1c(C(=O)O)n(C)c4c(c(Cl)ccc14)-c1c(nn4c1CCC4)CNC2)NCCC3 ; CN1Cc2cc(n(C)n2)CSc2cc3c(c(c2)OCCCc2c(C(=O)O)n(C)c4c(c(Cl)ccc24)-c2c(nn4c2CCC4)C1)NCCC3 ; Cc1c2c(nn1C)CSCc1cc(n(C)n1)CC(F)(F)c1cc3c(c(c1)OCCCc1c(C(=O)O)n(C)c4c-2c(Cl)ccc14)NCCC3 ; Cn1nc2cc1CSc1cc3c(c(c1)OCCCc1c(C(=O)O)n(C)c4c(c(Cl)ccc14)-c1c(nn4c1CCCC4)CSC2)NCCC3] 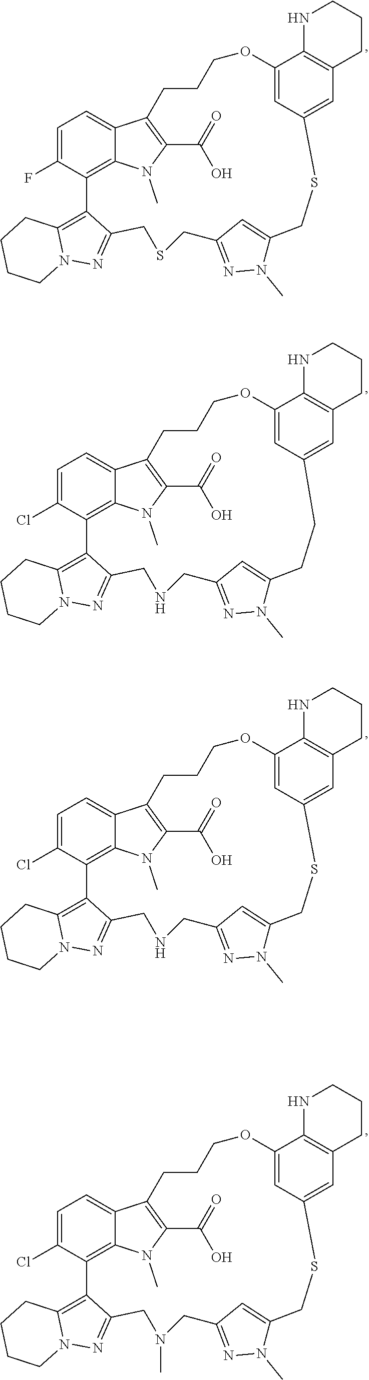 [SMILES: Cn1nc2cc1CSc1cc3c(c(c1)OCCCc1c(C(=O)O)n(C)c4c(c(F)ccc14)-c1c(nn4c1CCCC4)CSC2)NCCC3 ; Cn1nc2cc1CCc1cc3c(c(c1)OCCCc1c(C(=O)O)n(C)c4c(c(Cl)ccc14)-c1c(nn4c1CCCC4)CNC2)NCCC3 ; Cn1nc2cc1CSc1cc3c(c(c1)OCCCc1c(C(=O)O)n(C)c4c(c(Cl)ccc14)-c1c(nn4c1CCCC4)CNC2)NCCC3 ; CN1Cc2cc(n(C)n2)CSc2cc3c(c(c2)OCCCc2c(C(=O)O)n(C)c4c(c(Cl)ccc24)-c2c(nn4c2CCCC4)C1)NCCC3] 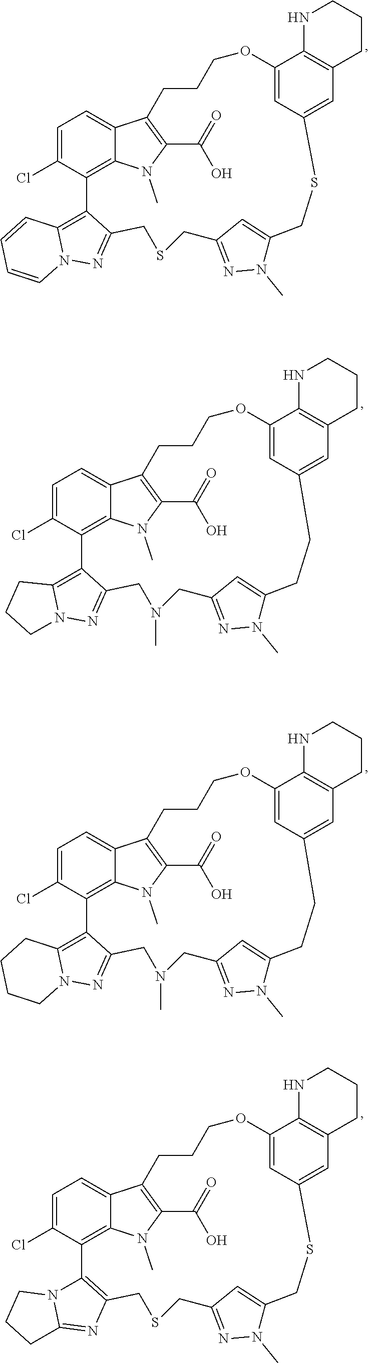 [SMILES: Cn1nc2cc1CSc1cc3c(c(c1)OCCCc1c(C(=O)O)n(C)c4c(c(Cl)ccc14)-c1c(nn4ccccc14)CSC2)NCCC3 ; CN1Cc2cc(n(C)n2)CCc2cc3c(c(c2)OCCCc2c(C(=O)O)n(C)c4c(c(Cl)ccc24)-c2c(nn4c2CCC4)C1)NCCC3 ; CN1Cc2cc(n(C)n2)CCc2cc3c(c(c2)OCCCc2c(C(=O)O)n(C)c4c(c(Cl)ccc24)-c2c(nn4c2CCCC4)C1)NCCC3 ; Cn1nc2cc1CSc1cc3c(c(c1)OCCCc1c(C(=O)O)n(C)c4c(c(Cl)ccc14)-c1c(nc4n1CCC4)CSC2)NCCC3] 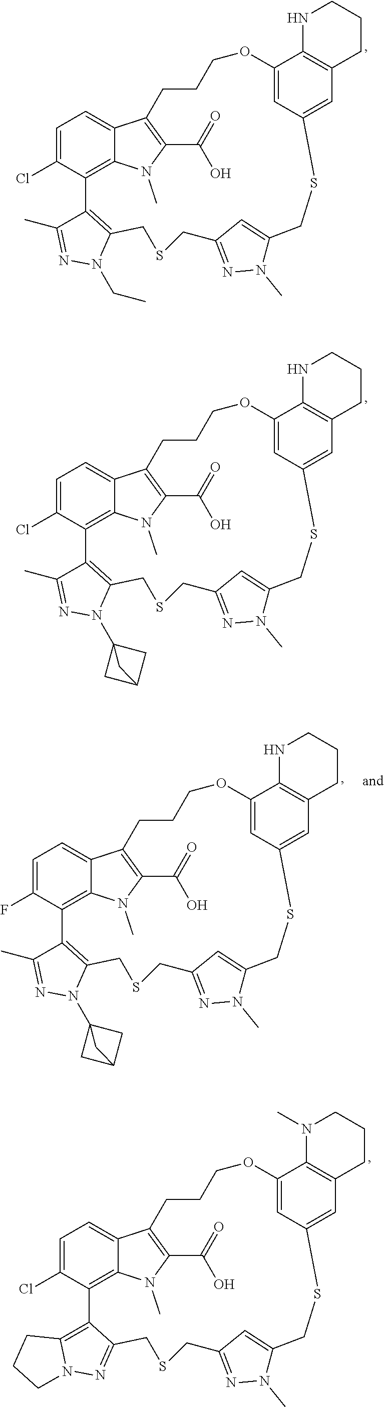 [SMILES: CCn1nc(C)c2c1CSCc1cc(n(C)n1)CSc1cc3c(c(c1)OCCCc1c(C(=O)O)n(C)c4c-2c(Cl)ccc14)NCCC3 ; Cc1nn(C23CC(C2)C3)c2c1-c1c(Cl)ccc3c(c(C(=O)O)n(C)c13)CCCOc1cc(cc3c1NCCC3)SCc1cc(nn1C)CSC2 ; Cc1nn(C23CC(C2)C3)c2c1-c1c(F)ccc3c(c(C(=O)O)n(C)c13)CCCOc1cc(cc3c1NCCC3)SCc1cc(nn1C)CSC2 ; CN1CCCc2cc3cc(c21)OCCCc1c(C(=O)O)n(C)c2c(c(Cl)ccc12)-c1c(nn2c1CCC2)CSCc1cc(n(C)n1)CS3]
or a pharmaceutically acceptable salt of any of the foregoing.

100. The compound of claim 99, wherein the compound is selected from the group consisting of: 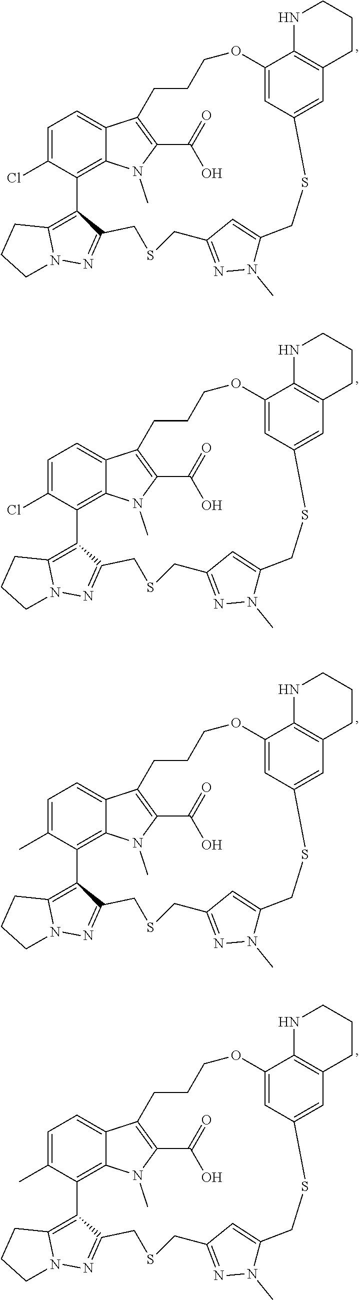 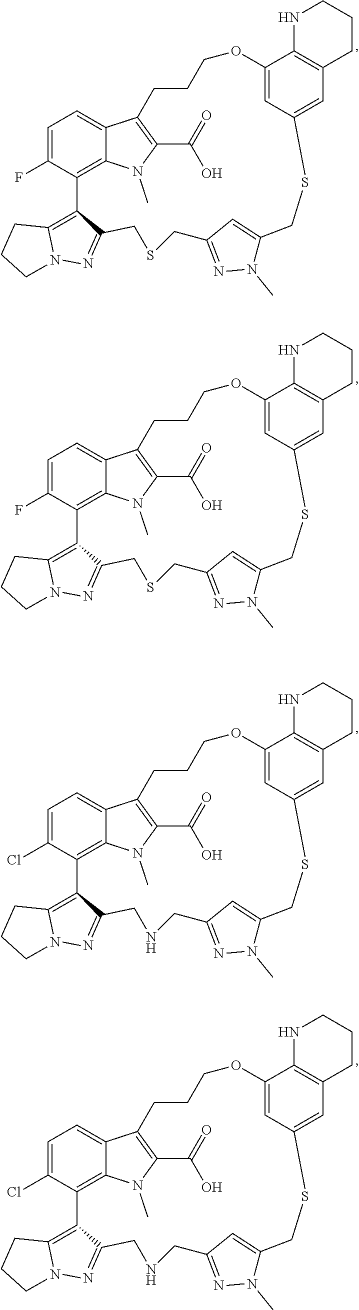 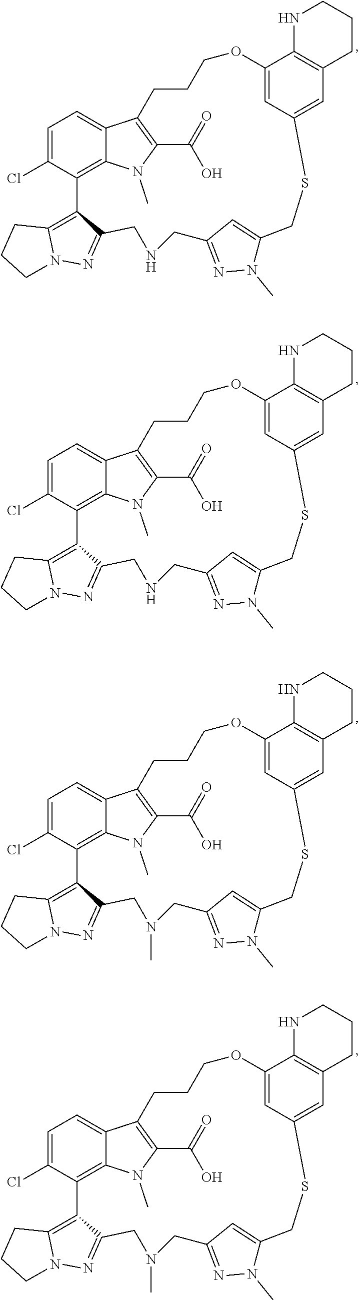 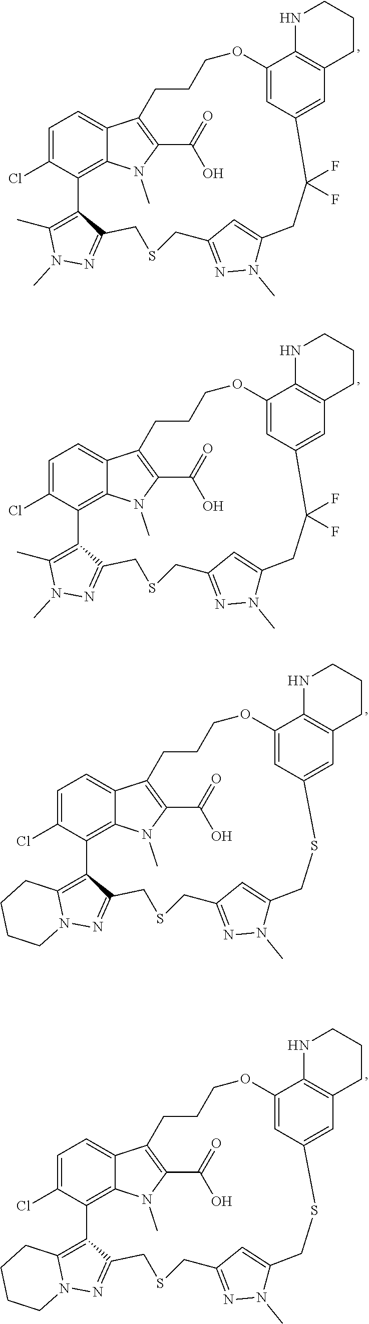 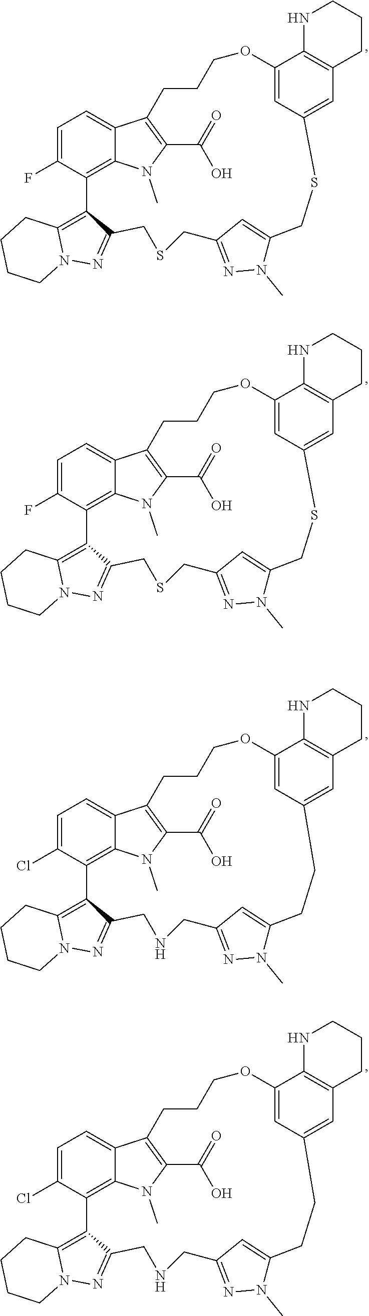 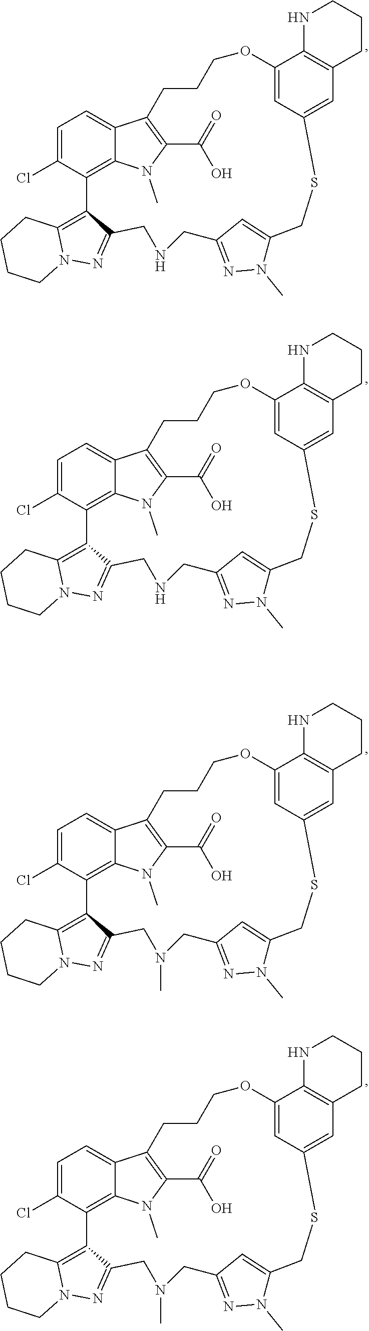 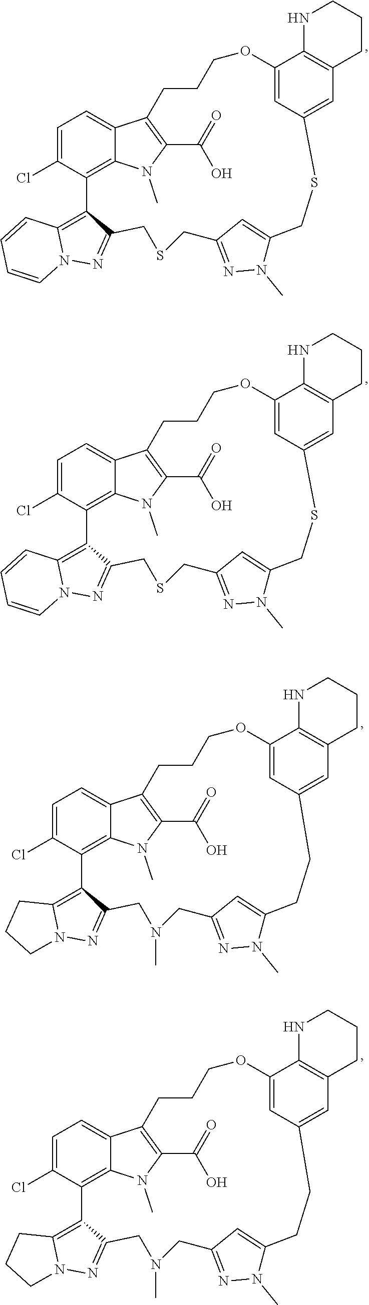 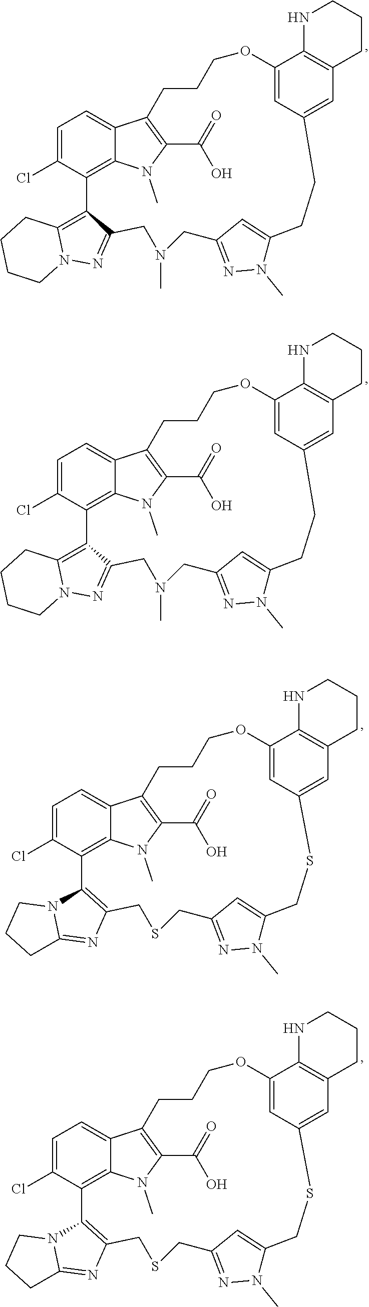 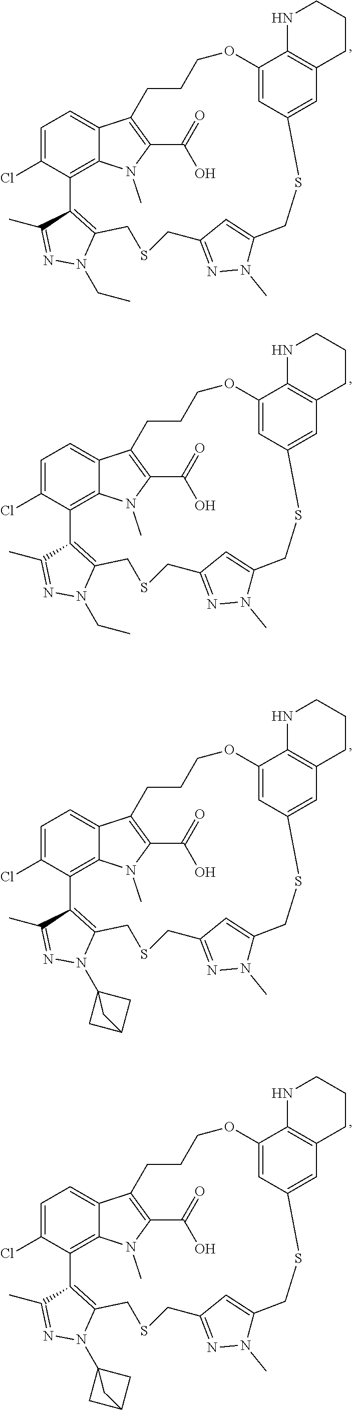 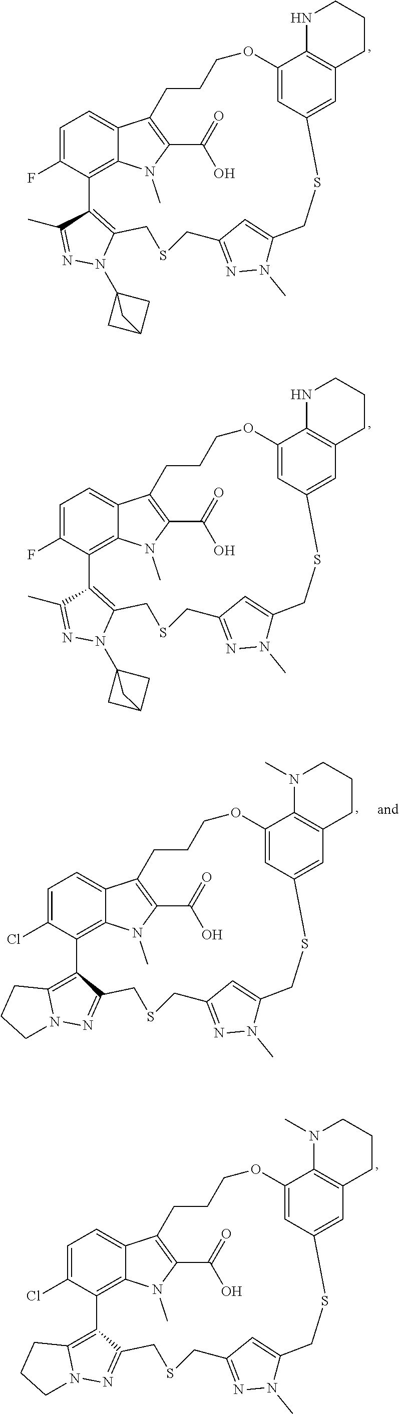
or a pharmaceutically acceptable salt of any of the foregoing.

101. A pharmaceutical composition comprising an effective amount of the compound of claim 1, or a pharmaceutically acceptable salt thereof, and a pharmaceutically acceptable carrier, diluent, excipient or combination thereof.

102. A method of ameliorating or treating a cancer comprising administering an effective amount of the compound of claim 1, or a pharmaceutically acceptable salt thereof, to a subject having the cancer, wherein the cancer is selected from the group consisting of a brain cancer, a cervicocerebral cancer, an esophageal cancer, a thyroid cancer, a small cell cancer, a non-small cell cancer, a breast cancer, a lung cancer , a stomach cancer, a gallbladder/bile duct cancer, a liver cancer, a pancreatic cancer, a colon cancer, a rectal cancer, an ovarian cancer, a choriocarcinoma, an uterus body cancer, an uterocervical cancer, a renal pelvis/ureter cancer, a bladder cancer, a prostate cancer, a penis cancer, a testicular cancer, a fetal cancer, Wilms' cancer, a skin cancer, malignant melanoma, a neuroblastoma, an osteosarcoma, an Ewing's tumor, a soft part sarcoma, an acute leukemia, a chronic lymphatic leukemia, a chronic myelocytic leukemia, polycythemia vera, a malignant lymphoma, multiple myeloma, a Hodgkin's lymphoma, and a non-Hodgkin's lymphoma.

103. A method for inhibiting replication of a malignant growth or a tumor comprising contacting the malignant growth or the tumor with an effective amount of the compound of claim 1, or a pharmaceutically acceptable salt thereof, wherein the malignant growth or tumor is due to a cancer selected from the group consisting of a brain cancer, a cervicocerebral cancer, an esophageal cancer, a thyroid cancer, a small cell cancer, a non-small cell cancer, a breast cancer, a lung cancer , a stomach cancer, a gallbladder/bile duct cancer, a liver cancer, a pancreatic cancer, a colon cancer, a rectal cancer, an ovarian cancer, a choriocarcinoma, an uterus body cancer, an uterocervical cancer, a renal pelvis/ureter cancer, a bladder cancer, a prostate cancer, a penis cancer, a testicular cancer, a fetal cancer, Wilms' cancer, a skin cancer, malignant melanoma, a neuroblastoma, an osteosarcoma, an Ewing's tumor, a soft part sarcoma, an acute leukemia, a chronic lymphatic leukemia, a chronic myelocytic leukemia, polycythemia vera, a malignant lymphoma, multiple myeloma, a Hodgkin's lymphoma, and a non-Hodgkin's lymphoma.

104. A method for ameliorating or treating a malignant growth or tumor comprising administering an effective amount of the compound of claim 1, or a pharmaceutically acceptable salt thereof, to a subject in need thereof, wherein the malignant growth or tumor is due to a cancer selected from the group consisting of a brain cancer, a cervicocerebral cancer, an esophageal cancer, a thyroid cancer, a small cell cancer, a non-small cell cancer, a breast cancer, a lung cancer , a stomach cancer, a gallbladder/bile duct cancer, a liver cancer, a pancreatic cancer, a colon cancer, a rectal cancer, an ovarian cancer, a choriocarcinoma, an uterus body cancer, an uterocervical cancer, a renal pelvis/ureter cancer, a bladder cancer, a prostate cancer, a penis cancer, a testicular cancer, a fetal cancer, Wilms' cancer, a skin cancer, malignant melanoma, a neuroblastoma, an osteosarcoma, an Ewing's tumor, a soft part sarcoma, an acute leukemia, a chronic lymphatic leukemia, a chronic myelocytic leukemia, polycythemia vera, a malignant lymphoma, multiple myeloma, a Hodgkin's lymphoma, and a non-Hodgkin's lymphoma.

105. A method for inhibiting the activity of Mcl-1 in a cell comprising contacting the cell with an effective amount of the compound of claim 1, or a pharmaceutically acceptable salt thereof, wherein the cell is a cancer cell and the cancer is selected from the group consisting of a brain cancer, a cervicocerebral cancer, an esophageal cancer, a thyroid cancer, a small cell cancer, a non-small cell cancer, a breast cancer, a lung cancer , a stomach cancer, a gallbladder/bile duct cancer, a liver cancer, a pancreatic cancer, a colon cancer, a rectal cancer, an ovarian cancer, a choriocarcinoma, an uterus body cancer, an uterocervical cancer, a renal pelvis/ureter cancer, a bladder cancer, a prostate cancer, a penis cancer, a testicular cancer, a fetal cancer, Wilms' cancer, a skin cancer, malignant melanoma, a neuroblastoma, an osteosarcoma, an Ewing's tumor, a soft part sarcoma, an acute leukemia, a chronic lymphatic leukemia, a chronic myelocytic leukemia, polycythemia vera, a malignant lymphoma, multiple myeloma, a Hodgkin's lymphoma, and a non-Hodgkin's lymphoma.
